(19) United States
(12) Patent Application Publication
(10) Pub. No.: US 2024/0393336 A1
(43) Pub. Date: Nov. 28, 2024

(54) BIOMARKERS FOR COLORECTAL CANCER

(71) Applicant: Vision Tech Bio Pty Ltd, Parkville, Victoria (AU)

(72) Inventors: Trevor LOCKETT, Denistone, New South Wales (AU); Eduardo VOM, Parkville, Victoria (AU); Craig LEWIS, Parkville, Victoria (AU); Michael BUCKLEY, Pennant Hills, New South Wales (AU); Louise MILLER, Parkville, Victoria (AU); Christian DAISH, Parkville, Victoria (AU)

(73) Assignee: Vision Tech Bio Pty Ltd, Parkville, Victoria (AU)

(21) Appl. No.: 18/287,621

(22) PCT Filed: Apr. 20, 2022
(86) PCT No.: PCT/AU2022/050362

§ 371 Date: Oct. 19, 2023

(30) Foreign Application Priority Data
Apr. 20, 2021 (AU) 2021901164

(51) Int. Cl. G01N 33/574

(52) U.S. Cl. CPC G01N 33/57419; G01N 2333/475; G01N 2333/91215

(57) ABSTRACT

The present invention relates to the identification of biomarkers which are associated with a higher risk of advanced colorectal Neoplasia (ACN), for example colorectal cancer (CRC), in a subject. The detection and measurement of these biomarkers in a biological sample may be used to inform the clinician as to whether further invasive procedures including colonoscopy or sigmoidoscopy are required to provide a definitive diagnosis of colorectal cancer in the subject.

### CROSS-REFERENCE TO RELATED APPLICATIONS

This application is the U.S. National Stage Application, pursuant to 35 U.S.C. § 371 of PCT International Application No. PCT/AU2022/050362, filed 20 Apr. 2022, designating the United States and published in English, which claims priority to and the benefit of Australian Patent Application No. 2021901164, filed 20 Apr. 2021, the entire contents of each of which are incorporated by reference herein.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

### REFERENCE TO SEQUENCE LISTING

The entire content of the electronic submission of the sequence listing is incorporated by reference in its entirety for all purposes.

### FIELD

The present invention relates to the identification of biomarkers which are associated with a higher risk of advanced colorectal Neoplasia (ACN), for example colorectal cancer (CRC), in a subject. The detection and measurement of these biomarkers in a biological sample may be used to inform the clinician as to whether further invasive procedures including colonoscopy or sigmoidoscopy are required to provide a definitive diagnosis of colorectal cancer in the subject.

### BACKGROUND

Colorectal cancer (CRC), also referred to as colon cancer or bowel cancer, is the third most common cause of cancer in men and the second most common cause of cancer in women worldwide. In 2018, there were over 1.8 million new cases of CRC with Australia ranking eleventh highest in the world with an age-standardised rate of around 37 per 100,000. Unfortunately, 30-50% of patients have occult or overt metastases at presentation and once tumours have metastasized prognosis is very poor with a five year survival of less than 10% (Etzioni et al., (2003) Nat Rev Cancer 3:243-252). By contrast, greater than 90% of patients who present while the tumour is still localised will still be alive after 5 years and can be considered cured. The early detection of colorectal lesions would therefore significantly reduce the impact of colon cancer.

The current screening assays in widespread use for the diagnosis of colorectal cancer are the faecal occult blood test (FOBT), flexible sigmoidoscopy, and colonoscopy (Lieberman, (2010) Gastroenterology 138:2115-2126). While the specificity of FOBT for colorectal cancer is quite high (92-95%), the proportion of FOBT positive subjects found to have colorectal cancer at colonoscopy is low (˜3-4%). All positive FOBT must therefore be followed up with colonoscopy. Sampling is done by individuals at home and requires at least two consecutive faecal samples to be analysed to achieve optimal sensitivity. Some versions of the FOBT also require dietary restrictions prior to sampling. FOBT also lacks sensitivity for early stage cancerous lesions as these do not bleed into the bowel as frequently as more advanced cancers yet it is these early lesions for which treatment is most successful.

While FOBT screening does result in reduction of mortality due to colorectal cancer it suffers from a low compliance rate (30-40%), due in part to the unpalatable nature of the test, which limits its usefulness as a screening tool. Colonoscopy is the current gold standard and has a specificity of greater than 90% but it is intrusive and costly with a small but finite risk of complications (2.1 per 1000 procedures) (Levin, (2004) Gastroenterology 127:1841-1844). Development of a rapid, specific, cheap blood based assay would overcome compliance issues commonly seen with other screening tests and capture more subjects at risk of CRC.

### SUMMARY

The present disclosure is based on the identification of blood based biomarkers associated with a higher risk of colorectal cancer in a subject. The inventors have also identified biomarker combinations which are gender specific for males and females. The invention relates to specific combinations of biomarkers as well as the methods for diagnosing and detecting colorectal cancer and to methods for the identification of a subject a risk of colorectal cancer.

Accordingly, in a first aspect, the present disclosure provides a method for diagnosing colorectal cancer and/or identifying a subject suspected of having, or at a greater risk of having colorectal cancer, the method comprising:
determining a measurement for a panel of biomarkers in a biological sample obtained from the subject, the panel comprising at least brain derived neurotropic factor (BDNF) and tumour M2-PK;
wherein the measurement comprises measuring a level of each of the biomarkers in the panel.

In one example according to the first aspect, determining a measurement comprises detecting at least BDNF and M2PK (and one or more other biomarkers) in the biological sample by contacting the sample with detectable binding agents that specifically bind to the biomarkers. In a further example, the method comprises detecting specific binding between the specific binding agents and the biomarkers using a detection assay. In one example, the detection assay in an ELISA assay. In a further example, determining a measurement comprises measuring the concentration of biomarker in the biological sample. In a further example, determining a measurement comprises performing a statistical analysis. In another example, the method comprises imputing the biomarker concentrations into an algorithm as described herein.

In one example according to any aspect herein, the method further comprises determining a measurement of one or more additional biomarkers selected from the group consisting of DKK3, TGFβ1, IGFBP2, TIMP1, IL6, IL8, TNFα, IGFII, Lipocalin, M30, M65, Mac2BP, MMP1, MMP7, MIP1B and IL13. In another example, the one or more additional biomarkers are selected from the group consisting of DKK3, TGFβ1, IGFBP2, TNFα, TIMP1, IL8, MIP1B and Mac2BP.

In one example according to the first aspect, the method comprises determining a measurement for a panel of at least three biomarkers, wherein the panel comprises at least BDNF and M2PK.

In a further example, the at least three biomarkers comprise BDNF and M2PK and a further biomarker selected from the group consisting of DKK-3, TNFα, IL-8, MAC2BP and IGFBP2.

In one example according to the first aspect, the three biomarker panels comprise or consist of:
i) BDNF, M2PK, DKK-3; ii) BDNF, M2PK, TNFα; iii) BDNF, M2PK, IL-8; iv) BDNF, M2PK, MAC2BP; or v) BDNF, M2PK, IGFBP2.

In another example according to the first aspect, the method comprises determining a measurement for a panel of at least four biomarkers, wherein the panel comprises at least BDNF and M2PK. In a further example, the at least four biomarkers comprise BDNF, M2PK and two biomarkers selected from the group consisting of DKK3, TNFα, IGFBP2, TIMP1, MIP1β, MMP7, MMP1, IGFII and IL-8.

In a further example, the at least four biomarkers comprise BDNF, M2PK and two biomarkers selected from the group consisting of DKK3, IGFBP2, TIMP1, and IL-8. In a further example, the at least four biomarkers comprise BDNF, M2PK and two biomarkers selected from the group consisting of DKK3, IGFBP2 and TIMP1. In one example according to the first aspect, the four biomarkers comprise DKK3, M2PK, IGFPB2 and BDNF.

In another example according to the first aspect, the four biomarkers comprise or consist of:
i) BDNF, M2PK, IL8, TNFα; ii) BDNF, M2PK, IL8, MIP1B; iii) BDNF, M2PK, IL8, DKK3; iv) BDNF, M2PK, MMP1, DKK3; v) BDNF, M2PK, IL8, IGFBP2; vi) BDNF, M2PK, IL8, TIMP1; vii) BDNF, M2PK, MMP7, TNFα; viii) BDNF, M2PK, IL8, MMP1; ix) BDNF, M2PK, IGFBP2, TNFα; or x) BDNF, M2PK, IL8, IGFII.

In another example according to the first aspect, the method comprises determining a measurement for a panel of at least four biomarkers, wherein the panel comprises at least BDNF, M2PK, IL-8 and a further biomarker selected from the group consisting of DKK3, TNFα, IGFBP2, TIMP1, MIP1β, MMP7, MMP1 and IGFII.

In another example according to the first aspect, the method comprises determining a measurement for a panel of at least five biomarkers, wherein the panel comprises at least BDNF and M2PK and three or more biomarkers selected from the group consisting of DKK3, TNFα, TGFBETA1, LIPOCALIN, IGFBP2, MAC2BP, MIP1β, MMP7, and IL-8.

In one example, the five biomarker panels comprise or consists of:
i) BDNF; M2PK; IL8; TNFA; MIP1β; ii) BDNF; M2PK; IL8; LIPOCALIN; DKK3; iii) BDNF; M2PK; IL8; TGFβ1; DKK3; iv) BDNF; M2PK; IL8; TNFA; TGFβ1; v) BDNF; M2PK; IGFBP2; TNFA; DKK3; vi) BDNF; M2PK; IL8; MMP7; DKK3; vii) BDNF; M2PK; IL8; TNFA; DKK3; viii) BDNF; M2PK; IL8; IGFBP2; MIP1β; ix) BDNF; M2PK; IL8; MAC2BP; DKK3; or x) BDNF; M2PK; IL8; TNFA; MAC2BP.

In another example according to the first aspect, the method comprises determining a measurement for a panel of at least five biomarkers, wherein the panel comprises at least BDNF and M2PK and three or more biomarkers selected from the group consisting of TIMP1, DKK3, TNFα, TGFBETA1, LIPOCALIN, IGFBP2, MAC2BP, MIP1β, MMP7, and IL-8. In one example, the panel comprises at least BDNF and M2PK and three or more biomarkers selected from the group consisting of TIMP1, DKK3, IGFBP2, MAC2BP, IL13 and IL-8.

In one example, the five biomarker panel comprises PKM2 (also referred to as M2PK), BDNF, DKK3, IGFBP2 and TIMP1.

In one example, the five biomarker panel comprises DKK3, M2PK, Mac2BP, IGFBP2 and BDNF.

In another example according to the first aspect, the method comprises determining a measurement for a panel of at least six biomarkers, wherein the panel comprises at least BDNF and M2PK and four or more biomarkers selected from the group consisting of DKK3, TNFα, IGFBP2, MIP1β, TGFβ1, MMP1, MAC2BP, IGFII, LIPOCALIN, IL6, M30 and IL-8.

In one example according to the first aspect, the six biomarker panels comprise or consist of:
i) BDNF; M2PK; MIP1β; DKK3; IGFBP2; TNFα; ii) BDNF; M2PK; MIP1β; DKK3; IGFBP2; MMP1; iii) BDNF; M2PK; MAC2BP; DKK3; IGFBP2; TNFα; iv) BDNF; M2PK; LIPOCALIN; DKK3; IGFBP2; TNFα; v) BDNF; M2PK; IL6; DKK3; IGFBP2; TNFα; vi) BDNF; M2PK; IL8; DKK3; LIPOCALIN; TGFβ1; vii) BDNF; M2PK; IL8; MIP1B; TGFβ1; TNFα; viii) BDNF; M2PK; IL8; MIP1B; IGFII; TNFα; ix) BDNF; M2PK; IL8; M30; TGFβ1; TNFα; or x) BDNF; M2PK; IL8; DKK3; IGFII; TNFα.

In another example according to the first aspect, the method comprises determining a measurement for a panel of at least seven biomarkers, wherein the panel comprises at least BDNF and M2PK and five or more biomarkers selected from the group consisting of TNFα, DKK3, MIP1β, IL8, MMP1, IGFBP2, LIPOCALIN, MAC2BP, IL6, MMP7, IGFII, M65, TIMP1 and TGFβ1. In one example, the method comprises determining a measurement for a panel of at least seven biomarkers, wherein the panel comprises at least BDNF, M2PK and TNFα and four or more biomarkers selected from the group consisting of DKK3, MIP1β, IL8, MMP1, IGFBP2, LIPOCALIN, MAC2BP, IL6, MMP7, IGFII, M65, TIMP1 and TGFβ1. In one example, the method comprises determining a measurement for a panel of at least seven biomarkers, wherein the panel comprises at least BDNF, M2PK, TNFα and DKK3 and three or more biomarkers selected from the group consisting of MIP1β, IL8, MMP1, IGFBP2, LIPOCALIN, MAC2BP, IL6, MMP7, IGFII, M65, TIMP1 and TGFβ1.

In another example according to the first aspect, the method comprises determining a measurement for a panel of at least eight biomarkers, wherein the panel comprises at least BDNF and M2PK and six or more biomarkers selected from the group consisting of DKK3, TNFα, MIP1β, IL8, MMP1, IGFBP2, LIPOCALIN, MAC2BP, IL6, MMP7, IGFII, M65, TIMP1, TGFβ1 and IL13. In one example, the panel comprises BDNF, M2PK and DKK3 and five or more biomarkers selected from the group consisting of TNFα, MIP1β, IL8, MMP1, IGFBP2, LIPOCALIN, MAC2BP, IL6, MMP7, IGFII, M65, TIMP1, TGFβ1 and IL13. In one example, the panel comprises BDNF, M2PK, DKK3 and IGFBP2 and four or more biomarkers selected from the group consisting of TNFα, MIP1β, IL8, MMP1, LIPOCALIN, MAC2BP, IL6, MMP7, IGFII, M65, TIMP1, TGFβ1 and IL13.

In another example according to the first aspect, the method comprises determining a measurement for a panel of at least four biomarkers, wherein the panel comprises at least BDNF, DKK3 and M2PK and one or more biomarkers selected from the group consisting of TNFα, MIP1β, IL8, MMP1, IGFBP2, LIPOCALIN, MAC2BP, IL6, MMP7, IGFII, M65, TIMP1, TGFβ1 and IL13.

In another example according to the first aspect, the method comprises determining a measurement for a panel of at least five biomarkers, wherein the panel comprises at least DKK3, IGFBP2, BDNF and M2PK and one or more biomarkers selected from the group consisting of TNFα, MIP1β, IL8, MMP1, LIPOCALIN, MAC2BP, IL6, MMP7, IGFII, M65, TIMP1, TGFβ1 and IL13.

In another example according to the first aspect, the method comprises determining a measurement for a panel of biomarkers present in Table 7, 8, 9, 10, 11, 12, 13, 14 or 15.

In some examples, the methods of the disclosure also contemplate the inclusion of the subject's age and/or gender as an biomarker in a biomarker panel described herein.

In some examples, the methods of the disclosure also contemplate the inclusion of the subject's age as an biomarker in a biomarker panel described herein. In one example, the subject's age is their age in years. In another example according to the first aspect, the method comprises determining a measurement for a panel of biomarkers present in Table 16, 17, 18, 19, 20, 21, 22, 23 or 24.

In some examples, the methods of the disclosure also contemplate the inclusion of the subject's gender as an biomarker in a biomarker panel described herein. Gender can be factored into the method by either separating the samples from males and females and analysing them separately. Alternatively, gender can be factored into the logistic regression algorithm by assigning an arbitrary value for females and a different arbitrary value for males. In one example, the subject's gender can be factored into the logistic regression algorithm by assigning an arbitrary value for males and females (for example, 1.1 for females and 1.0 for males).

The present inventors have also determined biomarker panels that are particularly relevant for males and females.

In a second aspect, the present disclosure provides a method for diagnosing colorectal cancer and/or identifying a subject suspected of having, or at a greater risk of having colorectal cancer, wherein the subject is female, the method comprising:
determining a measurement for a panel of biomarkers in a biological sample obtained from the subject, the panel comprising at least brain derived neurotropic factor (BDNF) and tumour M2-PK
wherein the measurement comprises measuring a level of each of the biomarkers in the panel.

In one example according to the second aspect, the method comprises determining a measurement for a panel of at least three biomarkers, wherein the panel comprises at least BDNF and M2PK and a further biomarker selected from the group consisting of TNFα, IGFBP2, TIMP1, MIP1β, MMP7, MMP1, IGFII, M65, M30, LIPOCALIN, IL8, IL13, MAC2BP, TGFβ1 and IL6, preferably selected from the group consisting of MIP1β, MMP1, LIPOCALIN, IL13, IL8, MAC2BP, and IL6.

In another example according to the second aspect, the three biomarker panels comprise or consist of:
(i) BDNF; M2PK; IL8; ii) BDNF; M2PK; MAC2BP; iii) BDNF; M2PK; MMP1; iv). BDNF; M2PK; LIPOCALIN; v) BDNF; M2PK; IL13; vi) BDNF; M2PK; MIP1β; or vii) BDNF; M2PK; IL6.

In another example according to the second aspect, the method comprises determining a measurement for a panel of at least four biomarkers, wherein the panel comprises at least BDNF and M2PK and two biomarkers selected from the group consisting of TNFα, IGFBP2, TIMP1, MIP1β, MMP7, MMP1, IGFII, M65, M30, LIPOCALIN, IL8, IL13, MAC2BP, TGFBETA1 and IL6, preferably selected from the group consisting of TNFα, MIP1β, MMP1, IGFII, LIPOCALIN, IL8, IL13, MAC2BP, TGFβ1 and IL6.

In another example according to the second aspect, the four biomarker panels comprise or consist of:
i) BDNF, M2PK, IL8, MMP1; ii) BDNF, M2PK, IL8, LIPOCALIN; iii) BDNF, M2PK, IL8, IL6; iv) BDNF, M2PK, IL8, MIP1β; v) BDNF, M2PK, IL8, MAC2BP; vi) BDNF, M2PK, IL8, TGFβ; vii) BDNF, M2PK, IL8, IL13; viii) BDNF, M2PK, IL8, IGFII; ix) BDNF, M2PK, IL8, TNFα; or x). BDNF, M2PK, TGFβ1, MMP1.

In another example according to the second aspect, the method comprises determining a measurement for a panel of at least five biomarkers, wherein the panel comprises at least BDNF and M2PK and three biomarkers selected from the group consisting of TNFα, IGFBP2, TIMP1, MIP1β, MMP7, MMP1, IGFII, M65, M30, LIPOCALIN, IL13, MAC2BP, TGFβ1 and IL6, preferably selected from the group consisting of TNFα, MIP1β, MMP1, IGFII, IL8, IL13, MAC2BP, TGFβ1 and IL6.

In another example according to the second aspect, the method comprises determining a measurement for a panel of at least five biomarkers, wherein the panel comprises at least BDNF, M2PK and IL-8 and two biomarkers selected from the group consisting of TNFα, IGFBP2, TIMP1, MIP1β, MMP7, MMP1, IGFII, M65, M30, LIPOCALIN, IL13, MAC2BP, TGFβ1 and IL6, preferably selected from the group consisting of TNFα, MIP1β, MMP1, IGFII, LIPOCALIN, IL13, MAC2BP, TGFβ1 and IL6.

In another example according to the second aspect, the five biomarker panels comprise or consist of:
i) BDNF, M2PK, IL8, TGFβ1, TNFα; ii) BDNF, M2PK, IL8, TNFα, MMP1; iii). BDNF, M2PK, IL8, TGFβ1, MMP1; iv) BDNF, M2PK, IL8, TGFβ1, MAC2BP; v) BDNF, M2PK, IL8, TGFβ; vi) BDNF, M2PK, IL8, TGFβ1, IL13; vii) BDNF, M2PK, IL8, TGFβ1, IGFII; viii) BDNF, M2PK, IL8, TGFβ1, MIP1β; ix) BDNF, M2PK, IL8, MMP1, MIP1β, or x) BDNF, M2PK, IL8, MAC2BP, MIP1β.

In another example according to the second aspect, the method comprises determining a measurement for a panel of at least six biomarkers, wherein the panel comprises at least BDNF and M2PK and four biomarkers selected from the group consisting of TNFα, IGFBP2, TIMP1, MIP1β, MMP7, MMP1, IGFII, M65, M30, LIPOCALIN, IL13, MAC2BP, TGFβ1 and IL6, preferably selected from the group consisting of DKK3, TNFα, MIP1β, MMP1, M65, M30, IL8, IL13, MAC2BP, TGFβ1 and IL6.

In another example according to the second aspect, the six biomarker panels comprise or consist of:
i). BDNF, M2PK, IL8, IL13, M65, M30; ii) BDNF, M2PK, IL8, MMP1, TGFβ1, TNFα; iii) BDNF, M2PK, IL8, MAC2BP, TGFβ1, TNFα; iv) BDNF, M2PK, IL8, IL6, TGFβ1, TNFα; v) BDNF, M2PK, IL8, MMP1, MAC2BP, TNFα; vi) BDNF, M2PK, IL8, MMP1, TGFβ1, MAC2BP; vii) BDNF, M2PK, IL8, MMP1, TGFβ1, DKK3; viii) BDNF, M2PK, IL8, IL6, TGFβ1, MAC2BP; ix). BDNF, M2PK, IL8, MMP1, DKK3, MAC2BP, or x) BDNF, M2PK, IL8, IL13, TGFβ1, MIP1β.

In another example according to the second aspect, the method comprises determining a measurement for a panel of at least seven biomarkers, wherein the panel comprises at least BDNF and M2PK and five or more biomarkers selected from the group consisting of IL8, M65, MMP1, IL13, IGFBP2, TNFA, MIP1B, LIPOCALIN, MAC2BP, IL6, MMP7, IGFII, TGFβ1 and M30. In one example, the method comprises determining a measurement for a panel of at least seven biomarkers, wherein the panel comprises at least BDNF, M2PK and IL8, and four or more biomarkers selected from the group consisting of M65, MMP1, IL13, IGFBP2, TNFA, MIP1B, LIPOCALIN, MAC2BP, IL6, MMP7, IGFII, TGFβ1 and M30. In one example, the method comprises determining a measurement for a panel of at least seven biomarkers, wherein the panel comprises at least BDNF, M2PK, M65 and IL8, and three or more biomarkers selected from the group consisting of MMP1, IL13, IGFBP2, TNFA, MIP1B, LIPOCALIN, MAC2BP, IL6, MMP7, IGFII, TGFβ1 and M30. In one example, the method comprises determining a measurement for a panel of at least seven biomarkers, wherein the panel comprises at least BDNF, M2PK, M65, MMP1 and IL8, and two or more biomarkers selected from the group consisting of IL13, IGFBP2, TNFA, MIP1B, LIPOCALIN, MAC2BP, IL6, MMP7, IGFII, TGFβ1 and M30.

In one example, according to the second aspect, the method comprises determining a measurement for a panel of biomarkers present in Table 28.

In another example according to the second aspect, the method comprises determining a measurement for a panel of at least eight biomarkers, wherein the panel comprises at least BDNF and M2PK and six or more biomarkers selected from the group consisting of IL8, M65, MMP1, TNFA, MIP1B, LIPOCALIN, MAC2BP, IL6, MMP7, IGFII, TIMP1, TGFβ1, M30 and DKK3. In one example, the method comprises determining a measurement for a panel of at least eight biomarkers, wherein the panel comprises at least BDNF, M2PK and IL8 and five or more biomarkers selected from the group consisting of M65, MMP1, TNFA, MIP1B, LIPOCALIN, MAC2BP, IL6, MMP7, IGFII, TIMP1, TGFβ1, M30 and DKK3. In one example, the method comprises determining a measurement for a panel of at least eight biomarkers, wherein the panel comprises at least BDNF, M2PK, IL8 and M65 and four or more biomarkers selected from the group consisting of MMP1, TNFA, MIP1B, LIPOCALIN, MAC2BP, IL6, MMP7, IGFII, TIMP1, TGFβ1, M30 and DKK3. In one example, the method comprises determining a measurement for a panel of at least eight biomarkers, wherein the panel comprises at least BDNF, M2PK, IL8, M65 and MMP1 and three or more biomarkers selected from the group consisting of TNFA, MIP1B, LIPOCALIN, MAC2BP, IL6, MMP7, IGFII, TIMP1, TGFβ1, M30 and DKK3.

In one example, according to the second aspect, the method comprises determining a measurement for a panel of biomarkers present in Table 27, 26 or 25.

In another example according to the second aspect, the method further comprises the subjects age as an additional biomarker. In one example the method comprises determining a measurement for a panel of biomarkers present in Table 34, 35, 36, 37, 38, 39, 40, 41, or 42.

In a third aspect, the present disclosure provides a method for diagnosing colorectal cancer and/or identifying a subject suspected of having, or at a greater risk of having colorectal cancer, wherein the subject is male, the method comprising:
determining a measurement for a panel of biomarkers in a biological sample obtained from the subject, the panel comprising at least brain derived neurotropic factor (BDNF) and tumour M2-PK
wherein the measurement comprises measuring a level of each of the biomarkers in the panel.

In one example according to the third aspect, the method comprises determining a measurement for a panel of at least three biomarkers, wherein the panel comprises at least BDNF and M2PK and a further biomarker selected from the group consisting of DKK3, TNFA, IGFBP2, TIMP1, MIP1β, MMP7, MMP1, IGFII, M65, M30, LIPOCALIN, IL8, IL13, MAC2BP and TGFβ1, preferably selected from the group consisting of TNFA, IGFBP2, TIMP1, MMP7, IGFII, LIPOCALIN, IL8, IL13, MAC2BP and TGFβ1.

In another example according to the third aspect, the three biomarker panels comprise or consist of:
i) BDNF, M2PK, TNFα; ii) BDNF, M2PK, TIMP1; iii) BDNF, M2PK, IL8; iv) BDNF, M2PK, TGFβ; v) BDNF, M2PK, MMP7; vi) BDNF, M2PK, IL13; vii) BDNF, M2PK, IGFII; viii) BDNF, M2PK, IGFBP2; ix) BDNF, M2PK, MAC2BP, or x) BDNF, M2PK, LIPOCALIN.

In another example according to the third aspect, the method comprises determining a measurement for a panel of at least four biomarkers, wherein the panel comprises at least BDNF and M2PK and two biomarkers selected from the group consisting of DKK3, TNFα, IGFBP2, TIMP1, MIP1β, MMP7, MMP1, IGFII, M65, M30, LIPOCALIN, IL8, IL13, MAC2BP and TGFβ1, preferably selected from the group consisting of TNFα, IGFBP2, TIMP1, MIP1B, MMP7, M65 and IL8.

In another example according to the third aspect, the four biomarker panels comprise or consist of:
i) BDNF, M2PK, DKK3, TIMP1; ii) BDNF, M2PK, IL8, TNFα; iii) BDNF, M2PK, IGFBP2, TNFα; iv) BDNF, M2PK, IL8, IGFBP2; v) BDNF, M2PK, DKK3, TNFα; vi) BDNF, M2PK, IL8, MMP7; vii) BDNF, M2PK, DKK3, M65; viii) BDNF, M2PK, DKK3, IL8; ix) BDNF, M2PK, DKK3, IGFBP2, or x) BDNF, M2PK, MIP1β, TIMP1.

In another example according to the third aspect, the method comprises determining a measurement for a panel of at least five biomarkers, wherein the panel comprises at least BDNF and M2PK and three biomarkers selected from the group consisting of DKK3, TNFα, IGFBP2, TIMP1, MIP1β, MMP7, MMP1, IGFII, M65, M30, LIPOCALIN, IL8, IL13, MAC2BP and TGFβ1, preferably selected from the group consisting of DKK3, TNFA, IGFBP2, TIMP1, MIP1β, IGFII, M65, LIPOCALIN, IL8 and MAC2BP.

In another example according to the third aspect, the five biomarkers comprise or consist of:
i) BDNF; M2PK; DKK3; IGFBP2; TNFα; ii) BDNF; M2PK; DKK3; LIPOCALIN; TIMP1; iii) BDNF; M2PK; DKK3; M65; TNFα; iv) BDNF; M2PK; DKK3; IGFBP2; TIMP1; v) BDNF; M2PK; DKK3; IL8; IGFII; vi) BDNF; M2PK; IL13; IL8; TNFα; vii) BDNF; M2PK; DKK3; MAC2BP; TIMP1; viii) BDNF; M2PK; DKK3; M65; TIMP1; ix) BDNF; M2PK; DKK3; MIP1B; TNFα; or x) BDNF; M2PK; DKK3; MIP1B; TIMP1.

In another example according to the third aspect, the method comprises determining a measurement for a panel of at least five biomarkers, wherein the five biomarkers comprise BDNF, M2PK, DKK-3 and two biomarkers selected from TNFα, IGFBP2, TIMP1, MIP1β, MMP7, MMP1, IGFII, M65, M30, LIPOCALIN, IL8, IL13, MAC2BP or TGFBETA1, preferably selected from TNFα, IGFBP2, TIMP1, MIP1β, IGFII, M65, LIPOCALIN, IL8 or MAC2BP.

In one example according to the third aspect, the five biomarkers comprise or consist of:
i) BDNF; M2PK; DKK3; IGFBP2; TNFα; ii) BDNF; M2PK; DKK3; LIPOCALIN; TIMP1; iii) BDNF; M2PK; DKK3; M65; TNFα; iv) BDNF; M2PK; DKK3; IGFBP2; TIMP1; v) BDNF; M2PK; DKK3; IL8; IGFII; vi) BDNF; M2PK; DKK3; MAC2BP; TIMP1; vii) BDNF; M2PK; DKK3; M65; TIMP1; viii). BDNF; M2PK; DKK3; MIP1β; TNFα; or ix) BDNF; M2PK; DKK3; MIP1β; TIMP1.

In another example according to the third aspect, the method comprises determining a measurement for a panel of at least six biomarkers, wherein the six biomarkers comprise BDNF and M2PK and four biomarkers selected from the group consisting of DKK3, TNFα, IGFBP2, TIMP1, MIP1β, MMP7, MMP1, IGFII, M65, M30, LIPOCALIN, IL8, IL13, MAC2BP and TGFβ1, preferably selected from the group consisting of DKK3, TNFα, IGFBP2, TIMP1, MIP1β, MMP7, MMP1, IGFII, M65, M30, LIPOCALIN, IL8 and IL13.

In another example according to the third aspect, the biomarkers comprise BDNF, M2PK, DKK3, TNFα and two biomarkers selected from the group consisting of IGFBP2, TIMP1, MIP1β, MMP7, MMP1, IGFII, M65, M30, LIPOCALIN, IL8, IL13, MAC2BP and TGFβ1, preferably selected from the group consisting of IGFBP2, TIMP1, MIP1β, MMP7, MMP1, IGFII, M65, M30, LIPOCALIN, IL8 and IL13.

In one example according to the third aspect the six biomarkers comprise or consists of:
i) BDNF, M2PK, DKK3, TNFα, TIMP1, IL8; ii) BDNF, M2PK, DKK3, TNFα, IGFBP2, MMP1; iii) BDNF, M2PK, DKK3, TNFα, IGFBP2, M30; iv) BDNF, M2PK, DKK3, TNFα, MIP1B, M65; v) BDNF, M2PK, DKK3, TNFα, IGFBP2, MMP7; vi) BDNF, M2PK, DKK3, TNFα, IGFBP2, M65; vii) BDNF, M2PK, DKK3, TNFα, IGFBP2, LIPOCALIN; viii) BDNF, M2PK, DKK3, TNFα, IGFII, IL8; ix) BDNF, M2PK, DKK3, TNFα, M65, M30, or x) BDNF, M2PK, DKK3, TNFα, IGFBP2, IL13.

In another example according to the third aspect, the method comprises determining a measurement for a panel of at least seven biomarkers, wherein the panel comprises at least BDNF, M2PK and five or more biomarkers selected from the group consisting of DKK3, TNFα, MIP1β, MMP1, IGFBP2, LIPOCALLIN, TIMP1, M30, IGII, IL8, MMP7 and M65. In one example, the method comprises determining a measurement for a panel of at least seven biomarkers, wherein the panel comprises at least BDNF, M2PK, DKK3, and four or more biomarkers selected from the group consisting of TNFα, MIP1β, MMP1, IGFBP2, LIPOCALLIN, TIMP1, M30, IGII, IL8, MMP7 and M65. In one example, the method comprises determining a measurement for a panel of at least seven biomarkers, wherein the panel comprises at least BDNF, M2PK, DKK3, TNFα, and three or more biomarkers selected from the group consisting of MIP1β, MMP1, IGFBP2, LIPOCALLIN, TIMP1, M30, IGII, IL8, MMP7 and M65. In one example, the method comprises determining a measurement for a panel of at least seven biomarkers, wherein the panel comprises at least BDNF, M2PK, DKK3, TNFα, IGFBP2, and two or more biomarkers selected from the group consisting of MIP1β, MMP1, LIPOCALLIN, TIMP1, M30, IGII, IL8, MMP7 and M65.

In another example, according to the third aspect, the method comprises determining a measurement for a panel of biomarkers present in Table 46.

In another example according to the third aspect, the method comprises determining a measurement for a panel of at least eight biomarkers, wherein the panel comprises at least BDNF, M2PK and six or more biomarkers selected from the group consisting of DKK3, TNFα, MIP1β, MMP1, IGFBP2, LIPOCALLIN, TIMP1, TGFβ1, M30, IL13, IGII, IL8, MMP7 and IL13. In one example, the method comprises determining a measurement for a panel of at least eight biomarkers, wherein the panel comprises at least BDNF, M2PK, DKK3 and five or more biomarkers selected from the group consisting of TNFα, MIP1β, MMP1, IGFBP2, LIPOCALLIN, TIMP1, TGFβ1, M30, IL13, IGII, IL8, MMP7 and IL13. In one example, the method comprises determining a measurement for a panel of at least eight biomarkers, wherein the panel comprises at least BDNF, M2PK, DKK3, TNFα and four or more biomarkers selected from the group consisting of MIP1β, MMP1, IGFBP2, LIPOCALLIN, TIMP1, TGFβ1, M30, IL13, IGII, IL8, MMP7 and IL13. In one example, the method comprises determining a measurement for a panel of at least eight biomarkers, wherein the panel comprises at least BDNF, M2PK, DKK3, TNFα, MMP7 and three or more biomarkers selected from the group consisting of MIP1β, MMP1, IGFBP2, LIPOCALLIN, TIMP1, TGFβ1, M30, IL13, IGII, IL8 and IL13.

In another example, according to the third aspect, the method comprises determining a measurement for a panel of biomarkers present in Table 45.

In another example, the method according to the third aspect comprises determining a measurement of at least nine, or at least ten biomarkers, wherein the biomarkers comprise at least BDNF and M2PK. In one example the method comprises determining a measurement for a panel of biomarkers present in Table 44 or 43.

In a further example, the biomarkers comprise PKM2 (also referred to as M2PK), BDNF, DKK3, IGFBP2 and TIMP1.

In another example according to the third aspect, the method further comprises the subjects age as an additional biomarker. In one example the method comprises determining a measurement for a panel of biomarkers present in Table 52, 53, 54, 55, 56, 57, 58, 59 or 60.

In a further example, the methods described herein may use one or more or all of the biomarker combinations provided in Tables 7 to 15, Tables 16 to 24, Tables 25 to 33, Tables 34 to 42, Tables 43 to 51, Tables 52 to 60 or Tables 61 to 66.

In a fourth aspect, there is provided a biomarker combination set forth in any one of Tables 7 to 15, Tables 16 to 24, Tables 25 to 33, Tables 34 to 42, Tables 43 to 51, Tables 52 to 60 or Tables 61 to 66.

It will be understood that the present disclosure encompasses additional known colorectal cancer biomarkers which are not specifically described herein. Examples of these additional biomarkers include one or more of IGF-I, Amphiregulin, VEGFA, VEGFD, MMP2, MMP3, MMP9, TIMP2, ENA-78, MCP-1, IFN-γ, IL10, IL-1B, IL4, OPN, CEACAM6, VEGFalpha and VEGFpan.

It will be understood that, in addition to age and/or gender, the present disclosure encompasses additional demographic or morphometric terms which are not specifically described herein. Examples of these other demographic or morphometric terms include, but not limited to, smoking history, body mass index (BMI) and hip to waist ratio.

In one example according to any aspect described herein, determining a measurement comprises measuring the concentration of the biomarker in the biological sample. In one example according to any aspect, determining a measurement comprises detecting biomarkers in the biological sample by contacting the sample with detectable binding agents that specifically bind to the biomarkers. In a further example according to any aspect, the method comprises detecting specific binding between the specific binding agents and the biomarkers using a detection assay. In a further example according to any aspect, determining a measurement comprises performing a statistical analysis.

In one example according to any aspect described herein, the method comprises a capture antibody. In one example, the capture antibody is immobilised, for example on a plate. In one example, the plate is an ELISA plate. In one example according to any aspect, the method comprises a labelled antibody for detecting binding of the biomarker to the capture antibody.

In one example according to any aspect, the level of at least one biomarker in the panel of biomarkers is increased or decreased relative to a level of the same biomarker in a reference panel. More particularly, the measurement of a biomarker is relative to a reference concentration for that biomarker determined in known cases of CRC and/or control samples by an algorithm trained on the case and control samples.

In one example according to any aspect, the methods of the disclosure comprise:
(i) performing a measurement of the concentration of each biomarker in a biomarker panel described herein to derive a statistical value;
(ii) comparing the value obtained in step (i) to a statistical value obtained from the concentration of the same biomarkers in a corresponding biomarker reference panel; and
(iii) correlating the value obtained in step (ii) to derive a value determinative of the colorectal cancer risk of the subject.

In some examples, the biomarkers are protein biomarkers. In one example, the biomarkers are polynucleotide biomarkers.

In some examples, the methods of the disclosure comprise contacting the biological sample with antibodies that specifically bind to the biomarker proteins. Preferably, there is at least one antibody that binds individually to each biomarker sought to be detected in the biological sample. Preferably, the antibodies specifically bind to a given biomarker. In some examples, more than one antibody may bind to a single biomarker, for example in a “sandwich” format.

In some examples, the measuring format is an immunoassay. In another example, the immunoassay is an ELISA, typically there would be a capture antibody bound to the surface of the ELISA plate and a detection antibody to detect binding of the biomarker to the capture antibody. In one example, the antibody may be detectably labelled. In one example the capture antibody may be the same antibody or a different antibody to the detection antibody. Methods of labelling antibodies are known in the art.

In some examples, if the biomarkers are polynucleotides, then the analysis method may comprise measuring a gene transcript corresponding to an individual biomarker. Such methods will be familiar to those skilled in the art.

Methods of performing model building and statistical analysis will be known to persons skilled in the art. In some examples, linear or non-linear regression is performed. The methods may also utilise a Bayesian probability algorithm.

In some examples, the analysis of the biomarker panel may be used to determine a treatment regimen for the subject. For example, the statistical value obtained from the measurement of the biomarkers may be used to inform further treatment by colonoscopy or sigmoidoscopy to provide a definitive diagnosis of colorectal cancer.

In a fifth aspect, there is provided method of treating a subject suspected of having colorectal cancer, the method comprising:
(i) determining a measurement for a panel of biomarkers in a biological sample obtained from the subject according to a method described herein; and
(ii) treating the subject by colonoscopy or sigmoidoscopy.

In another example according to any aspect, the method further describes obtaining a biological sample from the subject. In a further example, the biological sample is a blood sample. In another example, the sample is a serum or plasma sample. Methods of obtaining a biological sample will be known to those skilled in the art. For example for the extraction of a blood sample, it is preferred that a venous draw is performed.

In a sixth aspect, there is provided a composition comprising labelled antibodies that specifically bind to the biomarkers in a biomarker panel described herein.

In a seventh aspect, there is provided a kit comprising:
(i) labelled antibodies that bind to the biomarkers in a biomarker panel described herein; and
(ii) instructions for performing detection of the biomarkers in the biomarker panel.

In some examples, the kit also comprises a surface on which is immobilised capture antibodies which bind to each biomarker in the biomarker panel. In some examples, the kit also comprises an ELISA plate on which is immobilised capture antibodies which bind to each biomarker in the biomarker panel. In some examples, the kit also comprises a bead (e.g. microbead or magnetic bead) on which is immobilised capture antibodies which bind to each biomarker in the biomarker panel. In some examples, the kit also provides instructions for the analysis of the detected biomarkers by a computer generated algorithm. In a further example, a clinical report is generated.

In an eighth aspect, there is provided a kit as described herein together with a software package comprising an algorithm for generating a statistical value based on the measurement of biomarkers in a biomarker panel described herein.

In a ninth aspect, there is provided a system for determining a subject suspect of, or at a greater risk of, colorectal cancer, comprising
(i) determining a measurement for a panel of biomarkers as described herein in a biological sample obtained from the subject, wherein the measurement is the concentration of each biomarker in the panel,
(ii) inputting the concentration of each biomarker in the panel into a computer comprising an input device, operatively connected to the computer to receive the concentration of each biomarker in the panel;
(iii) an output device connected to the computer to provide information to a user; and
(iv) an algorithm executed by the computer wherein the algorithm is executed based on the data received by the input device, and wherein the algorithm calculates a risk score.

### FIGURES

FIGS. 1-1 and 1-2 (A) shows the frequency with which each biomarker appears in the top 100 ten-biomarker combinations differentiating between sera from cancer patients and those of healthy controls with sensitivities ranging from 85.6-82.2% at 95% specificity. (B) shows the frequency with which each biomarker appears in the top 100 nine-biomarker combinations differentiating between sera from cancer patients and those of healthy controls with sensitivities ranging from 84.7-81.7% at 95% specificity. (C) The frequency with which each biomarker appears in the top 100 eight-biomarker combinations differentiating between sera from cancer patients and those of healthy controls with sensitivities ranging from 84.2-80.6% at 95% specificity. (D) The frequency with which each biomarker appears in the top 100 seven-biomarker combinations differentiating between sera from cancer patients and those of healthy controls with sensitivities ranging from 83.2-79.2% at 95% specificity. (E) The frequency with which each biomarker appears in the top 100 six-biomarker combinations differentiating between sera from cancer patients and those of healthy controls with sensitivities ranging from 80.4-77.0% at 95% specificity. (F) The frequency with which each biomarker appears in the 74 five-biomarker combinations differentiating between sera from cancer patients and those of healthy controls with sensitivities>75% (Range, 80.5-75.0%) at 95% specificity.

FIGS. 2-1 and 2-2 (A) shows the frequency with which each biomarker appears in the top 100 ten-biomarker combinations (plus age) differentiating between sera from cancer patients and those of healthy controls with sensitivities ranging from 86.8% to 83.5% at 95% specificity. (B) shows the frequency with which each biomarker appears in the top 100 nine-biomarker combinations (plus age) differentiating between sera from cancer patients and those of healthy controls with sensitivities ranging from 87.0% to 82.9% at 95% specificity. (C) The frequency with which each biomarker appears in the top 100 eight-biomarker combinations (plus age) differentiating between sera from cancer patients and those of healthy controls with sensitivities ranging from 85.1% to 81.7% at 95% specificity. (D) The frequency with which each biomarker appears in the top 100 seven-biomarker combinations (plus age) differentiating between sera from cancer patients and those of healthy controls with sensitivities ranging from 84.2% to 79.9% at 95% specificity. (E) The frequency with which each biomarker appears in the top 100 six-biomarker combinations (plus age) differentiating between sera from cancer patients and those of healthy controls with sensitivities ranging from 83.2% to 77.5% at 95% specificity. (F) The frequency with which each biomarker appears in the 73 five-biomarker combinations (plus age) differentiating between sera from cancer patients and those of healthy controls with sensitivities>75% (Range, 80.0-75.0%) at 95% specificity.

FIGS. 3-1 and 3-2 (A) The frequency with which each biomarker appears in the top 100 ten-biomarker combinations differentiating between sera from female cancer patients and those of healthy controls at 95% specificity. (B) The frequency with which each biomarker appears in the top 100 nine-biomarker combinations differentiating between sera from female cancer patients and those of healthy controls at 95% specificity. (C) The frequency with which each biomarker appears in the top 100 eight-biomarker combinations differentiating between sera from female cancer patients and those of healthy controls at 95% specificity. (D) The frequency with which each biomarker appears in the top 100 seven-biomarker combinations differentiating between sera from female cancer patients and those of healthy controls at 95% specificity. (E) The frequency with which each biomarker appears in the top 100 six-biomarker combinations differentiating between sera from female cancer patients and those of healthy controls at 95% specificity. (F) The frequency with which each biomarker appears in the top 100 five-biomarker combinations differentiating between sera from female cancer patients and those of healthy controls at 95% specificity.

FIGS. 4-1 and 4-2 (A) The frequency with which each biomarker appears in the top 100 ten-biomarker combinations (plus age) differentiating between sera from female cancer patients and those of healthy controls at 95% specificity. (B) The frequency with which each biomarker appears in the top 100 nine-biomarker combinations (plus age) differentiating between sera from female cancer patients and those of healthy controls at 95% specificity. (C) The frequency with which each biomarker appears in the top 100 eight-biomarker combinations (plus age) differentiating between sera from female cancer patients and those of healthy controls at 95% specificity. (D) The frequency with which each biomarker appears in the top 100 seven-biomarker combinations (plus age) differentiating between sera from female cancer patients and those of healthy controls at 95% specificity. (E) The frequency with which each biomarker appears in the top 100 six-biomarker combinations (plus age) differentiating between sera from female cancer patients and those of healthy controls at 95% specificity. (F) The frequency with which each biomarker appears in the top 100 five-biomarker combinations differentiating between sera from female cancer patients and those of healthy controls at 95% specificity.

FIGS. 5-1 and 5-2 (A) The frequency with which each biomarker appears in the top 100 ten-biomarker combinations differentiating between sera from male cancer patients and those of healthy controls at 95% specificity. (B) The frequency with which each biomarker appears in the top 100 nine-biomarker combinations differentiating between sera from male cancer patients and those of healthy controls at 95% specificity. (C) The frequency with which each biomarker appears in the top 100 eight-biomarker combinations differentiating between sera from male cancer patients and those of healthy controls at 95% specificity. (D) The frequency with which each biomarker appears in the top 100 seven-biomarker combinations differentiating between sera from male cancer patients and those of healthy controls at 95% specificity. (E) The frequency with which each biomarker appears in the top 100 six-biomarker combinations differentiating between sera from male cancer patients and those of healthy controls at 95% specificity. (F) The frequency with which each biomarker appears in the top five-biomarker combinations differentiating between sera from female cancer patients and those of healthy controls with sensitivities>75% at 95% specificity.

FIGS. 6-1 and 6-2 (A) The frequency with which each biomarker appears in the top 100 ten-biomarker combinations (plus age) differentiating between sera from male cancer patients and those of healthy controls at 95% specificity. (B) The frequency with which each biomarker appears in the top 100 nine-biomarker combinations (plus age) differentiating between sera from male cancer patients and those of healthy controls at 95% specificity. (C) The frequency with which each biomarker appears in the top 100 eight-biomarker combinations (plus age) differentiating between sera from male cancer patients and those of healthy controls at 95% specificity. (D) The frequency with which each biomarker appears in the top 100 seven-biomarker combinations (plus age) differentiating between sera from male cancer patients and those of healthy controls at 95% specificity. (E) The frequency with which each biomarker appears in the top 100 six-biomarker combinations (plus age) differentiating between sera from male cancer patients and those of healthy controls at 95% specificity. (F) The frequency with which each biomarker appears in the top five-biomarker combinations (plus age) differentiating between sera from female cancer patients and those of healthy controls with sensitivities>75% at 95% specificity.

FIG. 7 shows training and testing structures for study 9. This figure represents an example of the train test makeups (i.e. cohort 2 was also used to train algorithms and cohort 1 was used to test).

### DETAILED DESCRIPTION

### General Techniques and Definitions

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, 3^{rd} edn, Cold Spring Harbour Laboratory Press (2001), R. Scopes, Protein Purification—Principals and Practice, 3^{rd} edn, Springer (1994), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

“Colorectal cancer (CRC)” as used herein refers to cancer that starts in the colon or rectum. These cancers can also be referred to separately as colon cancer or rectal cancer, depending on where they start. Colon cancer and rectal cancer have many features in common. More than 95% of colorectal cancers are a type of cancer known as adenocarcinomas. These cancers start in cells derived from glands that make mucus to lubricate the inside of the colon and rectum. In most cases these cells first form benign outgrowths of the colorectal epithelium called adenomas and over 90% of colorectal cancers first appear as small foci of highly dysplastic tissue within these otherwise benign adenomas. Other, less common types of tumours may also start in the colon and rectum. These include: carcinoid tumours, gastrointestinal stromal tumours (GISTs), lymphomas and sarcomas. In a preferred example, said colorectal cancer is adenocarcinoma. Adenocarcinomas are staged to help guide clinical management. The staging system most often used for colorectal cancer is the American Joint Committee on Cancer (AJCC) TNM system (https://www.cancer.org/cancer/colon-rectal-cancer/detection-diagnosis-staging/staged.html), which is based on 3 key pieces of information:
T—The size of the tumor (Levels 0-4):
   How far has the cancer grown into the wall of the colon or rectum: Is it still confined to the colonic epithelium? Has it penetrated the thin muscle layer directly below the epithelium? Has it penetrated the fibrous tissue beneath this thin muscle layer? Has it penetrated a thicker muscle layer below? Has it penetrated the layer of connective tissue below the major muscle layer?
N—The level of lymph node involvement (Levels 0-2): Has the cancer spread to nearby lymph nodes? If so, how many?
M—The level of metastatic spread (Levels 0-1): Has the cancer spread to distant lymph nodes or distant organs such as the liver or lungs?

The term, “biomarker” as used herein, refers to any biological compound that can be measured as an indicator of the physiological status of a biological system. In some examples, the biomarker is a polynucleotide or nucleic acid. In some examples, the biomarker is a polypeptide or protein. A biomarker can also be a subject's age, gender and/or BMI as described further herein.

The term “measurement” as used herein refers to assessing the presence, absence, quantity or amount of a given substance within a sample, including the derivation of qualitative or quantitative concentration levels of such substances. The term “measuring” means methods which include detecting the presence or absence of biomarker(s) in the sample, quantifying the amount of biomarker(s) in the sample, and/or qualifying the type of biomarker. Measuring can be accomplished by methods known in the art and those further described herein, including but not limited to mass spectrometry approaches and immunoassay approaches (e.g. ELISA) or any suitable methods can be used to detect and measure one or more of the markers described herein. Reference to the biomarker sequences can be found in Table 2 which provides the UniProt (uniprot.org) and NCBI/Genbank accession numbers (ncbi.nlm.nih.gov/genbank).

The term “detect” refers to identifying the presence, absence or amount of the biomarker to be detected. Non-limiting examples include, but are not limited to, detection of, proteins, peptides, or nucleic acids.

The term “report” refers to a printed result provided from the methods of the present invention to the physician. The report can indicate the presence of, nature of, or risk for the pathological condition. The report can also indicate what treatment is most appropriate e.g. no action, surgery, further tests, or administering a therapeutic agent.

As used herein, the term “biological sample” refers to a cell or population of cells or a quantity of tissue or fluid from a subject. Most often, the sample has been removed from a subject, but the term “biological sample” can also refer to cells or tissue analyzed in vivo, i.e. without removal from the subject. Preferably, a “biological sample” refers to non-cellular fractions of blood, saliva, or urine. Biological samples include, but are not limited to whole blood, plasma, serum, lymph, or urine.

The term “control reference” as used herein refers to a known steady state molecule or a non-diseased, healthy condition that is used as a relative marker in which to study fluctuations or compare the non-steady state molecules or normal non-diseased healthy condition, or it can also be used to calibrate or normalise values. In some examples, a control reference value is a calculated value such as a combination of biomarker concentrations or a combination of ranges of concentrations.

The term “immunoassay” is an assay that uses an antibody to specifically bind an antigen (e.g., a marker). The immunoassay is characterized by the use of specific binding properties of a particular antibody to isolate, target, and/or quantify the antigen.

The term “antibody” refers to a polypeptide ligand substantially encoded by an immunoglobulin gene or immunoglobulin genes, or fragments thereof, which specifically binds and recognizes an epitope. Antibodies exist, e.g., as intact immunoglobulins or as a number of well-characterized fragments produced by digestion with various peptidases. This includes, e.g., Fab″ and F(ab)″2 fragments. As used herein, the term “antibody” also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, but does not include the heavy chain variable region.

As used herein, the term “subject” refers to any animal that may develop colorectal cancer and includes animals such as mammals, e.g. humans, or non-human mammals such as cats and dogs, laboratory animals such as mice, rats, rabbits or guinea pigs, and livestock animals. In a preferred embodiment, the subject is a human.

The term “sample” or “biological sample” as used herein refers to a sample of biological fluid, tissue, or cells in a healthy and/or pathological state obtained from a subject. Preferably, the term “sample” is a blood sample, more preferably a serum sample.

### General Overview

The present disclosure provides methods for the analysis of a biological sample from a subject using an assay coupled with an algorithm executable by a computer for determining biomarkers which are indicative of colorectal cancer. Generally, the methods use proteins present in the biological sample of the subject to identify biomarkers or a biomarker profile and thus identify subjects who have colorectal cancer or are at a higher risk for colorectal cancer and who may require further screening such as colonoscopy or sigmoidoscopy.

The present disclosure also provides a commercial diagnostic kit that in general will include compositions used for the detection of biomarkers provided herein.

### Biomarkers

The present disclosure utilises a panel of biomarkers measured in a biological sample obtained from a subject to identify subjects that have, or are suspected of having colorectal cancer.

The term ‘biomarker” as used herein, refers to any biological compound that can be measured as an indicator of the physiological status of a biological system. In some examples, the biomarker is a protein biomarker. In other examples, the biomarker is a nucleic acid biomarker.

The present studies have demonstrated a particular role for brain derived neurotrophic factor (BDNF) as an informative biomarker for identifying subjects at increased risk of colorectal cancer. BDNF has been observed to be elevated in solid tumours including colorectal cancer (Yang X et al., (2013) Exp Ther Med 6(6):1475-1481). Moreover, when this biomarker is combined with M2PK, a known marker in CRC, sensitivity of detection is comparable to or greater than that achieved with the fecal occult blood test (FOBT). According to the Cancer Council of Australia, the sensitivity of FOBT for advanced adenoma ranges from 16-64% at around 93% specificity (see https://wiki.cancer.org.au/policy/Bowel_cancer/Screening).

In some examples, the biomarker panel may include 2, 3, 4, 5, 6 or more biomarkers selected from the group consisting of DKK3, M2PK, TGFβ, IGFBP2, TIMP1, BDNF, IL6, IL8, TNFα, IGFII, Lipocalin, M30, M65, Mac2BP, MMP1, MMP7, MIP1β, and IL13.

Reference to any of these biomarkers includes reference to all polypeptide and polynucleotide variants such as isoforms and transcript variants as would be known by the person skilled in the art. NCBI accession numbers of representative sequences for each of the biomarkers are provided in Table 1 of the Examples.

It will be understood that, in some examples, demographic or morphometric terms may also be factored into the analysis, for example, logistic regression algorithm. Demographic or morphometric terms, include but are not limited to, age, gender, smoking history, body mass index (BMI) and hip to waist ratio.

In some examples, the methods of the disclosure also contemplate the inclusion of the subject's gender as an biomarker in a biomarker panel described herein. Without wishing to be bound by theory, the subject's gender can be factored into the logistic regression algorithm by assigning an arbitrary value for females and a different arbitrary value for males. As would be understood by the person skilled in the art, the numerical value of the arbitrary value is not important, however it is important that different arbitrary values are assigned for males and females. In one example, the subject's gender can be factored into the logistic regression algorithm by assigning an arbitrary value of 1 for females and 0 for males. In one example, the subject's gender can be factored into the logistic regression algorithm by assigning an arbitrary value of 1.1 for females and 1 for males.

In some examples, the methods of the disclosure also contemplate the inclusion of the subject's age as an biomarker in a biomarker panel described herein.

### Sample Preparation and Processing

Before analysing the biological sample, it may be desirable to perform one or more sample preparation operations upon the sample. Generally, these sample preparation operations may include such manipulations as extraction and isolation of intracellular material from a cell or tissue such as, the extraction of nucleic acids, protein, or other macromolecules from the samples.

Sample preparation which can be used with the methods of disclosure include but are not limited to, centrifugation, affinity chromatography, magnetic separation, fractionation, precipitation, and combinations thereof.

Sample preparation can further include dilution by an appropriate solvent and amount to ensure the appropriate range of concentration level is detected by a given assay.

Accessing the nucleic acids and macromolecules from the intercellular space of the sample may generally be performed by either physical, chemical methods, or a combination of both. In some applications of the methods, following the isolation of the crude extract, it will often be desirable to separate the nucleic acids, proteins, cell membrane particles, and the like. In some examples of the methods it will be desirable to keep the nucleic acids with its proteins, and cell membrane particles.

In some examples of the methods provided herein, nucleic acids and proteins can be extracted from a biological sample prior to analysis using methods of the disclosure. Extraction can be by means including, but not limited to, the use of detergent lysates, sonication, or vortexing with glass bead.

In some examples, molecules can be isolated using any technique suitable in the art including, but not limited to, techniques using gradient centrifugation (e.g., cesium chloride gradients, sucrose gradients, glucose gradients, etc.), centrifugation protocols, boiling, purification kits, and the use of liquid extraction with agent extraction methods such as methods using Trizol or DNAzol.

Samples may be prepared according to standard biological sample preparation depending on the desired detection method. For example for mass spectrometry detection, biological samples obtained from a patient may be centrifuged, filtered, processed by immunoaffinity column, separated into fractions, partially digested, and combinations thereof. Various fractions may be resuspended in appropriate carrier such as buffer or other type of loading solution for detection and analysis, including LCMS loading buffer.

### Biomarker Measurement

Measurement of a biomarker panel relates to a quantitative measurement of a plurality of biomarkers. The present disclosure provides for methods for detecting biomarkers in biological samples. Biomarkers can include but are not limited to proteins, DNA molecules, and RNA molecules. More specifically the present disclosure is based on the discovery of protein biomarkers that are differentially expressed in subjects that have an increased risk of acquiring colorectal cancer or have colorectal cancer. Therefore the detection of one or more of these differentially expressed biomarkers in a biological sample provides useful information whether or not a subject is at risk or suffering from colorectal cancer and what type of nature or state of the condition. Any suitable method known to the skilled person can be used to detect one or more of the biomarker described herein.

Useful analyte capture agents that can be used with the present disclosure include but are not limited to antibodies, such as crude serum containing antibodies, purified antibodies, monoclonal antibodies, polyclonal antibodies, synthetic antibodies, antibody fragments (for example, Fab fragments); antibody interacting agents, such as protein A, carbohydrate binding proteins, and other interactants; protein interactants (for example avidin and its derivatives); peptides; and small chemical entities, such as enzyme substrates, cofactors, metal ions/chelates, and haptens. Antibodies may be modified or chemically treated to optimize binding to targets or solid surfaces (e.g. biochips and columns).

In one particular example of the disclosure, the biomarker can be detected in a biological sample using an immunoassay. Immunoassays are assay that use an antibody that specifically bind to or recognizes an antigen (e.g. site on a protein or peptide, biomarker target). The method includes the steps of contacting the biological sample with the antibody and allowing the antibody to form a complex of with the antigen in the sample, washing the sample and detecting the antibody-antigen complex with a detection reagent. In one example, antibodies that recognize the biomarkers may be commercially available. In another examples, an antibody that recognizes the biomarkers may be generated by known methods of antibody production.

Alternatively, the marker in the sample can be detected using an indirect assay, wherein, for example, a second, labelled antibody is used to detect bound marker-specific antibody. Exemplary detectable labels include magnetic beads (e.g., DYNABEADS™), fluorescent dyes, radiolabels, enzymes (e.g., horse radish peroxide, alkaline phosphatase and others commonly used), and colorimetric labels such as colloidal gold or coloured glass or plastic beads. The marker in the sample can be detected using and/or in a competition or inhibition assay wherein, for example, a monoclonal antibody which binds to a distinct epitope of the marker is incubated simultaneously with the mixture.

The conditions to detect an antigen using an immunoassay will be dependent on the particular antibody used. Also, the incubation time will depend upon the assay format, marker, volume of solution, concentrations and the like. In general, the immunoassays will be carried out at room temperature, although they can be conducted over a range of temperatures, such as 10 degrees to 40 degrees Celsius depending on the antibody used.

There are various types of immunoassay known in the art that, as a starting basis, can be used to tailor the assay for the detection of the biomarkers of the present disclosure. Useful assays can include, for example, an enzyme immune assay (EIA) such as enzyme-linked immunosorbent assay (ELISA), including the sandwich ELISA. There are many variants of these approaches, but those are based on a similar idea. For example, if an antigen can be bound to a solid support or surface, it can be detected by reacting it with a specific antibody and the antibody can be quantitated by reacting it with either a secondary antibody or by incorporating a label directly into the primary antibody. Alternatively, an antibody can be bound to a solid surface and the antigen added. A second antibody that recognizes a distinct epitope on the antigen can then be added and detected. This is frequently called a ‘sandwich assay’ and can frequently be used to avoid problems of high background or non-specific reactions. These types of assays are sensitive and reproducible enough to measure low concentrations of antigens in a biological sample.

Immunoassays can be used to determine presence or absence of a marker in a sample as well as the quantity of a marker in a sample. Methods for measuring the amount of, or presence of, antibody-marker complex include but are not limited to, fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). In general these regents are used with optical detection methods, such as various forms of microscopy, imaging methods and non-imaging methods. Electrochemical methods include voltammetry, amperometry and electrochemiluminescence methods. Radio frequency methods include multipolar resonance spectroscopy.

In one example, the disclosure can use antibodies for the detection of the biomarkers. Antibodies can be made that specifically bind to the biomarkers of the present assay can be prepared using standard methods known in the art. For example polyclonal antibodies can be produced by injecting an antigen into a mammal, such as a mouse, rat, rabbit, goat, sheep, or horse for large quantities of antibody. Blood isolated from these animals contains polyclonal antibodies—multiple antibodies that bind to the same antigen. Alternatively polyclonal antibodies can be produced by injecting the antigen into chickens for generation of polyclonal antibodies in egg yolk. In addition, antibodies can be made that specifically recognize modified forms for the biomarkers such as a phosphorylated form of the biomarker, that is to say, they will recognize a tyrosine or a serine after phosphorylation, but not in the absence of phosphate. In this way antibodies can be used to determine the phosphorylation state of a particular biomarker.

Antibodies can be obtained commercially or produced using well-established methods. To obtain antibody that is specific for a single epitope of an antigen, antibody-secreting lymphocytes are isolated from the animal and immortalized by fusing them with a cancer cell line. The fused cells are called hybridomas, and will continually grow and secrete antibody in culture. Single hybridoma cells are isolated by dilution cloning to generate cell clones that all produce the same antibody; these antibodies are called monoclonal antibodies.

Polyclonal and monoclonal antibodies can be purified in several ways. For example, one can isolate an antibody using antigen-affinity chromatography which is couple to bacterial proteins such as Protein A, Protein G, Protein L or the recombinant fusion protein, Protein A/G followed by detection of via UV light at 280 nm absorbance of the eluate fractions to determine which fractions contain the antibody. Protein A/G binds to all subclasses of human IgG, making it useful for purifying polyclonal or monoclonal IgG antibodies whose subclasses have not been determined. In addition, it binds to IgA, IgE, IgM and (to a lesser extent) IgD. Protein A/G also binds to all subclasses of mouse IgG but does not bind mouse IgA, IgM or serum albumin. This feature, allows Protein A/G to be used for purification and detection of mouse monoclonal IgG antibodies, without interference from IgA, IgM and serum albumin.

Antibodies can be derived from different classes or isotypes of molecules such as, for example, IgA, IgA IgD, IgE, IgM and IgG. The antibody that is most useful in biological studies is the IgG class, a protein molecule that is made and secreted and can recognize specific antigens. The IgG is composed of two subunits including two “heavy” chains and two “light” chains. These are assembled in a symmetrical structure and each IgG has two identical antigen recognition domains. The antigen recognition domain is a combination of amino acids from both the heavy and light chains. The molecule is roughly shaped like a “Y” and the arms/tips of the molecule comprise the antigen-recognizing regions or Fab (fragment, antigen binding) region, while the stem of Fc (Fragment, crystallizable) region is not involved in recognition and is fairly constant. The constant region is identical in all antibodies of the same isotype, but differs in antibodies of different isotypes.

It is also possible to use an antibody to detect a protein after fractionation by western blotting. In one example, the disclosure can use western blotting for the detection of the biomarkers. Western blot (protein immunoblot) is an analytical technique used to detect specific proteins in the given sample or protein extract from a sample. It uses gel electrophoresis, SDS-PAGE to separate either native proteins by their 3-dimensional structure or it can be run under denaturing conditions to separate proteins by their length. After separation by gel electrophoresis, the proteins are then transferred to a membrane (typically nitrocellulose or PVDF). The proteins transferred from the SDS-PAGE to a membrane can then be incubated with particular antibodies under gentle agitation, rinsed to remove non-specific binding and the protein-antibody complex bound to the blot can be detected using either a one-step or two step detection methods. The one step method includes a probe antibody which both recognizes the protein of interest and contains a detectable label, probes which are often available for known protein tags. The two-step detection method involves a secondary antibody that has a reporter enzyme or reporter bound to it. With appropriate reference controls, this approach can be used to measure the abundance of a protein.

In one example, the method of the disclosure can use flow cytometry. Flow cytometry is a laser based, biophysical technology that can be used for biomarker detection, quantification (cell counting) and cell isolation. This technology is routinely used in the diagnosis of health disorders, especially blood cancers. In general, flow cytometry works by suspending single cells in a stream of fluid, a beam of light (usually laser light) of a single wavelength is directed onto the stream of liquid, and the scatter light caused by the passing cell is detected by an electronic detection apparatus. Fluorescence-activated cell sorting (FACS) is a specialized type of flow cytometry that often uses the aid of florescent-labelled antibodies to detect antigens on cell of interest. This additional feature of antibody labelling use in FACS provides for simultaneous multiparametric analysis and quantification based upon the specific light scattering and fluorescent characteristics of each cell florescent-labelled cell and it provides physical separation of the population of cells of interest as well as traditional flow cytometry does.

In another example, the flow cytometry is combined with bead systems, wherein the target antigen is attached to a bead. Such systems are known to persons skilled in the art.

A wide range of fluorophores can be used as labels in flow cytometry. Fluorophores are typically attached to an antibody that recognizes a target feature on or in the cell. Examples of suitable fluorescent labels include, but are not limited to: fluorescein (FITC), 5,6-carboxymethyl fluorescein, Texas red, nitrobenz-2-oxa-1,3-diazol-4-yl (NBD), and the cyanine dyes Cy3, Cy3.5, Cy5, Cy5.5 and Cy7. Other Fluorescent labels such as Alexa Fluor® dyes, DNA content dye such as DAPI, Hoechst dyes are well known in the art and all can be easily obtained from a variety of commercial sources. Each fluorophore has a characteristic peak excitation and emission wavelength, and the emission spectra often overlap. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus choosing one that do not have a lot of spectra overlap allows their simultaneous detection. The fluorescent labels can be obtained from a variety of commercial sources. The maximum number of distinguishable fluorescent labels is thought to be around approximately 17 or 18 different fluorescent labels. This level of complex read-out necessitates laborious optimization to limit artefacts, as well as complex deconvolution algorithms to separate overlapping spectra. Quantum dots are sometimes used in place of traditional fluorophores because of their narrower emission peaks. Other methods that can be used for detecting include isotope labelled antibodies, such as lanthanide isotopes. However this technology ultimately destroys the cells, precluding their recovery for further analysis.

In one example, the method of the disclosure can use immunohistochemistry for detecting the expression levels of the biomarkers of the present disclosure. Thus, antibodies specific for each marker are used to detect expression of the claimed biomarkers in a biological sample. The antibodies can be detected by direct labelling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labelled secondary antibody, comprising antisera, polyclonal antisera or a monoclonal antibody specific for the primary antibody. Immunohistochemistry protocols are well known in the art and protocols and antibodies are commercially available. Alternatively, one could make an antibody to the biomarkers or modified versions of the biomarker or binding partners as disclosure herein that would be useful for determining the expression levels of in a biological sample.

In one example, the method of the disclosure can use a biochip. Biochips can be used to screen a large number of macromolecules. In this technology macromolecules are attached to the surface of the biochip in an ordered array format. The grid pattern of the test regions allowed analysed by imaging software to rapidly and simultaneously quantify the individual analytes at their predetermined locations (addresses). The CCD camera is a sensitive and high-resolution sensor able to accurately detect and quantify very low levels of light on the chip.

Biochips can be designed with immobilized nucleic acid molecules, full-length proteins, antibodies, affibodies (small molecules engineered to mimic monoclonal antibodies), aptamers (nucleic acid-based ligands) or chemical compounds. A chip could be designed to detect multiple macromolecule types on one chip. For example, a chip could be designed to detect nucleic acid molecules, proteins and metabolites on one chip. The biochip is used and designed to simultaneously analyze a panel biomarker in a single sample, producing a subjects profile for these biomarkers. The use of the biochip allows for the multiple analyses to be performed reducing the overall processing time and the amount of sample required.

Protein microarrays are a particular type of biochip which can be used with the present disclosure. The chip consists of a support surface such as a glass slide, nitrocellulose membrane, bead, or microtitre plate, to which an array of capture proteins are bound in an arrayed format onto a solid surface. Protein array detection methods must give a high signal and a low background. Detection probe molecules, typically labelled with a fluorescent dye, are added to the array. Any reaction between the probe and the immobilized protein emits a fluorescent signal that is read by a laser scanner. Such protein microarrays are rapid, can be automated, and offer high sensitivity of protein biomarker read-outs for diagnostic tests. However, it would be immediately appreciated to those skilled in the art that there is a variety of detection methods that can be used with this technology.

There are at least three types of protein microarrays that are currently used to study the biochemical activities of proteins. For example there are analytical microarrays (also known as capture arrays), Functional protein microarrays (also known as target protein arrays) and Reverse phase protein microarrays (RPA).

The present disclosure provides for the detection of the biomarkers using an analytical protein microarray, such as Luminex xMAP Technology. Analytical protein microarrays are constructed using a library of antibodies, aptamers or affibodies. The array is probed with a complex protein solution such as a blood, serum or a cell lysate that function by capturing protein molecules they specifically bind to. Analysis of the resulting binding reactions using various detection systems can provide information about expression levels of particular proteins in the sample as well as measurements of binding affinities and specificities. This type of protein microarray is especially useful in comparing protein expression in different samples.

In one example, the method of the disclosure can use functional protein microarrays. These are constructed by immobilising large numbers of purified full-length functional proteins or protein domains and are used to identify protein-protein, protein-DNA, protein-RNA, protein-phospholipid, and protein-small molecule interactions, to assay enzymatic activity and to detect antibodies and demonstrate their specificity. These protein microarray biochips can be used to study the biochemical activities of the entire proteome in a sample.

In one example, the method of the disclosure can use reverse phase protein microarrays (RPA). Reverse phase protein microarrays are constructed from tissue and cell lysates that are arrayed onto the microarray and probed with antibodies against the target protein of interest. These antibodies are typically detected with chemiluminescent, fluorescent or colorimetric assays. In addition to the protein in the lysate, reference control peptides are printed on the slides to allow for protein quantification. RPAs allow for the determination of the presence of altered proteins or other agents that may be the result of disease and present in a diseased cell.

In some examples detection of biomarkers utilises the ARCHITECT system (Abbott).

The present disclosure provides for the detection of the biomarkers using mass spectroscopy (alternatively referred to as mass spectrometry). Mass spectrometry (MS) is an analytical technique that measures the mass-to-charge ratio of charged particles. It is primarily used for determining the elemental composition of a sample or molecules, and for elucidating the chemical structures of molecules, such as peptides and other chemical compounds. MS works by ionizing chemical compounds to generate charged molecules or molecule fragments and measuring their mass-to-charge ratios. MS instruments typically consist of three modules (1) an ion source, which can convert gas phase sample molecules into ions (or, in the case of electrospray ionization, move ions that exist in solution into the gas phase) (2) a mass analyzer, which sorts the ions by their masses by applying electromagnetic fields and (3) detector, which measures the value of an indicator quantity and thus provides data for calculating the abundances of each ion present.

Suitable mass spectrometry methods to be used with the present disclosure include but are not limited to, one or more of electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), tandem liquid chromatography-mass spectrometry (LC-MS/MS) mass spectrometry, desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS), atmospheric pressure photoionization mass spectrometry (APPI-MS), APPI-MS/MS, and APPI-(MS)n, quadrupole mass spectrometry, Fourier transform mass spectrometry (FTMS), and ion trap mass spectrometry, where n is an integer greater than zero.

To gain insight into the underlying proteomics of a sample, LC-MS is commonly used to resolve the components of a complex mixture. The LC-MS method generally involves protease digestion and denaturation (usually involving a protease, such as trypsin and a denaturant such as, urea to denature tertiary structure and iodoacetamide to cap cysteine residues) followed by LC-MS with peptide mass fingerprinting or LC-MS/MS (tandem MS) to derive sequence of individual peptides. LC-MS/MS is most commonly used for proteomic analysis of complex samples where peptide masses may overlap even with a high-resolution mass spectrometer. Samples of complex biological fluids like human serum may be first separated on an SDS-PAGE gel or HPLC-SCX and then run in LC-MS/MS allowing for the identification of over 1000 proteins.

While multiple mass spectrometric approaches can be used with the methods of the disclosure as provided herein, in some applications it may be desired to quantify proteins in biological samples from a selected subset of proteins of interest. One such MS technique that can be used with the present disclosure is Multiple Reaction Monitoring Mass Spectrometry (MRM-MS), or alternatively referred to as Selected Reaction Monitoring Mass Spectrometry (SRM-MS).

The MRM-MS technique uses a triple quadrupole (QQQ) mass spectrometer to select a positively charged ion from the peptide of interest, fragment the positively charged ion and then measure the abundance of a selected positively charged fragment ion. This measurement is commonly referred to as a transition.

In some applications the MRM-MS is coupled with High-Pressure Liquid Chromatography (HPLC) and more recently Ultra High-Pressure Liquid Chromatography (UHPLC). In other applications MRM-MS is coupled with UHPLC with a QQQ mass spectrometer to make the desired LC-MS transition measurements for all of the peptides and proteins of interest.

In some applications the utilization of a quadrupole time-of-flight (qTOF) mass spectrometer, time-of-flight (TOF-TOF) mass spectrometer, Orbitrap mass spectrometer, quadrupole Orbitrap mass spectrometer or any Quadrupolar Ion Trap mass spectrometer can be used to select for a positively charged ion from one or more proteins of interest. The fragmented, positively charged ions can then be measured to determine the abundance of a positively charged ion for the quantitation of the peptide or protein of interest.

In some applications the utilization of a time-of-flight (TOF), quadrupole time-of-flight (qTOF) mass spectrometer, time-of-flight (TOF-TOF) mass spectrometer, Orbitrap mass spectrometer or quadrupole Orbitrap mass spectrometer can be used to measure the mass and abundance of a positively charged peptide ion from the protein of interest without fragmentation for quantitation. In this application, the accuracy of the analyte mass measurement can be used as selection criteria of the assay. An isotopically labelled internal standard of a known composition and concentration can be used as part of the mass spectrometric quantitation methodology.

In some applications, time-of-flight (TOF), quadrupole time-of-flight (qTOF) mass spectrometer, time-of-flight (TOF-TOF) mass spectrometer, Orbitrap mass spectrometer or quadrupole Orbitrap mass spectrometer can be used to measure the mass and abundance of a protein of interest for quantitation. In this application, the accuracy of the analyte mass measurement can be used as selection criteria of the assay. Optionally this application can use proteolytic digestion of the protein prior to analysis by mass spectrometry. An isotopically labelled internal standard of a known composition and concentration can be used as part of the mass spectrometric quantitation methodology.

In some applications, various ionization techniques can be coupled to the mass spectrometers provided herein to generate the desired information. Non-limiting exemplary ionization techniques that can be used with the present disclosure include but are not limited to Matrix Assisted Laser Desorption Ionization (MALDI), Desorption Electrospray Ionization (DESI), Direct Assisted Real Time (DART), Surface Assisted Laser Desorption Ionization (SALDI), or Electrospray Ionization (ESI).

In some applications, HPLC and UHPLC can be coupled to a mass spectrometer a number of other protein separation techniques can be performed prior to mass spectrometric analysis. Some exemplary separation techniques which can be used for separation of the desired analyte (e.g., peptide or protein) from the matrix background include but are not limited to Reverse Phase Liquid Chromatography (RP-LC) of proteins or peptides, offline Liquid Chromatography (LC) prior to MALDI, 1 dimensional gel separation, 2-dimensional gel separation, Strong Cation Exchange (SCX) chromatography, Strong Anion Exchange (SAX) chromatography, Weak Cation Exchange (WCX), and Weak Anion Exchange (WAX). One or more of the above techniques can be used prior to mass spectrometric analysis.

In one example of the disclosure the biomarker can be detected in a biological sample using a microarray. Differential gene expression can also be identified, or confirmed using the microarray technique. Thus, the expression profile biomarkers can be measured in either fresh or fixed tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. The source of mRNA typically is total RNA isolated from a biological sample, and corresponding normal tissues or cell lines may be used to determine differential expression.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labelled cDNA probes may be generated through incorporation of fluorescent nucleotides by reverse transcription of RNA extracted from tissues of interest. Labelled cDNA probes applied to the chip hybridize with specificity to each spot of DNA on the array. After stringent washing to remove non-specifically bound probes, the microarray chip is scanned by a device such as, confocal laser microscopy or by another detection method, such as a CCD camera. Quantitation of hybridization of each arrayed element allows for assessment of corresponding mRNA abundance. With dual colour fluorescence, separately labelled cDNA probes generated from two sources of RNA are hybridized pair-wise to the array. The relative abundance of the transcripts from the two sources corresponding to each specified gene is thus determined simultaneously. Microarray analysis can be performed by commercially available equipment, following manufacturer's protocols.

In one example of the disclosure, the biomarker can be detected in a biological sample using qRT-PCR, which can be used to compare mRNA levels in different sample populations, in normal and tumor tissues, with or without drug treatment, to characterize patterns of gene expression, to discriminate between closely related mRNAs, and to analyse RNA structure. The first step in gene expression profiling by RT-PCR is extracting RNA from a biological sample followed by the reverse transcription of the RNA template into cDNA and amplification by a PCR reaction. The reverse transcription reaction step is generally primed using specific primers, random hexamers, or oligo-dT primers, depending on the goal of expression profiling. The two commonly used reverse transcriptases are avilo myeloblastosis virus reverse transcriptase (AMV-RT) and Moloney murine leukemia virus reverse transcriptase (MLV-RT).

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan™ PCR typically utilizes the 5′-nuclease activity of Taq or Tth polymerase to hydrolyse a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labelled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan™ RT-PCR can be performed using commercially available equipment, such as, for example, ABI PRISM 7700 Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system includes software for running the instrument and for analysing the data. 5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and Beta-Actin.

A more recent variation of the RT-PCR technique is the real time quantitative PCR, which measures PCR product accumulation through a dual-labelled fluorigenic probe (i.e., TaqMan™ probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996). Other quantitative methods include digital droplet PCR.

Typically, quantification of biomarkers as performed in the present disclosure will include referenced control samples. In some examples, the control reference is determined from measurements of the biomarkers in corresponding panel of biomarkers from a population of healthy individuals. The term “healthy individual” as used herein refers to a person or populations of persons who are known not to have colorectal cancer, such knowledge being derived from clinical data on the individual which may have been determined from colonoscopy or sigmoidoscopy. In some examples, the control reference is determined from measurements of the corresponding biomarkers in a “typical population”. Preferably, a “typical population” will exhibit a spectrum of colorectal cancer at different stages of disease progression. It is particularly preferred that a “typical population” exhibits the expression characteristics of a cohort of subjects as described herein.

In another example, the control reference may be derived from an established data set including one or more of:
1. a data set comprising measurements of the biomarkers for a population of subjects known to have colorectal cancer;
2. a data set comprising measurements of biomarkers for the subject being tested wherein said measurements have been made previously, such as, for example, when the subject was known to be healthy or, in the case of a subject having colorectal cancer, when the subject was diagnosed or at an earlier stage in disease progression; and/or
3. a data set comprising measurements of the biomarkers for a healthy individual or a population of healthy individuals.

### Data Analysis

In some examples, methods of determining whether a subject has colorectal cancer or is otherwise at an increased risk of developing colorectal cancer are based upon the biomarker panel measurement compared to a reference profile that can be made in conjunction with statistical analysis.

A quantitative score may be determined by the application of a specific algorithm. The algorithm used to calculate the quantitative score in the methods disclosed herein may group the expression level values of a biomarker or groups of biomarkers. The formation of a particular group of biomarkers, in addition, can facilitate the mathematical weighting of the contribution of various expression levels of biomarker or biomarker subsets (e.g. classifier) to the quantitative score.

In some examples, SPSS software may be used for the statistical analysis. In some examples, binary logistic regression analysis may be used to predict he diagnostic efficiency of the selected biomarkers. In some examples, a statistical algorithm used with a computer to implement the statistical algorithm may be used. In some examples, the statistical algorithm is a learning statistical classifier system. Examples of such systems include Random Forest, interactive tree, classification and regression tree classification or neural networks.

A fair evaluation of a test requires its assessment using “out-of-sample” subjects, that is, subjects not included in the construction of the initial predictive model. This is achieved by assessing the test performance using n-fold cross validation.

Tests for statistical significance include linear and non-linear regression, including ANOVA, Kruskal-Wallis, Wilcoxon, Mann-Whitney and odds ratio, Bayesian probability algorithms. As the number of biomarkers measured increases however, it can be generally more convenient to use a more sophisticated technique such as Random Forests, simple logistic, or Bayes Net to name a few.

In some examples, Bayesian probability may be adopted. In this circumstance a 10-fold cross-validation can be used to estimate the “out-of-sample” performance of the models in question. For each combination of biomarkers under consideration, the data can be divided randomly into 10 sub-samples, each with similar proportions of healthy subject and subjects at each stage of disease. In turn, each subsample can be excluded, and a logistic model built using the remaining 90% of the subjects. This model can then be used to estimate the probability of cancer for the excluded sub-sample, providing an estimate of “out-of-sample” performance. By repeating this for the remaining 9 subsamples, “out-of-sample” performance can be estimated from the study data itself. These “out-of sample” predicted probabilities can then be compared with the actual disease status of the subjects to create a Receiver Operating Characteristic (ROC) Curve, from which the cross-validated sensitivity at a given specificity (e.g. 95% specificity) may be estimated.

Each estimate of “out-of-sample” performance using cross-validation (or any other method), whilst unbiased, has an element of variability to it. Hence a ranking of models (based on biomarker combinations) can be indicative only of the relative performance of such models. However a set of biomarkers which is capable of being used in a large number of combinations to generate a diagnostic test as demonstrated via “out-of-sample” performance evaluations, almost certainly contains within itself combinations of biomarkers that will withstand repeated evaluation.

In one example, a biomarkers are measured using the following algorithm:

$$log ( \frac{p}{1 - p} ) = {β}_{0} + {β}_{B M 1} {C}_{B M 1} + {β}_{B M 2} {C}_{B M 2} + {β}_{B M 3} {C}_{B M 3} + {β}_{B M 4} {C}_{B M 4} + … … … … … . {β}_{B M i} {C}_{B M i}$$

wherein ρ represents the probability that a person has colorectal cancer. Each C_{BMi} is the logarithm of concentration of the i^{th} biomarker in the plasma (or serum) of one subject in the cohort being tested. Each beta (β_{BM}) is a coefficient applying to that biomarker in the concentration units in which it is measured—β₀ is an “offset” or “intercept”. This linear logistic model is common to all results presented herein, but is far from the only way in which a combination of biomarker concentrations may be modelled to predict the probability of cancer. As would be appreciated by the person skilled in the art, while the base-10 logarithm of the concentration of biomarker is exemplified herein other logarithms can also be used, for example base-2 logarithm.

Other non-linear or linear logistic algorithms that would be equally applicable include Random Forest, Linear Models for MicroArray data (LIMMA) and/or Significance Analyses of Microarray Data (SAM), Best First, Greedy Stepwise, Naive Bayes, Linear Forward Selection, Scatter Search, Linear Discriminant Analysis (LDA), Stepwise Logistic Regression, Receiver Operating Characteristic and Classification Trees (CT).

The skilled person will be familiar with determination of co-efficient values in regression algorithms.

The algorithms described herein can be used to derive a cancer likelihood score. In some examples, a quantitative score is derived which may indicate an increased likelihood of poor clinical outcome, good clinical outcome, high risk of CRC, or low risk of CRC. The score may then inform treatment management.

In some examples, the biomarker panel is able to detect CRC with a sensitivity and specificity comparable to, or better than FOBT. The skilled person will know that sensitivity refers to the proportion of actual positives in the diagnostic test which are correctly identified as having colorectal cancer. Specificity measures the proportion of negatives which are correctly identified as not having colorectal cancer.

In some examples, the biomarker panel has a sensitivity of at least 50%, 60% or 65%, or at least 70%, 80%, 83%, 85%, 86%, 87%, 88%, 89%, 90%, 93% or at least 95%.

In some examples, the biomarker panel has a specificity of at least 75%, 80%, 85%, 90%, 91%, 92%, 93%, 94% or at least 95%.

### Data Handling

It will be apparent from the discussion herein that knowledge-based computer software and hardware for implementing an algorithm also form part of the present disclosure. Such computer software and/or hardware are useful for performing a method of detecting colorectal cancer according the disclosure.

The values from the assays described herein can be calculated and stored manually. Alternatively, the statistical analysis steps can be completely or partially performed by a computer program product. The present disclosure thus provides a computer program product including a computer readable storage medium having a computer program stored on it. The program can, when read by a computer, execute relevant calculations based on values obtained from analysis of one or more biological samples from a subject (e.g., gene or protein expression levels, normalization, standardization, thresholding, and conversion of values from assays to a clinical outcome score and/or text or graphical depiction of clinical status or stage and related information). The computer program product has stored therein a computer program for performing the calculation.

The present disclosure also provides systems for executing the data collection and handling or calculating software programs described above, which system generally includes: a) a central computing environment; b) an input device, operatively connected to the computing environment, to receive patient data, wherein the patient data can include, for example, gene or protein expression level or other value obtained from an assay using a biological sample from the subject, or mass spec data or data for any of the assays provided by the present disclosure; c) an output device, connected to the computing environment, to provide information to a user (e.g., medical personnel); and d) an algorithm executed by the central computing environment (e.g., a processor), where the algorithm is executed based on the data received by the input device, and wherein the algorithm calculates an expression score, thresholding, or other functions described herein. The methods provided by the present disclosure may also be automated in whole or in part. In some examples, the methods comprise a combination of laboratory based methods and computer based methods.

In one example, a method of the disclosure may be used in existing knowledge-based architecture or platforms associated with pathology services. For example, results from a method described herein are transmitted via a communications network (e.g. the internet) to a processing system in which an algorithm is stored and used to generate a predicted posterior probability value which translates to the score of disease probability which is then forwarded to an end user in the form of a diagnostic or predictive report.

The method of the disclosure may, therefore, be in the form of a kit or computer-based system which comprises the reagents necessary to detect the concentration of the biomarkers and the computer hardware and/or software to facilitate determination and transmission of reports to a clinician.

The assays described herein can be integrated into existing or newly developed pathology architecture or platform systems. For example, the present disclosure contemplates a method of allowing a user to determine a subject's risk with respect to colorectal cancer, the method including:
(a) receiving subject data obtained from determining a measurement of each biomarker in a biomarker panel described herein;
(b) processing the data via multivariate analysis (for example, regression analysis) to provide a disease score;
(c) determining the status of the subject in accordance with the results of the disease score in comparison with predetermined values; and
(d) transferring an indication of the status of the subject to the user via the communications network reference to the multivariate analysis which includes an algorithm which performs the multivariate analysis function.

### Kits

The present invention provides kits for the detection of biomarkers. Such kits may be suitable for detection of nucleic acid species, or alternatively may be for detection of a protein or polypeptide.

For detection of polypeptides, antibodies will most typically be used as components of kits. However, any agent capable of binding specifically to a biomarker gene product will be useful. Other components of the kits will typically include labels, secondary antibodies, inhibitors, co-factors and control gene or protein product preparations to allow the user to quantitate expression levels and/or to assess whether the measurement has worked correctly. Enzyme-linked immunosorbent assay-based (ELISA) tests and competitive ELISA tests are particularly suitable assays that can be carried out easily by the skilled person using kit components.

In some examples, the kit may comprise a microtitre plate on which is immobilised capture antibodies corresponding to the biomarkers being measured.

In some examples, the kit comprises beads on which is immobilised capture antibodies corresponding to the biomarkers being measured.

Optionally, the kit further comprises means for the detection of the binding of an antibody to a biomarker polypeptide. Such means include a reporter molecule such as, for example, an enzyme (such as horseradish peroxidase or alkaline phosphatase), a dye, a radionucleotide, a luminescent group, a chemiluminescent group, a fluorescent group, biotin or a colloidal particle, such as colloidal gold or selenium. Preferably such a reporter molecule is directly linked to the antibody.

In one example, a kit may additionally comprise a reference sample. In one embodiment, a reference sample comprises a polypeptide that is detected by an antibody. Preferably, the polypeptide is of known concentration. Such a polypeptide is of particular use as a standard. Accordingly, various known concentrations of such a polypeptide may be detected using a diagnostic assay described herein.

For detection of nucleic acids, such kits may contain a first container such as a vial or plastic tube or a microtiter plate that contains an oligonucleotide probe. The kits may optionally contain a second container that holds primers. The probe may be hybridisable to DNA whose altered expression is associated with colorectal cancer and the primers are useful for amplifying this DNA. Kits that contain an oligonucleotide probe immobilised on a solid support could also be developed, for example, using arrays (see supplement of issue 21(1) Nature Genetics, 1999).

For PCR amplification of nucleic acid, nucleic acid primers may be included in the kit that are complementary to at least a portion of a biomarker gene as described herein. The set of primers typically includes at least two oligonucleotides, preferably four oligonucleotides, that are capable of specific amplification of DNA. Fluorescent-labelled oligonucleotides that will allow quantitative PCR determination may be included (e.g. TaqMan chemistry, Molecular Beacons). Suitable enzymes for amplification of the DNA, will also be included.

Control nucleic acid may be included for purposes of comparison or validation. Such controls could either be RNA/DNA isolated from healthy tissue, or from healthy individuals, or housekeeping genes such as β-actin or GAPDH whose mRNA levels are not affected by colorectal cancer.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

### Material and Methods

### Ethics

All research protocols used in this study were approved by the relevant Human Research Ethics Committees. Written informed consent was obtained from each participant prior to blood sample collection.

### Participant Samples

A collection of plasma and serum samples was taken and processed from a cohort of ninety-six colorectal cancer patients (Dukes Stages I-IV) that were being treated at several hospitals. The samples were obtained from the Victorian Cancer Biobank.

Blood was collected and processed from a group of about 50 healthy volunteers (controls) over the age of 50 and between the ages of 50-85.

Subjects who had already received chemotherapy and/or radiotherapy were excluded from the analysis. The characteristics of the subjects are summarised in Table 1 below.

TABLE 1
Patient characteristics (study 3)

| Characteristics | Control | Colorectal cancer |
|---|---|---|
| N | 50 | 95 |

Gender N

| | | |
|---|---|---|
| Female | 25 | 50 |
| Male | 25 | 45 |
| Median age, yrs (range) | 70 (50-85) | 67 (44-93) |

Cancer Stage

| | |
|---|---|
| I | 21 |
| II | 31 |
| III | 33 |
| IV | 10 |

### Blood Collection and Processing

Serum samples from subjects were collected using a standard operating procedure as previously described (Brierley G V, et al. (2013) Cancer Biomark. 13: 67-73). Blood was collected into serum gel tubes (Scientific Specialties Inc., USA) and each sample was left to stand at room temperature for at least 30 min prior to centrifugation (1,200 g, 10 min, room temperature). The serum fraction was then transferred to clean 15 mL tubes and centrifuged again (1,800 g, 10 min, room temperature) prior to being aliquoted (250 μL) and stored (−80° C.). All samples were processed and stored within 2 hrs of collection. Serum samples were only thawed once prior to use.

### Blood Biomarkers and Biomarker Analysis

A total of 18 biomarkers were assessed. The markers assessed are summarised in Table 2 below.

TABLE 2
Biomarkers analysed

| | UniProt | NCBI |
|---|---|---|
| Biomarker | Reference | Accession No. |
| Brain derived neurotrophic factor (BDNF) | P23560 | NG_011794.1 |
| TIMP tissue inhibitor metallopeptidase 1 (TIMP1) | P01033 | NM_003254.2 |
| Matrix metallopeptidase 1 (MMP1) | P03956 | NG_011740.2 |
| Matrix metallopeptidase 7 (MMP7) | P09237 | NM_002423.5 |
| Insulin like growth factor 2 (IGF-II) | P01344 | NG_008849.1 |
| Soluble fraction of cytokeratin 18 protein (referred to | P05783 | NM_000224.3 |
| herein as M65) full length protein | | |
| Transforming growth factor beta 1 (TGFβ1, also | P01137 | NM_000660.4 |
| referred to as TGFBETA1, TGFb1) | | |
| Interleukin 8 (IL-8)/CXCL8 | P10145 | NM_000584.2 |
| Tumour necrosis factor alpha (TNFα, also referred to | P01375 | MH180383.1 |
| as TNFa, TNFalpha) | | |
| Mac-2-binding protein (MAC2BP) | Q08380 | NM_005567.3 |
| Caspase fragment cytokeratin 18 protein (referred to | P05783 | NM_000224.3 |
| herein as M30)/Apoptosense fragment containing | | |
| the neo epitope K18Asp396-NE. | | |
| Macrophage inflammatory protein (MIP1β, also | P13236 | NM_002984.2 |
| referred to herein as MIP1B, MIP1beta) | | |
| Interleukin 6 (IL-6) | P05231 | NG_011640.1 |
| Interleukin 13 (IL-13) | P35225 | NM_002188.2 |
| Lipocalin 2 (LCN2) | P80188 | NM_005564.5 |
| Insulin like growth factor binding protein 2 (IGFBP2) | P18065 | NM_000597.2 |
| Dickkopf-related protein 3 (DKK-3) | Q9UBP4 | NM_015881.5; |
| | | NM_013253; |
| | | NM_001018057.1 |
| Faecal Tumor M2-PK (Tumor M2-PK) (also referred | P14618 | NM_002654.3; |
| to as M2PK, PKM2, tumor PKM2), including dimeric | | NM_182470.1; |
| form. | | NM_182471. |

### Blood Biomarker Analysis

Sandwich ELISA analysis was used to quantify the levels of the biomarkers in serum samples from patients using standard protocols. Analysis of biomarkers was done with commercial kits and sourced antibodies. Details of the biomarkers assessed and the antibodies/ELISA kits used are shown in Table 3.

TABLE 3
Sources of antibodies used in the study

| Marker name and synonyms | Antibody Source |
|---|---|
| DKK-3 | Human Dkk-3 DuoSet ELISA Development System (R&D |
| | DY1118) Head quartered Minneapolis USA, sourced |
| | through In Vitro Technologies, Pty Ltd, Victoria, Australia) |
| IGFBP2 | Human IGFBP-2 ELISA (Demeditec DEE005) |
| IL-8 | Milliplex MAP Kit High Sensitivity Human Cytokine |
| | (multiplexing IL8 and IL13) |
| | (Millipore #HSCYTO-60SK) Sourced from Merck/Millipore |
| | through Thermo Fisher Scientific, Scoresby, Victoria, |
| | Australia |
| IL-13 | Milliplex MAP Kit High Sensitivity Human Cytokine |
| | (multiplexing IL8 and IL13) |
| | (Millipore #HSCYTO-60SK) Sourced from Merck/Millipore |
| | through Thermo Fisher Scientific, Scoresby, Victoria, |
| | Australia |
| M2PK | ScheBo Tumor M2-PK ELISA EDTA-Plasma Test |
| | (#08)(ScheBo Biotech AG, Giessen, Germany, sourced |
| | through Abacus dx (9 University Drive, Meadowbrook Qld |
| | 4131, Australia) |
| Mac2BP | Human Mac-2BP Platinum ELISA (BMS234) (Bender |
| | MedSystems GmbH, Austria) |
| TGFβ1 | Human TGF-β1 Quantikine ELISA (R&D DB100B) Head |
| | quartered Minneapolis USA, sourced through In Vitro |
| | Technologies, Pty Ltd, Victoria, Australia) |
| TIMP1 | Human TIMP-1 Quantikine ELISA (R&D DTM100) Head |
| | quartered Minneapolis USA, sourced through In Vitro |
| | Technologies, Pty Ltd, Victoria, Australia) |
| BDNF | Human BDNF Quantikine ELISA (R&D DBD00) (R&D |
| | Systems, Minneapolis USA) |
| MMP1 | R&D Fluorokine MAP human MMP Base kit (LMP000) (R&D |
| | Systems, Minneapolis USA) |
| MMP7 | R&D Fluorokine MAP human MMP Base kit (LMP000) (R&D |
| | Systems, Minneapolis USA) |
| IGF II | DSL Human IGF2 ELISA (DSL Inc., Webster, TX, USA), |
| Cytokeratin 18 protein (M65) | Peviva M65 ELISA Ref 10020 (VLVBio, Sweden) |
| Tumour necrosis factor alpha | Human TNF-alpha Quantikine ELISA Kit (MTA00B) (R&D |
| (TNFα) | Systems, Minneapolis USA) |
| Cytokeratin 18 protein (M30) | Peviva M30 Apoptosense ELISA Ref 10010 |
| | (VLVBio, Sweden) |
| Macrophage inflammatory | Flourokine MAP Human Base Kit A (R &D #LUH000 (R&D |
| protein (MIP1β) | Systems, Minneapolos USA) |
| Lipocalin 2 (LCN2) | Human Lipocalin-2 Quantikine ELISA (R&D DLCN20) R&D |
| | Systems, Minneapolis, USA) |
| Interleukin 6 (IL-6) | R&D Systems cytokine kit |

For each assay, samples were measured in duplicate and in-house quality control (QC) samples were included. QC samples consisted of pooled normal and pooled CRC patient serum samples.

For the standard ELISA, the absorbance or fluorescence signal was detected using the Wallac Victor3 1420 multilabel counter (Perkin Elmer, USA). Biomarker concentrations were derived from the respective standard curve using the WorkOut software (Qiagen, Hilden Germany).

### Sensitivity and Specificity Determination

The diagnostic potential of any given test is typically expressed in terms of its sensitivity and specificity for a given disease. The results for a given case/control experiment can be allocated into one of four quadrants illustrated in Table 4.

TABLE 4
Categories required to analyse results
in terms of sensitivity and specificity

| | Condition present (+) | Condition absent (−) |
|---|---|---|
| | by colonoscopy | by colonoscopy |
| Test positive | True Positive (TP) | False Positive (FP) |
| Test negative | False Negative (FN) | True Negative (TN) |
| | TP + FN | FP + TN |

Sensitivity is a measure of the test's ability to accurately detect those people with colorectal cancer using the diagnosis from colonoscopy and histopathology as the reference and is determined by the equation:

$$Test Sensitivity ( \% ) = 100 \% \times TP / ( TP + FN )$$

Specificity is the measure of the test's ability to accurately detect those people who do not have colorectal cancer using the colonoscopy/histopathology result as a reference and is determined by the equation:

$$Test Specificity ( \% ) = 100 \% \times TN / ( FP + TN )$$

A threshold concentration of a given biomarker (the ELISA-determined serum concentration of the biomarker protein) needs to be selected in order to define which patient results are considered to be positive or negative according to the ELISA test. It is possible to determine sensitivity and specificity of the test at any threshold concentration value across the entire range of concentrations observed in an experiment. There is an inverse relationship between sensitivity and specificity.

### Statistical Evaluation and Modelling

To find combinations of biomarkers that best separated controls and colorectal cancer patients, logistic regression based on the following equation was utilised as shown in the following equation:

$$Y i = {β}_{0} + {β}_{1} [ {M}_{1} ] + {β}_{2} [ {M}_{2} ] … + {ε}_{i}$$

### Where:

Yi is a binary indicator of presence or absence of CRC, as determined by colonoscopy in the experimental cohort.
β₀ is the regression intercept value.
M₁ etc. is the base-10 logarithm of the concentration of biomarker 1, as measured in specified units. M₂, M₃ etc. represents each individual biomarker.
β₁ etc. are the coefficients that are multiplied by the logged biomarker concentration.
εᵢ is an error term associated with the model.

To find combinations of biomarkers plus age that best separated controls and colorectal cancer patients, logistic regression based on the following equation was utilised as shown in the following equation:

$$Yi = {β}_{0} + β 1 [ {M}_{1} ] + {β}_{2} [ {M}_{2} ] … + {β}_{age} ( Age ) + {ε}_{i}$$

### Where:

Yi is a binary indicator of presence or absence of CRC, as determined by colonoscopy in the experimental cohort.
β₀ is the regression intercept value.
M₁ etc. is the base-10 logarithm of the concentration of biomarker 1, as measured in specified units. M₂, M₃ etc. represents each individual biomarker.
β₁ etc. are the coefficients that are multiplied by the logged biomarker concentration.
Age is the subjects age in years.
β_{age} is the coefficient that is multiplied by the subject's age in years.
εᵢ is an error term associated with the model.

Building models using different combinations of biomarkers and varying values for the relevant β₀₋₁₀ coefficients, those models most closely approximating the true value Yi can be determined for panels of biomarkers of different size.

To counter problems like overfitting or selection bias often encountered in statistical and machine learning processes, and to give insight into how any given model will generalise to an independent data set, data for each marker were reanalysed using 10-fold cross validation. Briefly, the full data set for any marker was divided into 10 equal sized sub-samples. One sub-sample was retained as a validation data set and the remaining 9 sub-samples were used as training data. This process was repeated 10 times with each of the sub-samples used exactly once as the validation data. The data presented in the tables below were obtained using this cross validation procedure.

Results for each assay were analysed using the statistical software packages Prism and “R”. Individual performance of markers was evaluated using the non-parametric Mann-Whitney t-test and individual receiver operator characteristic (ROC) curves were generated.

### Example 1 Analysis of Individual Biomarkers

The clinical characteristics for the subjects analysed in this study are shown in Table 1. A total of 95 subjects with confirmed diagnosis of colorectal cancer (CRC) by colonoscopy were analysed alongside 50 healthy controls. Of the CRC subjects, the median age was 67 years. The proportion of males to females was roughly equal.

Individual biomarkers were assessed by ELISA assay and the statistical difference between the medians for cases and controls for each biomarker was assessed using the Mann-Whitney Two Tailed T test. Biomarkers individually differentiating between colorectal cancer subjects and control subjects with p<0.05 were BDNF, TIMP1, TNFα, MAC2BP, MMP1, MMP7, IGFII, M65, TGFβ1, IL6, IL8, VEGFA, IGFBP2, DKK3 and M2PK.

Biomarkers M2PK, TIMP1, IGFBP2, BDNF, IL6 and IL8 appeared to provide the greatest discrimination between colorectal cancer subjects and controls according to Table 5.

Each biomarker (with and without age) was investigated for their ability to differentiate between colorectal cancer subjects and control subjects across all individuals and when the data was separated based on male/female. The cross-validated sensitivity for each individual biomarker (with and without age) is presented in Table 5. The cross-validated sensitivities were derived from application of logistic regression to the concentration values for each biomarker considered individually and represent the best sensitivity achievable at 95% specificity by differentially weighting the log concentration value for the particular biomarker and applied to all case and control samples.

TABLE 5
Cross validated sensitivity (as %) at 95% specificity for individual
biomarkers which were investigated for their ability to differentiate
between colorectal cancer subjects and control subjects.

| | Combined | Combined | Female | Female | Male | Male |
|---|---|---|---|---|---|---|
| | (including | (without | (including | (without | (including | (without |
| Biomarker | age) | age) | age) | age) | age) | age) |
| M2PK | 54.0 | 57.2 | 60.8 | 57.0 | 55.6 | 56.5 |
| IL6 | 29.0 | 25.8 | 32.9 | 25.3 | 19.6 | 17.8 |
| IL8 | 28.5 | 34.9 | 51.9 | 51.9 | 19.6 | 17.8 |
| IGFBP2 | 24.0 | 24.0 | 19.3 | 16.9 | 20.2 | 23.9 |
| TIMP1 | 23.0 | 25.7 | 10.8 | 19.3 | 22.2 | 20.4 |
| BDNF | 20.1 | 23.8 | 24.4 | 24.4 | 19.6 | 19.6 |
| LIPOCALIN | 19.1 | 20.2 | 23.7 | 25.0 | 16.7 | 17.6 |
| MMP7 | 16.4 | 14.3 | 9.9 | 7.4 | 13.0 | 14.8 |
| M65 | 14.9 | 17.6 | 20.0 | 18.8 | 13.0 | 15.7 |
| MMP1 | 11.0 | 11.5 | 12.2 | 11.0 | 11.0 | 8.3 |
| MIP1B | 9.9 | 5.2 | 9.5 | 0.0 | 11.1 | 15.7 |
| IGFII | 9.5 | 5.8 | 30.9 | 16.0 | 13.8 | 11.0 |
| DKK3 | 8.9 | 16.1 | 7.2 | 10.8 | 21.1 | 22.0 |
| TGFβ | 8.4 | 7.9 | 28.9 | 9.6 | 11.2 | 5.6 |
| IL13 | 6.3 | 4.5 | 1.2 | 1.7 | 12.0 | 6.6 |
| MAC2BP | 4.8 | 4.2 | 14.6 | 19.5 | 4.7 | 6.5 |
| TNFα | 4.3 | 5.9 | 0.0 | 12.7 | 1.9 | 5.6 |
| M30 | 2.1 | 11.0 | 4.9 | 11.0 | 4.6 | 6.4 |

Considered individually, biomarkers of greatest diagnostic potential couple high sensitivities for the detection of a given disease with high specificities. No individual biomarker, either alone or in combination with age, differentiated between cancer and normal controls with sufficient sensitivity at 95% specificity to be useful as a stand-alone biomarker for screening applications.

### Example 2 Analysis of Biomarker Combinations

In order to identify combinations of biomarkers that provided the best performance for colorectal cancer detection, the inventors measured the level of the eighteen lead biomarkers (as identified in Table 3) in serum samples from a new cohort of subjects with properties described in Table 6.

TABLE 6
Subject profile of the cohort in which the biomarkers
were assessed (study 3 and 4 combined)

| Characteristics | Control | Colorectal cancer |
|---|---|---|
| Number | 149 | 193 |

Gender Number

| | | |
|---|---|---|
| Female | 58 | 84 |
| Male | 91 | 109 |
| Median age, yrs (range) | 70 (36-89) | 67 (25-93) |

Cancer Stage

| | |
|---|---|
| I | 48 |
| II | 62 |
| III | 61 |
| IV | 22 |

Serum samples from the subjects mentioned in Table 6 were obtained from the Victorian Cancer Biobank. Samples were prepared according to rigorous standard operating protocols as described in https://viccancerbiobank.org.au/for-researchers/quality-assurance/). Concentrations of the biomarkers were measured in each serum sample using commercially available ELISA kits as described in Table 3 above.

Combinations of two to ten biomarkers, differentially weighted to provide the best resolution possible between case and control samples for each combination, were identified using logistic regression as described above. The sensitivity values in Tables 7-15 below are ten-fold cross validated. The top ten performing biomarkers for combinations of two to ten biomarkers are shown in Tables 7 to 15 below.

TABLE 7
Ten biomarker combinations which differentiate colorectal cancer
subjects (both genders) from controls at 95% specificity.

| | | | | | | | | | | Cross- |
|---|---|---|---|---|---|---|---|---|---|---|
| | | | | | | | | | | validated |
| | | | | | | | | | | sensitivity |
| BM1 | BM2 | BM3 | BM4 | BM5 | BM6 | BM7 | BM8 | BM9 | BM10 | (%) |
| BDNF | M2PK | DKK3 | TGFBETA1 | IGFBP2 | LIPOCALIN | TNFALPHA | MIP1B | M65 | IGFII | 85.6 |
| BDNF | M2PK | DKK3 | TGFBETA1 | IGFBP2 | LIPOCALIN | MAC2BP | MIP1B | MMP1 | TIMP1 | 85.2 |
| BDNF | M2PK | DKK3 | TGFBETA1 | IGFBP2 | LIPOCALIN | MAC2BP | MIP1B | MMP1 | MMP7 | 85.1 |
| BDNF | M2PK | DKK3 | TGFBETA1 | IGFBP2 | LIPOCALIN | TNFALPHA | MMP1 | M65 | IGFII | 85 |
| BDNF | M2PK | DKK3 | IL8 | IGFBP2 | LIPOCALIN | TNFALPHA | MMP1 | M65 | MAC2BP | 84.5 |
| BDNF | M2PK | DKK3 | MMP7 | IGFBP2 | LIPOCALIN | IL6 | MIP1B | M65 | MAC2BP | 84.4 |
| BDNF | M2PK | DKK3 | TGFBETA1 | IGFBP2 | LIPOCALIN | TNFALPHA | TIMP1 | M65 | IGFII | 84.4 |
| BDNF | M2PK | DKK3 | TGFBETA1 | IGFBP2 | LIPOCALIN | TIMP1 | MMP1 | M65 | IGFII | 84.4 |
| BDNF | M2PK | DKK3 | TGFBETA1 | IGFBP2 | LIPOCALIN | TNFALPHA | MMP1 | M65 | MMP7 | 84.3 |
| BDNF | M2PK | DKK3 | TGFBETA1 | IGFBP2 | LIPOCALIN | TNFALPHA | TIMP1 | M65 | MMP7 | 84.2 |

TABLE 8
Nine biomarker combinations which differentiate colorectal cancer
subjects (both genders) from controls at 95% specificity.

| | | | | | | | | | Cross- |
|---|---|---|---|---|---|---|---|---|---|
| | | | | | | | | | validated |
| | | | | | | | | | sensitivity |
| BM1 | BM2 | BM3 | BM4 | BM5 | BM6 | BM7 | BM8 | BM9 | (%) |
| BDNF | M2PK | DKK3 | TNFALPHA | IGFBP2 | IL8 | MMP1 | TGFBETA1 | MIP1B | 84.7 |
| BDNF | M2PK | DKK3 | TNFALPHA | IGFBP2 | IL8 | MMP1 | IGFII | MIP1B | 84.7 |
| BDNF | M2PK | DKK3 | TNFALPHA | IGFBP2 | IL8 | MMP1 | MAC2BP | MIP1B | 84.2 |
| BDNF | M2PK | DKK3 | TNFALPHA | IGFBP2 | IL8 | MMP1 | MAC2BP | LIPOCALIN | 84.2 |
| BDNF | M2PK | DKK3 | TNFALPHA | IGFBP2 | LIPOCALIN | MMP1 | IGFII | MIP1B | 84.2 |
| BDNF | M2PK | DKK3 | TIMP1 | IGFBP2 | LIPOCALIN | MMP1 | MAC2BP | MIP1B | 84.2 |
| BDNF | M2PK | DKK3 | IL6 | IGFBP2 | LIPOCALIN | MMP1 | MAC2BP | MMP7 | 84.1 |
| BDNF | M2PK | DKK3 | TGFBETA1 | IGFBP2 | LIPOCALIN | MMP1 | IGFII | MMP7 | 83.9 |
| BDNF | M2PK | DKK3 | TNFALPHA | IGFBP2 | LIPOCALIN | M65 | IGFII | MMP7 | 83.8 |
| BDNF | M2PK | DKK3 | TGFBETA1 | IGFBP2 | LIPOCALIN | MMP1 | MAC2BP | MIP1B | 83.6 |

TABLE 9
Eight biomarker combinations which differentiate colorectal cancer
subjects (both genders) from controls at 95% specificity.

| | | | | | | | | Cross- |
|---|---|---|---|---|---|---|---|---|
| | | | | | | | | validated |
| | | | | | | | | sensitivity |
| BM1 | BM2 | BM3 | BM4 | BM5 | BM6 | BM7 | BM8 | (%) |
| BDNF | M2PK | DKK3 | TNFALPHA | MIP1B | IL8 | MMP1 | IGFBP2 | 84.2 |
| BDNF | M2PK | DKK3 | LIPOCALIN | MAC2BP | IL6 | MMP1 | IGFBP2 | 84.2 |
| BDNF | M2PK | DKK3 | TNFALPHA | MIP1B | MAC2BP | MMP7 | IGFBP2 | 84.1 |
| BDNF | M2PK | DKK3 | TNFALPHA | IGFII | IL8 | MMP1 | M65 | 83.5 |
| BDNF | M2PK | DKK3 | TNFALPHA | MIP1B | LIPOCALIN | TIMP1 | IGFBP2 | 83.1 |
| BDNF | M2PK | DKK3 | TNFALPHA | MIP1B | IL8 | IGFII | IGFBP2 | 83.1 |
| BDNF | M2PK | DKK3 | TGFBETA1 | MIP1B | MAC2BP | MMP1 | IGFBP2 | 83.1 |
| BDNF | M2PK | DKK3 | TNFALPHA | IL13 | LIPOCALIN | IL6 | IGFBP2 | 83.1 |
| BDNF | M2PK | DKK3 | TNFALPHA | TIMP1 | LIPOCALIN | IL6 | IGFBP2 | 83.1 |
| BDNF | M2PK | DKK3 | MMP7 | LIPOCALIN | MAC2BP | MMP1 | IGFBP2 | 83.0 |

TABLE 10
Seven biomarker combinations which differentiate colorectal cancer
subjects (both genders) from controls at 95% specificity.

| | | | | | | | Cross- |
|---|---|---|---|---|---|---|---|
| | | | | | | | validated |
| | | | | | | | sensitivity |
| BM1 | BM2 | BM3 | BM4 | BM5 | BM6 | BM7 | (%) |
| BDNF | M2PK | DKK3 | IGFBP2 | MAC2BP | TNFALPHA | MIP1B | 83.2 |
| BDNF | M2PK | DKK3 | IGFBP2 | MMP7 | TNFALPHA | LIPOCALIN | 83 |
| BDNF | M2PK | DKK3 | M65 | IL8 | TNFALPHA | MMP1 | 83 |
| BDNF | M2PK | DKK3 | IGFBP2 | TIMP1 | TNFALPHA | IGFII | 81.9 |
| BDNF | M2PK | DKK3 | IGFBP2 | MAC2BP | TNFALPHA | MMP7 | 81.9 |
| BDNF | M2PK | DKK3 | IGFBP2 | M65 | TNFALPHA | IGFII | 81.8 |
| BDNF | M2PK | TGFBETA1 | M65 | IL8 | TNFALPHA | MMP7 | 81.6 |
| BDNF | M2PK | DKK3 | IGFBP2 | MAC2BP | MMP1 | MIP1B | 81.5 |
| BDNF | M2PK | DKK3 | IGFBP2 | IL6 | TNFALPHA | LIPOCALIN | 81.5 |
| BDNF | M2PK | TGFBETA1 | IGFBP2 | IL8 | TNFALPHA | LIPOCALIN | 81.4 |

TABLE 11
Six biomarker combinations which differentiate colorectal cancer
subjects (both genders) from controls at 95% specificity.

| | | | | | | Cross- |
|---|---|---|---|---|---|---|
| | | | | | | validated |
| | | | | | | sensitivity |
| BM1 | BM2 | BM3 | BM4 | BM5 | BM6 | (%) |
| BDNF | M2PK | MIP1B | DKK3 | IGFBP2 | TNFALPHA | 80.4 |
| BDNF | M2PK | MIP1B | DKK3 | IGFBP2 | MMP1 | 80.4 |
| BDNF | M2PK | MAC2BP | DKK3 | IGFBP2 | TNFALPHA | 80.4 |
| BDNF | M2PK | LIPOCALIN | DKK3 | IGFBP2 | TNFALPHA | 80.4 |
| BDNF | M2PK | IL6 | DKK3 | IGFBP2 | TNFALPHA | 80.4 |
| BDNF | M2PK | IL8 | DKK3 | LIPOCALIN | TGFBETA1 | 80.4 |
| BDNF | M2PK | IL8 | MIP1B | TGFBETA1 | TNFALPHA | 79.9 |
| BDNF | M2PK | IL8 | MIP1B | IGFII | TNFALPHA | 79.9 |
| BDNF | M2PK | IL8 | M30 | TGFBETA1 | TNFALPHA | 79.9 |
| BDNF | M2PK | IL8 | DKK3 | IGFII | TNFALPHA | 79.9 |

TABLE 12
Five biomarker combinations which differentiate colorectal cancer
subjects (both genders) from controls at 95% specificity.

| | | | | | Cross-validated |
|---|---|---|---|---|---|
| BM1 | BM2 | BM3 | BM4 | BM5 | sensitivity (%) |
| BDNF | M2PK | IL8 | TNFALPHA | MIP1B | 80.5 |
| BDNF | M2PK | IL8 | LIPOCALIN | DKK3 | 80 |
| BDNF | M2PK | IL8 | TGFBETA1 | DKK3 | 79.9 |
| BDNF | M2PK | IL8 | TNFALPHA | TGFBETA1 | 79.3 |
| BDNF | M2PK | IGFBP2 | TNFALPHA | DKK3 | 79.3 |
| BDNF | M2PK | IL8 | MMP7 | DKK3 | 79.2 |
| BDNF | M2PK | IL8 | TNFALPHA | DKK3 | 78.9 |
| BDNF | M2PK | IL8 | IGFBP2 | MIP1B | 78.8 |
| BDNF | M2PK | IL8 | MAC2BP | DKK3 | 78.4 |
| BDNF | M2PK | IL8 | TNFALPHA | MAC2BP | 77.8 |

TABLE 13
Four biomarker combinations which differentiate colorectal cancer
subjects (both genders) from controls at 95% specificity.

| | | | | Cross-validated |
|---|---|---|---|---|
| BM1 | BM2 | BM3 | BM4 | sensitivity (%) |
| BDNF | M2PK | IL8 | TNFALPHA | 77.8 |
| BDNF | M2PK | IL8 | MIP1B | 77.3 |
| BDNF | M2PK | IL8 | DKK3 | 77.3 |
| BDNF | M2PK | MMP1 | DKK3 | 76.8 |
| BDNF | M2PK | IL8 | IGFBP2 | 76.1 |
| BDNF | M2PK | IL8 | TIMP1 | 75.5 |
| BDNF | M2PK | MMP7 | TNFALPHA | 75.4 |
| BDNF | M2PK | IL8 | MMP1 | 75.1 |
| BDNF | M2PK | IGFBP2 | TNFALPHA | 75 |
| BDNF | M2PK | IL8 | IGFII | 75 |

TABLE 14
Three biomarker combinations which differentiate colorectal cancer
subjects (both genders) from controls at 95% specificity.

| | | | Cross-validated |
|---|---|---|---|
| BM1 | BM2 | BM3 | sensitivity (%) |
| BDNF | M2PK | IL8 | 74.6 |
| BDNF | M2PK | TNFALPHA | 72.4 |
| BDNF | M2PK | DKK3 | 71.4 |
| BDNF | M2PK | IGFBP2 | 68.5 |
| BDNF | M2PK | MAC2BP | 68.1 |

TABLE 15
Two biomarker combinations which differentiate colorectal cancer
subjects (both genders) from controls at 95% specificity.

| | | Cross-validated |
|---|---|---|
| BM1 | BM2 | sensitivity (%) |
| BDNF | M2PK | 63.8 |

From Tables 7-15 it will be apparent that the highest sensitivities were obtained when more biomarkers were included in the panel. For panels within the four to ten biomarker panel size classes, sensitivity declines observed within the top ten models for any particular biomarker panel numerical class were very modest ranging from 1.2% for the 8 biomarker panel to 3.8% for the 4 biomarker panel. For biomarker panels comprising seven to ten biomarkers and four to six biomarkers, the range of sensitivity values observed for the top ten panels overlapped between adjacent biomarker panel size classes. The highest sensitivity recorded (for a mix of male and female genders) was 85.6% at 95% specificity for a ten biomarker panel comprising BDNF, M2PK, DKK3, TGFBETA, IGFBP2, LIPOCALIN, TNFα, MIP1β, M65 and IGFII.

BDNF and M2PK were present in the top performing panel in each biomarker panel size class. In the top ten six to ten biomarker panel size classes, other prominently observed biomarkers included IGFBP2, DKK3 and TNFα. Between four to eight biomarkers, IL8 also appeared to be well represented in the top ten models.

To identify biomarkers most frequently found in a broader set of biomarker panels showing strong resolution between sera from persons (male and female combined) with and without advanced colorectal neoplasia, the frequency with which individual biomarkers were represented in the top 100 models (or top models producing a sensitivity of ≥75% at 95% specificity) for biomarker panels ten to three respectively were plotted.

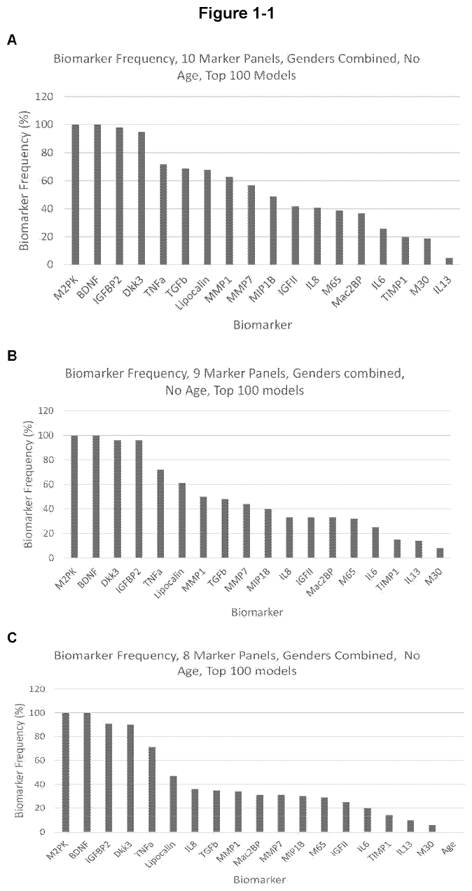

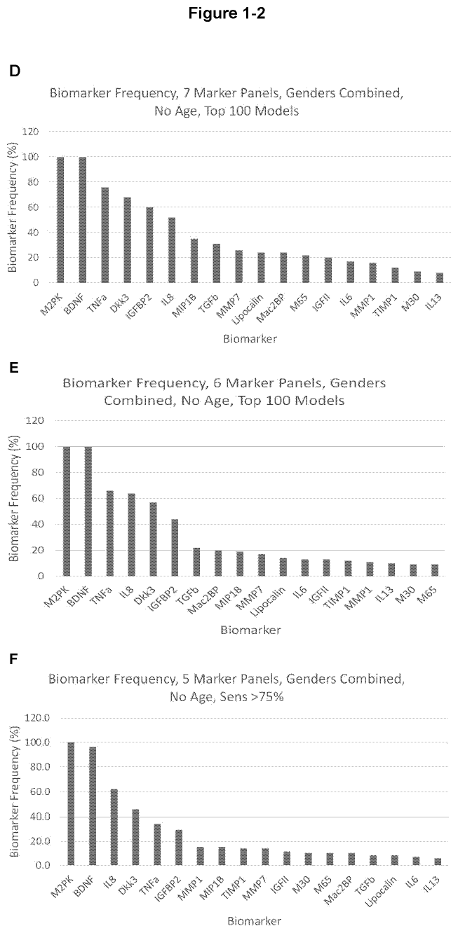

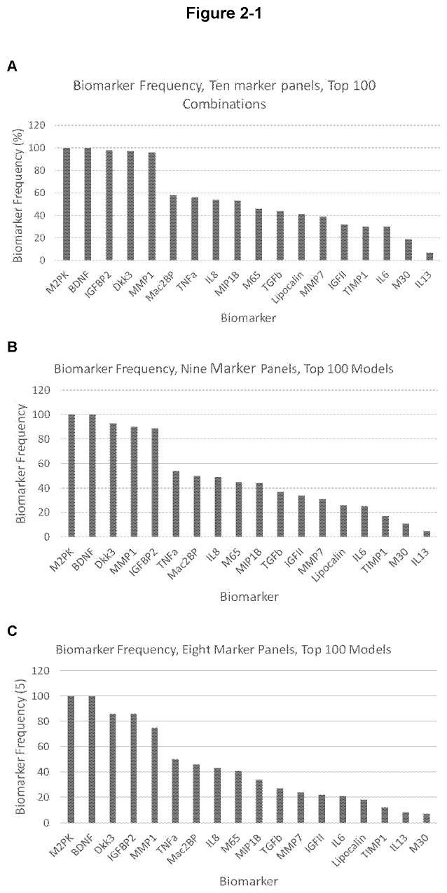

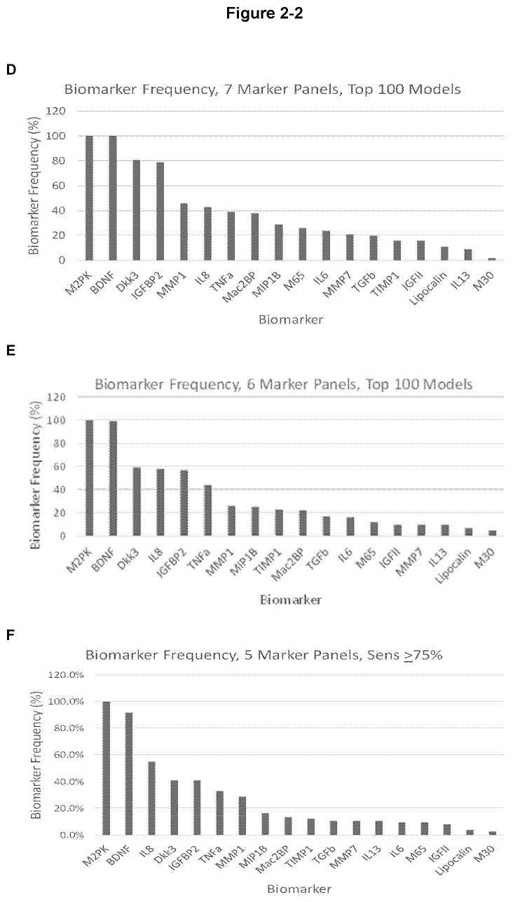

Results obtained for both genders combined where no age term was included in the modelling are shown in FIGS. 1-1 and 1-2. For all biomarker panels, M2PK and BDNF were present.

For panels of eight to ten biomarkers, other biomarkers (present in >50% of this broader selection of high performing panels) included DKK3, IGFBP2, and TNFα, For panels of four to seven biomarkers, IL8 emerged as a prominent marker as TNFα and IGFBP2 began to wane while still contributing to substantial proportions of the strongly performing biomarker panels.

Without wishing to be bound by theory, the present inventors have found that the inclusion of BDNF in a biomarker panel provides an unexpected improvement in the test sensitivity at 95% specificity and/or the cross-validated sensitivity at 95% specificity compared to earlier disclosed biomarker combinations. For example, WO 2012/006681 discloses a method for diagnosing or detecting colorectal cancer in a subject. In this application, the highest ranking three biomarker combination was DKK3, M2PK and IGFBP2 had a test sensitivity of 72.9% and cross-validated sensitivity of 70.8%, both at 95% specificity. The present inventors have demonstrated that the inclusion of BDNF in this biomarker panel (i.e. BDNF, DKK3, M2PK and IGFBP2) improves the test sensitivity and cross-validated sensitivity at 95% specificity to 73.9% and 74.5% respectively. Similarly, the highest ranking four biomarker combination in WO 2012/006681 was DKK3, M2PK, IGFBP2 and Mac2BP which had a test sensitivity of 68.8% and cross-validated sensitivity of 69.8%, both at 95% specificity. The present inventors have demonstrated that the inclusion of BDNF in this biomarker panel (BDNF, DKK3, M2PK, IGFBP2 and Mac2BP) improves the test sensitivity and cross-validated sensitivity at 95% specificity to 77.7% and 76.6% respectively. Finally, the four biomarker combination DKK3, M2PK, IGFBP2 and TIMP1 was reported to have a test sensitivity of 61.5% and cross-validated sensitivity of 53.1%, both at 95% specificity in WO2012/006681. The present inventors have demonstrated that the inclusion of BDNF in this biomarker panel (i.e. BDNF, DKK3, M2PK, IGFBP2 and TIMP1) significantly improves the test sensitivity and cross-validated sensitivity at 95% specificity to 77% and 76%, respectively.

Enhanced performance over of biomarker combinations recited in WO 2012/006681 resulting from inclusion of BDNF was observed when the top performing biomarker combinations were considered in the various biomarker panels. For example the top performing seven-marker panel, identified when BDNF was included in the modelling process, contained BDNF and displayed an internally cross validated sensitivity for all-stage CRC (age and/or gender not included) of 83.2% at 95% specificity compared to 69% for the top-performing, internally cross validated, seven-biomarker panel model described in WO 2012/006681 where BDNF was not included. Similarly for three, four and five biomarker panels including BDNF, top performing models in each class showed sensitivities of 74.6%, 77.8% and 80.5% respectively at 95% specificity compared with 70.8%, 69.8% and 70.8% for top performing models where BDNF was not included. Indeed, surprisingly, BDNF was included in all or the vast majority of the 100 top performing models (or models producing a sensitivity at 95% specificity>75%) in based on panels of 5-10 biomarkers (see FIGS. 2-1 and 2-2).

### Example 3 The Impact of Adding Demographic Data—Age

Age is the highest risk factor for CRC. The risk of developing colorectal cancer increases dramatically from age 50 yrs. The inventors therefore assessed, using logistic regression analysis, whether including age in conjunction with multiple biomarker combinations could modify the sensitivity for the detection of CRC achieved with biomarker combinations. Tables 16-24 below show the sensitivity at 95% specificity of the top ten performing two to ten biomarker panels when a weighed age in years is added as an additional biomarker to the analysis. Examples of the top ten performing two to ten biomarker combinations, plus age, are shown in Tables 16-24.

TABLE 16
Ten biomarker combinations plus age which differentiate colorectal
cancer subjects (both genders) from controls at 95% specificity.

| | | | | | | | | | | Cross- |
|---|---|---|---|---|---|---|---|---|---|---|
| | | | | | | | | | | validated |
| | | | | | | | | | | Sensitivity |
| BM1 | BM2 | BM3 | BM4 | BM5 | BM6 | BM7 | BM8 | BM9 | BM10 | (%) |
| BDNF | M2PK | DKK3 | TNFALPHA | IGFBP2 | IL8 | MMP1 | MAC2BP | MIP1B | MMP7 | 86.8 |
| BDNF | M2PK | DKK3 | TNFALPHA | IGFBP2 | IL8 | LIPOCALIN | MMP1 | M65 | MIP1B | 86.7 |
| BDNF | M2PK | DKK3 | IL6 | IGFBP2 | LIPOCALIN | MMP1 | IGFII | MAC2BP | MMP7 | 86.7 |
| BDNF | M2PK | DKK3 | TNFALPHA | IGFBP2 | TIMP1 | IL8 | MMP1 | MAC2BP | MIP1B | 86.3 |
| BDNF | M2PK | DKK3 | TNFALPHA | IGFBP2 | IL8 | MMP1 | M65 | MAC2BP | MIP1B | 86.2 |
| BDNF | M2PK | DKK3 | TNFALPHA | IGFBP2 | IL8 | LIPOCALIN | MMP1 | M65 | MAC2BP | 86.2 |
| BDNF | M2PK | DKK3 | TNFALPHA | IGFBP2 | IL8 | TGFBETA1 | MMP1 | LIPOCALIN | MIP1B | 85.8 |
| BDNF | M2PK | DKK3 | TIMP1 | IGFBP2 | IL6 | LIPOCALIN | MMP1 | MAC2BP | MIP1B | 85.8 |
| BDNF | M2PK | DKK3 | TNFALPHA | IGFBP2 | TIMP1 | IL8 | MMP1 | IGFII | MIP1B | 85.7 |
| BDNF | M2PK | DKK3 | TNFALPHA | IGFBP2 | IL8 | IGFII | MMP1 | M65 | MIP1B | 85.6 |

TABLE 17
Nine biomarker combinations plus age which differentiate colorectal
cancer subjects (both genders) from controls at 95% specificity.

| | | | | | | | | | Cross- |
|---|---|---|---|---|---|---|---|---|---|
| | | | | | | | | | validated |
| | | | | | | | | | Sensitivity |
| BM1 | BM2 | BM3 | BM4 | BM5 | BM6 | BM7 | BM8 | BM9 | (%) |
| BDNF | M2PK | DKK3 | TNFALPHA | IGFBP2 | IL8 | MMP1 | MAC2BP | MIP1B | 87.0 |
| BDNF | M2PK | DKK3 | TGFBETA1 | IGFBP2 | LIPOCALIN | M65 | IGFII | MMP7 | 86.0 |
| BDNF | M2PK | DKK3 | IL6 | IGFBP2 | TGFBETA1 | MMP1 | MAC2BP | MIP1B | 85.8 |
| BDNF | M2PK | DKK3 | TNFALPHA | IGFBP2 | IL8 | MMP1 | IGFII | MIP1B | 85.8 |
| BDNF | M2PK | DKK3 | IL6 | IGFBP2 | LIPOCALIN | TGFBETA1 | MMP1 | MAC2BP | 85.8 |
| BDNF | M2PK | DKK3 | IL6 | IGFBP2 | LIPOCALIN | MMP1 | MAC2BP | MMP7 | 85.7 |
| BDNF | M2PK | DKK3 | TNFALPHA | IGFBP2 | LIPOCALIN | MMP1 | IL8 | M65 | 85.6 |
| BDNF | M2PK | DKK3 | TNFALPHA | IGFBP2 | TIMP1 | MMP1 | IL8 | MIP1B | 85.2 |
| BDNF | M2PK | DKK3 | TNFALPHA | IGFBP2 | IL8 | MMP1 | M65 | IGFII | 85.1 |
| BDNF | M2PK | DKK3 | IL6 | IGFBP2 | LIPOCALIN | M65 | MIP1B | MMP7 | 84.9 |

TABLE 18
Eight biomarker combinations plus age which differentiate colorectal
cancer subjects (both genders) from controls at 95% specificity.

| | | | | | | | | Cross- |
|---|---|---|---|---|---|---|---|---|
| | | | | | | | | validated |
| | | | | | | | | Sensitivity |
| BM1 | BM2 | BM3 | BM4 | BM5 | BM6 | BM7 | BM8 | (%) |
| BDNF | M2PK | DKK3 | TNFALPHA | IL8 | IGFBP2 | MMP1 | M65 | 85.1 |
| BDNF | M2PK | DKK3 | IL6 | LIPOCALIN | IGFBP2 | MMP1 | MAC2BP | 84.8 |
| BDNF | M2PK | DKK3 | MAC2BP | TGFBETA1 | IGFBP2 | MMP1 | M65 | 84.4 |
| BDNF | M2PK | DKK3 | IL6 | TGFBETA1 | IGFBP2 | MMP1 | MAC2BP | 84.2 |
| BDNF | M2PK | DKK3 | TNFALPHA | IL6 | IL8 | MMP1 | M65 | 84.1 |
| BDNF | M2PK | DKK3 | TNFALPHA | IGFII | IL8 | MMP1 | M65 | 84.1 |
| BDNF | M2PK | DKK3 | TNFALPHA | LIPOCALIN | IGFBP2 | MAC2BP | MMP7 | 84.1 |
| BDNF | M2PK | DKK3 | TNFALPHA | MIP1B | IGFBP2 | MAC2BP | MMP7 | 84.1 |
| BDNF | M2PK | DKK3 | IGFII | TIMP1 | IGFBP2 | MMP1 | M65 | 83.9 |
| BDNF | M2PK | DKK3 | IGFII | TGFBETA1 | IGFBP2 | MMP1 | M65 | 83.9 |

TABLE 19
Seven biomarker combinations plus age which differentiate colorectal
cancer subjects (both genders) from controls at 95% specificity.

| | | | | | | | Cross- |
|---|---|---|---|---|---|---|---|
| | | | | | | | validated |
| | | | | | | | Sensitivity |
| BM1 | BM2 | BM3 | BM4 | BM5 | BM6 | BM7 | (%) |
| BDNF | M2PK | DKK3 | LIPOCALIN | MAC2BP | IGFBP2 | MMP1 | 84.2 |
| BDNF | M2PK | DKK3 | TNFALPHA | IL8 | M65 | MMP1 | 84.1 |
| BDNF | M2PK | DKK3 | MMP7 | MAC2BP | IGFBP2 | MMP1 | 84.1 |
| BDNF | M2PK | DKK3 | TNFALPHA | IL8 | IGFBP2 | MMP1 | 83.7 |
| BDNF | M2PK | DKK3 | IL6 | MAC2BP | IGFBP2 | MMP1 | 83.7 |
| BDNF | M2PK | DKK3 | M65 | MAC2BP | IGFBP2 | MMP1 | 83.4 |
| BDNF | M2PK | DKK3 | MIP1B | MAC2BP | IGFBP2 | MMP1 | 83.2 |
| BDNF | M2PK | DKK3 | M30 | MAC2BP | IGFBP2 | MMP1 | 82.6 |
| BDNF | M2PK | DKK3 | IL8 | MIP1B | IGFBP2 | MMP1 | 82.6 |
| BDNF | M2PK | DKK3 | IL6 | MAC2BP | IGFBP2 | MMP7 | 82.4 |

TABLE 20
Six biomarker combinations plus age which differentiate colorectal
cancer subjects (both genders) from controls at 95% specificity.

| | | | | | | Cross- |
|---|---|---|---|---|---|---|
| | | | | | | validated |
| | | | | | | Sensitivity |
| BM1 | BM2 | BM3 | BM4 | BM5 | BM6 | (%) |
| BDNF | M2PK | DKK3 | MAC2BP | IGFBP2 | MMP1 | 83.2 |
| BDNF | M2PK | TNFALPHA | TGFBETA1 | IL8 | MIP1B | 81.0 |
| BDNF | M2PK | DKK3 | IL6 | IGFBP2 | MMP1 | 81.0 |
| BDNF | M2PK | DKK3 | TNFALPHA | IL8 | M65 | 80.8 |
| BDNF | M2PK | DKK3 | IL6 | IGFBP2 | MAC2BP | 80.4 |
| BDNF | M2PK | DKK3 | MIP1B | IGFBP2 | MAC2BP | 80.4 |
| BDNF | M2PK | DKK3 | IL8 | IGFBP2 | MMP1 | 80.4 |
| BDNF | M2PK | TNFALPHA | IL8 | MIP1B | MAC2BP | 80.0 |
| BDNF | M2PK | TNFALPHA | IL8 | MMP1 | MAC2BP | 80.0 |
| BDNF | M2PK | DKK3 | IL6 | IGFBP2 | MIP1B | 79.9 |

TABLE 21
Five biomarker combinations plus age which differentiate colorectal
cancer subjects (both genders) from controls at 95% specificity.

| | | | | | Cross-validated |
|---|---|---|---|---|---|
| BM1 | BM2 | BM3 | BM4 | BM5 | Sensitivity (%) |
| BDNF | M2PK | TNFALPHA | IL8 | MIP1B | 80.0 |
| BDNF | M2PK | DKK3 | IL6 | IGFBP2 | 79.9 |
| BDNF | M2PK | DKK3 | MMP1 | MAC2BP | 79.5 |
| BDNF | M2PK | TIMP1 | MMP1 | MIP1B | 79.3 |
| BDNF | M2PK | DKK3 | TIMP1 | IGFBP2 | 79.2 |
| BDNF | M2PK | DKK3 | LIPOCALIN | IGFBP2 | 78.8 |
| BDNF | M2PK | TNFALPHA | IL8 | TGFBETA1 | 78.3 |
| BDNF | M2PK | DKK3 | MAC2BP | IGFBP2 | 78.3 |
| BDNF | M2PK | DKK3 | TNFALPHA | IL8 | 77.8 |
| BDNF | M2PK | TNFALPHA | IL6 | IL8 | 77.8 |

TABLE 22
Four biomarker combinations plus age which differentiate colorectal
cancer subjects (both genders) from controls at 95% specificity.

| | | | | Cross-validated |
|---|---|---|---|---|
| BM1 | BM2 | BM3 | BM4 | sensitivity (%) |
| BDNF | M2PK | IL8 | DKK3 | 77.8 |
| BDNF | M2PK | IL8 | MIP1B | 76.8 |
| BDNF | M2PK | IL8 | TNFALPHA | 76.2 |
| BDNF | M2PK | MMP1 | DKK3 | 75.7 |
| BDNF | M2PK | TNFALPHA | IGFBP2 | 75.5 |
| BDNF | M2PK | IL8 | IGFBP2 | 75 |
| BDNF | M2PK | TNFALPHA | DKK3 | 74.6 |
| BDNF | M2PK | IL8 | IL13 | 74.5 |
| BDNF | M2PK | IL8 | IGFII | 74.5 |

TABLE 23
Three biomarker combinations plus age which differentiate colorectal
cancer subjects (both genders) from controls at 95% specificity.

| | | | Cross-validated |
|---|---|---|---|
| BM1 | BM2 | BM3 | sensitivity (%) |
| BDNF | M2PK | IL8 | 72.4 |
| BDNF | M2PK | DKK3 | 71.4 |
| BDNF | M2PK | TNFALPHA | 65.9 |

TABLE 24
Two biomarker combinations plus age which differentiate colorectal
cancer subjects (both genders) from controls at 95% specificity.

| | | Cross-validated |
|---|---|---|
| BM1 | BM2 | sensitivity (%) |
| BDNF | M2PK | 56.8 |

Similarly to biomarker combinations in the absence of age, when age was included the highest sensitivities were obtained when there were more biomarkers in the panel. Within the top ten four to ten biomarker panels plus age, sensitivity declines observed within any particular biomarker panel size class were again modest, ranging from 1.2% for the 10 biomarker panel size class to 3.3% for the 4 and 6 biomarker panel size classes. For four to ten biomarker panels plus age, the ranges of sensitivity values observed for the top ten biomarker panels overlapped between adjacent panel size classes. The highest sensitivity recorded for biomarkers plus age, both genders, at 95% specificity was 87% with a nine biomarker panel.

For panels of six to ten biomarkers, the top model that included age showed marginally higher sensitivity values than their counterparts that did not include age. The biomarker compositions for the top performing panel in each biomarker panel size class differed somewhat between the models that included and didn't include age with the exception of the five biomarker panel. Importantly, however, those biomarkers that were most prominent in the top ten biomarker panel size classes in the absence of age were conserved in the equivalent panels where age was included. In particular, BDNF and M2PK (tumour form) were present in all models. DKK3 and IGFBP2 were prominent, particularly in the six to ten biomarker panel size classes.

Also appearing frequently in many top biomarker panels were IL8, TNFα and MMP1. IGFBP2 and MMP1 were more commonly represented in panels of six to ten biomarkers but IL8 and TNFα were prominent in all biomarker panels. MIP1β was strongly represented in top models for eight to ten biomarker panels but its frequency waned somewhat in top models comprising six or fewer biomarkers.

The frequencies with which individual biomarkers were represented in the top 100 biomarker models (or top models producing a sensitivity of ≥75% at 95% specificity) for ten to five biomarker panels respectively based on data from both genders combined where age was included in the modelling are shown in FIGS. 2-1 and 2-2. For almost all biomarker panels, M2PK and BDNF were present in all panels in this broader selection of top performing models for genders combined when age was included. The exception was five biomarker panels where 8% of the top 100 models did not contain BDNF. For eight to ten biomarker panels other prominent biomarkers (present in >50% of this broader selection of high performing models) included DKK3, IGFBP2, MMP1 and TNFα. For four to seven biomarker panels, IL8 remained prominent though lower than M2PK and BDNF. TNFα, DKK3, MMP1 and IGFBP2 panel inclusion rates fell below 50% in these panels but continued to be included in a higher proportion of the strongly performing panels than other biomarkers.

It will be understood by the person skilled in the art that there is an inverse relationship between sensitivity and specificity. Thus when specificity is reduced, the sensitivity of the test will generally increase. Operationally, circumstances exist where a higher sensitivity may be required and a lower specificity is acceptable. For example, while there are no two marker panels plus age that differentiate cancer from control serum samples with >75% sensitivity at 95% specificity, if the specificity is reduced to 90% a model comprising M2PK, BDNF and age yielded a cross-validated sensitivity of 77.8%.

### Example 4 Impact of Gender (Males and Females Analysed Separately)

It is known that males are more prone to develop and to die from colorectal cancer. In 2015, of the 15,604 new cases of colorectal cancer diagnosed in Australia, 7,031 were females while 8,573 were males. In 2019, of the estimated 5,597 deaths in that year resulting from colorectal cancer, 2,588 were females while 3009 were males. Also in 2019, it was estimated that the risk of an individual Australian being diagnosed with colorectal cancer by their 85th birthday was 1 in 14 (1 in 12 males and 1 in 17 females) (https://bowel-cancer.canceraustralia.gov.au/statistics). These gender biases in colorectal cancer risk statistics are also reflected in the positivity rates by gender when assessed by FIT in the Australia's National Bowel Cancer Screening Program based on data from 2017 (8.8% for males, 7.1% for females) (Australian Institute of Health and Welfare 2019. National Bowel Cancer Screening Program: monitoring report 2019. Cancer series no. 125. Cat. no. CAN 125. Canberra: AIHW). While these patterns for increased frequency of colorectal cancer in males relative to females were also reflected in a study of a new methylation diagnostic test for colorectal cancer, gender was not a predictor of positivity for that test per se (Pedersen et al. Evaluation of an assay for methylated BCAT1 and IKZF1 in plasma for detection of colorectal neoplasia. BMC Cancer (2015) 15:654 DOI 10.1186/s12885-015-1674-2).

The best performing 10, 9, 8, 7, 6, 5, 4, 3 and 2 biomarker panels including BDNF but not age for both sexes combined, at 95% specificity, were illustrated in Tables 7-15 above with top observed sensitivities of 85.6%, 84.7%, 84.2%, 83.2%, 80.4%, 80.5%, 77.8%, 74.6% and 63.8% respectively.

To understand whether these panels were working equally well for both sexes, the same core biomarker concentration data from the 193 colorectal cancer, 149 healthy control cohort were separated into male-only and female-only case/control cohorts. Tables 25-60 below show best performing two to ten biomarker panels, identified by logistic regression and 10-fold cross validation performed independently on biomarker data derived from these female or male cohorts.

Examples of two to ten biomarker panels for females that included BDNF are shown in Tables 25-33. Examples of two to ten biomarker panels for females that include BDNF when age was also considered are shown in Table 34-42. The top ten biomarker combinations are shown for each Table below.

TABLE 25
Ten biomarker combinations which differentiate colorectal
cancer subjects (female) from controls at 95% specificity.

| | | | | | | | | | | Cross |
|---|---|---|---|---|---|---|---|---|---|---|
| | | | | | | | | | | Validated |
| | | | | | | | | | | Sensitivity |
| BM1 | BM2 | BM3 | BM4 | BM5 | BM6 | BM7 | BM8 | BM9 | BM10 | (%) |
| BDNF | M2PK | IL8 | M65 | MMP1 | MIP1B | M30 | IL6 | DKK3 | IL13 | 93.3 |
| BDNF | M2PK | IL8 | M65 | MMP1 | MIP1B | M30 | IL6 | IL13 | TIMP1 | 93.2 |
| BDNF | M2PK | IL8 | M65 | MMP1 | MIP1B | M30 | TGFBETA1 | TNFALPHA | MAC2BP | 92.1 |
| BDNF | M2PK | IL8 | M65 | MMP1 | MIP1B | M30 | TGFBETA1 | TNFALPHA | DKK3 | 92.1 |
| BDNF | M2PK | IL8 | M65 | MMP1 | MIP1B | M30 | IL6 | TGFBETA1 | MAC2BP | 92.1 |
| BDNF | M2PK | IL8 | M65 | MMP1 | MIP1B | M30 | TGFBETA1 | MAC2BP | IGFII | 92.1 |
| BDNF | M2PK | IL8 | M65 | MMP1 | MIP1B | M30 | TGFBETA1 | MAC2BP | DKK3 | 92.1 |
| BDNF | M2PK | IL8 | M65 | MMP1 | MIP1B | M30 | IL6 | TGFBETA1 | IGFII | 92.1 |
| BDNF | M2PK | IL8 | M65 | MMP1 | MIP1B | M30 | IL6 | TGFBETA1 | DKK3 | 92.1 |
| BDNF | M2PK | IL8 | M65 | MMP1 | MIP1B | M30 | TGFBETA1 | DKK3 | IGFII | 92.1 |

TABLE 26
Nine biomarker combinations which differentiate colorectal
cancer subjects (female) from controls at 95% specificity.

| | | | | | | | | | Cross |
|---|---|---|---|---|---|---|---|---|---|
| | | | | | | | | | Validated |
| | | | | | | | | | Sensitivity |
| BM1 | BM2 | BM3 | BM4 | BM5 | BM6 | BM7 | BM8 | BM9 | (%) |
| BDNF | M2PK | IL8 | M65 | MMP1 | MIP1B | M30 | IL6 | IL13 | 93.3 |
| BDNF | M2PK | IL8 | M65 | MMP1 | MIP1B | M30 | TGFBETA1 | TNFALPHA | 92.1 |
| BDNF | M2PK | IL8 | M65 | MMP1 | MIP1B | M30 | TGFBETA1 | MAC2BP | 92.1 |
| BDNF | M2PK | IL8 | M65 | MMP1 | MIP1B | M30 | IL6 | TGFBETA1 | 92.1 |
| BDNF | M2PK | IL8 | M65 | MMP1 | MIP1B | M30 | TGFBETA1 | IGFII | 92.1 |
| BDNF | M2PK | IL8 | M65 | MMP1 | MIP1B | M30 | TGFBETA1 | DKK3 | 92.1 |
| BDNF | M2PK | IL8 | M65 | MMP1 | MIP1B | M30 | TGFBETA1 | IL13 | 92 |
| BDNF | M2PK | IL8 | M65 | MMP1 | MIP1B | M30 | DKK3 | IL13 | 92 |
| BDNF | M2PK | IL8 | M65 | MMP1 | MIP1B | M30 | TGFBETA1 | MMP7 | 92 |
| BDNF | M2PK | IL8 | M65 | MMP1 | TNFALPHA | MAC2BP | IGFII | TIMP1 | 92 |

TABLE 27
Eight biomarker combinations which differentiate colorectal
cancer subjects (female) from controls at 95% specificity.

| | | | | | | | | Cross |
|---|---|---|---|---|---|---|---|---|
| | | | | | | | | Validated |
| | | | | | | | | Sensitivity |
| BM1 | BM2 | BM3 | BM4 | BM5 | BM6 | BM7 | BM8 | (%) |
| BDNF | M2PK | IL8 | M65 | MMP1 | MIP1B | M30 | TGFBETA1 | 92.1 |
| BDNF | M2PK | IL8 | M65 | MMP1 | TNFALPHA | MAC2BP | TIMP1 | 92 |
| BDNF | M2PK | M65 | MMP1 | MIP1B | TNFALPHA | MAC2BP | LIPOCALIN | 90.8 |
| BDNF | M2PK | IL8 | M65 | MMP1 | TGFBETA1 | TNFALPHA | MAC2BP | 90.8 |
| BDNF | M2PK | IL8 | M65 | MMP1 | TNFALPHA | MAC2BP | IGFII | 90.8 |
| BDNF | M2PK | IL8 | M65 | MMP1 | M30 | TGFBETA1 | MAC2BP | 90.8 |
| BDNF | M2PK | IL8 | M65 | MMP1 | M30 | IL6 | TGFBETA1 | 90.8 |
| BDNF | M2PK | IL8 | M65 | MMP1 | M30 | TGFBETA1 | DKK3 | 90.8 |
| BDNF | M2PK | IL8 | MMP1 | TGFBETA1 | IGFII | LIPOCALIN | IGFBP2 | 90.8 |
| BDNF | M2PK | IL8 | M65 | MMP1 | M30 | DKK3 | IGFII | 90.8 |

TABLE 28
Seven biomarker combinations which differentiate colorectal
cancer subjects (female) from controls at 95% specificity.

| | | | | | | | Cross- |
|---|---|---|---|---|---|---|---|
| | | | | | | | validated |
| | | | | | | | sensitivity |
| BM1 | BM2 | BM3 | BM4 | BM5 | BM6 | BM7 | (%) |
| BDNF | M2PK | IL8 | M65 | MAC2BP | MMP1 | TNFALPHA | 90.8 |
| BDNF | M2PK | IL8 | M65 | LIPOCALIN | MMP1 | TNFALPHA | 90.8 |
| BDNF | M2PK | IL8 | M65 | TGFBETA1 | MMP1 | M30 | 90.8 |
| BDNF | M2PK | IL8 | M65 | MAC2BP | MMP1 | IGFII | 90.8 |
| BDNF | M2PK | IL8 | IGFBP2 | LIPOCALIN | MMP1 | IGFII | 90.8 |
| BDNF | M2PK | IL8 | M65 | IL13 | MIP1B | M30 | 90.7 |
| BDNF | M2PK | IL8 | M65 | IL13 | M30 | TNFALPHA | 90.7 |
| BDNF | M2PK | IL8 | M65 | IL13 | MAC2BP | M30 | 90.7 |
| BDNF | M2PK | IL8 | M65 | IL13 | IL6 | M30 | 90.7 |
| BDNF | M2PK | IL8 | M65 | IL13 | IGFII | M30 | 90.7 |

TABLE 29
Six biomarker combinations which differentiate colorectal
cancer subjects (female) from controls at 95% specificity.

| | | | | | | Cross- |
|---|---|---|---|---|---|---|
| | | | | | | validated |
| | | | | | | sensitivity |
| BM1 | BM2 | BM3 | BM4 | BM5 | BM6 | (%) |
| BDNF | M2PK | IL8 | IL13 | M65 | M30 | 90.7 |
| BDNF | M2PK | IL8 | MMP1 | TGFBETA1 | TNFALPHA | 89.7 |
| BDNF | M2PK | IL8 | MAC2BP | TGFBETA1 | TNFALPHA | 89.7 |
| BDNF | M2PK | IL8 | IL6 | TGFBETA1 | TNFALPHA | 89.7 |
| BDNF | M2PK | IL8 | MMP1 | MAC2BP | TNFALPHA | 89.7 |
| BDNF | M2PK | IL8 | MMP1 | TGFBETA1 | MAC2BP | 89.7 |
| BDNF | M2PK | IL8 | MMP1 | TGFBETA1 | DKK3 | 89.7 |
| BDNF | M2PK | IL8 | IL6 | TGFBETA1 | MAC2BP | 89.7 |
| BDNF | M2PK | IL8 | MMP1 | DKK3 | MAC2BP | 89.7 |
| BDNF | M2PK | IL8 | IL13 | TGFBETA1 | MIP1B | 89.6 |

TABLE 30
Five biomarker combinations which differentiate colorectal
cancer subjects (female) from controls at 95% specificity.

| | | | | | Cross-validated |
|---|---|---|---|---|---|
| BM1 | BM2 | BM3 | BM4 | BM5 | sensitivity (%) |
| BDNF | M2PK | IL8 | TGFBETA1 | TNFALPHA | 89.7 |
| BDNF | M2PK | IL8 | TNFALPHA | MMP1 | 89.7 |
| BDNF | M2PK | IL8 | TGFBETA1 | MMP1 | 89.7 |
| BDNF | M2PK | IL8 | TGFBETA1 | MAC2BP | 89.7 |
| BDNF | M2PK | IL8 | TGFBETA1 | IL6 | 89.7 |
| BDNF | M2PK | IL8 | TGFBETA1 | IL13 | 89.6 |
| BDNF | M2PK | IL8 | TGFBETA1 | IGFII | 89.6 |
| BDNF | M2PK | IL8 | TGFBETA1 | MIP1B | 88.5 |
| BDNF | M2PK | IL8 | MMP1 | MIP1B | 88.5 |
| BDNF | M2PK | IL8 | MAC2BP | MIP1B | 88.5 |

TABLE 31
Four biomarker combinations which differentiate colorectal
cancer subjects (female) from controls at 95% specificity.

| | | | | Cross-validated |
|---|---|---|---|---|
| BM1 | BM2 | BM3 | BM4 | sensitivity (%) |
| BDNF | M2PK | IL8 | MMP1 | 89.7 |
| BDNF | M2PK | IL8 | LIPOCALIN | 88.5 |
| BDNF | M2PK | IL8 | IL6 | 88.5 |
| BDNF | M2PK | IL8 | MIP1B | 87.2 |
| BDNF | M2PK | IL8 | MAC2BP | 87.2 |
| BDNF | M2PK | IL8 | TGFBETA1 | 85.9 |
| BDNF | M2PK | IL8 | IL13 | 85.7 |
| BDNF | M2PK | IL8 | IGFII | 85.7 |
| BDNF | M2PK | IL8 | TNFALPHA | 84.6 |
| BDNF | M2PK | TGFBETA1 | MMP1 | 84.6 |

TABLE 32
Three biomarker combinations which differentiate colorectal
cancer subjects (female) from controls at 95% specificity.

| | | | Cross Validated |
|---|---|---|---|
| BM1 | BM2 | BM3 | Sensitivity (%) |
| BDNF | M2PK | IL8 | 85.9 |
| BDNF | M2PK | MAC2BP | 79.5 |
| BDNF | M2PK | MMP1 | 78.2 |
| BDNF | M2PK | LIPOCALIN | 78.2 |
| BDNF | M2PK | IL13 | 77.9 |
| BDNF | M2PK | MIP1B | 76.9 |
| BDNF | M2PK | IL6 | 76.9 |
| BDNF | M2PK | TIMP1 | 76.6 |
| BDNF | M2PK | DKK3 | 75.6 |
| BDNF | M2PK | TNFALPHA | 74.4 |

TABLE 33
Two biomarker combinations which differentiate colorectal
cancer subjects (female) from controls at 95% specificity.

| | | Cross-validated |
|---|---|---|
| BM1 | BM2 | sensitivity (%) |
| BDNF | M2PK | 75.6 |

TABLE 34
Ten biomarker combinations plus age which differentiate colorectal
cancer subjects (female) from controls at 95% specificity.

| | | | | | | | | | | Cross- |
|---|---|---|---|---|---|---|---|---|---|---|
| | | | | | | | | | | validated |
| | | | | | | | | | | sensitivity |
| BM1 | BM2 | BM3 | BM4 | BM5 | BM6 | BM7 | BM8 | BM9 | BM10 | (%) |
| BDNF | M2PK | LIPOCALIN | IL8 | MMP1 | DKK3 | IGFBP2 | IGFII | IL6 | MAC2BP | 93.4 |
| BDNF | M2PK | LIPOCALIN | M65 | MMP1 | DKK3 | IGFBP2 | IGFII | IL6 | MAC2BP | 93.3 |
| BDNF | M2PK | LIPOCALIN | IL8 | MMP1 | DKK3 | IGFBP2 | IGFII | TNFALPHA | MIP1B | 92.1 |
| BDNF | M2PK | LIPOCALIN | IL8 | MMP1 | DKK3 | IGFBP2 | M30 | TIMP1 | TGFBETA1 | 92.1 |
| BDNF | M2PK | LIPOCALIN | IL8 | MMP1 | DKK3 | IGFBP2 | IGFII | IL6 | TGFBETA1 | 92.1 |
| BDNF | M2PK | M30 | M65 | MMP1 | DKK3 | IGFBP2 | MIP1B | IL6 | MAC2BP | 92 |
| BDNF | M2PK | LIPOCALIN | IL8 | MMP1 | DKK3 | IGFBP2 | IGFII | TNFALPHA | TIMP1 | 92 |
| BDNF | M2PK | LIPOCALIN | IL8 | MMP1 | DKK3 | IGFBP2 | M65 | TNFALPHA | MAC2BP | 92 |
| BDNF | M2PK | LIPOCALIN | M65 | MMP1 | DKK3 | IGFBP2 | TNFALPHA | IL6 | MAC2BP | 92 |
| BDNF | M2PK | LIPOCALIN | IL8 | MMP1 | DKK3 | IGFBP2 | IGFII | TNFALPHA | M65 | 92 |

TABLE 35
Nine biomarker combinations plus age which differentiate colorectal
cancer subjects (female) from controls at 95% specificity.

| | | | | | | | | | Cross- |
|---|---|---|---|---|---|---|---|---|---|
| | | | | | | | | | validated |
| | | | | | | | | | sensitivity |
| BM1 | BM2 | BM3 | BM4 | BM5 | BM6 | BM7 | BM8 | BM9 | (%) |
| BDNF | M2PK | LIPOCALIN | M65 | DKK3 | IGFBP2 | IL6 | MAC2BP | MMP1 | 94.7 |
| BDNF | M2PK | LIPOCALIN | TGFBETA1 | DKK3 | IGFBP2 | IL6 | MAC2BP | MMP1 | 93.5 |
| BDNF | M2PK | IL8 | LIPOCALIN | DKK3 | IGFBP2 | IL6 | IGFII | MMP1 | 93.4 |
| BDNF | M2PK | LIPOCALIN | M65 | DKK3 | IGFBP2 | IL6 | IL13 | MMP1 | 93.2 |
| BDNF | M2PK | LIPOCALIN | M65 | TGFBETA1 | MIP1B | IL6 | MAC2BP | MMP1 | 92.1 |
| BDNF | M2PK | LIPOCALIN | M65 | MIP1B | IGFII | IL6 | MAC2BP | MMP1 | 92.1 |
| BDNF | M2PK | IL8 | LIPOCALIN | DKK3 | IGFBP2 | IGFII | TNFALPHA | MMP1 | 92.1 |
| BDNF | M2PK | IL8 | LIPOCALIN | TGFBETA1 | IGFBP2 | IL6 | IGFII | MMP1 | 92.1 |
| BDNF | M2PK | IL8 | M65 | MIP1B | M30 | IL6 | TNFALPHA | IL13 | 92 |
| BDNF | M2PK | IL8 | M65 | MIP1B | M30 | IL6 | IGFII | IL13 | 92 |

TABLE 36
Eight biomarker combinations plus age which differentiate colorectal
cancer subjects (female) from controls at 95% specificity.

| | | | | | | | | Cross- |
|---|---|---|---|---|---|---|---|---|
| | | | | | | | | validated |
| | | | | | | | | sensitivity |
| BM1 | BM2 | BM3 | BM4 | BM5 | BM6 | BM7 | BM8 | (%) |
| BDNF | M2PK | M65 | IGFBP2 | MMP1 | IL6 | LIPOCALIN | DKK3 | 94.7 |
| BDNF | M2PK | IL8 | IGFBP2 | IGFII | IL6 | LIPOCALIN | MMP1 | 93.4 |
| BDNF | M2PK | M65 | MAC2BP | MMP1 | IL6 | LIPOCALIN | MIP1B | 92.1 |
| BDNF | M2PK | IL8 | IGFBP2 | IGFII | MMP1 | LIPOCALIN | MAC2BP | 92.1 |
| BDNF | M2PK | IL8 | M65 | M30 | IL6 | IL13 | MIP1B | 92 |
| BDNF | M2PK | IL8 | IGFBP2 | MMP1 | TGFBETA1 | LIPOCALIN | MAC2BP | 90.9 |
| BDNF | M2PK | IL8 | IGFBP2 | MAC2BP | IL6 | LIPOCALIN | TGFBETA1 | 90.9 |
| BDNF | M2PK | IL8 | IGFBP2 | MAC2BP | IL6 | TGFBETA1 | DKK3 | 90.9 |
| BDNF | M2PK | IL8 | M65 | MMP1 | M30 | TGFBETA1 | MIP1B | 90.8 |
| BDNF | M2PK | MAC2BP | M65 | MMP1 | MIP1B | LIPOCALIN | DKK3 | 90.8 |

TABLE 37
Seven biomarker combinations plus age which differentiate colorectal
cancer subjects (female) from controls at 95% specificity.

| | | | | | | | Cross- |
|---|---|---|---|---|---|---|---|
| | | | | | | | validated |
| | | | | | | | sensitivity |
| BM1 | BM2 | BM3 | BM4 | BM5 | BM6 | BM7 | (%) |
| BDNF | M2PK | IL8 | MMP1 | LIPOCALIN | IGFBP2 | TGFBETA1 | 92.2 |
| BDNF | M2PK | IL8 | MMP1 | LIPOCALIN | IGFBP2 | IGFII | 92.1 |
| BDNF | M2PK | IL8 | M30 | M65 | IL13 | IL6 | 92 |
| BDNF | M2PK | IL8 | MMP1 | LIPOCALIN | IGFBP2 | MAC2BP | 90.9 |
| BDNF | M2PK | IL8 | M30 | M65 | IL13 | TNFALPHA | 90.7 |
| BDNF | M2PK | IL8 | M30 | M65 | IL13 | IGFII | 90.7 |
| BDNF | M2PK | DKK3 | MMP1 | M65 | IGFBP2 | MAC2BP | 90.7 |
| BDNF | M2PK | IL8 | MMP1 | IL6 | MAC2BP | TGFBETA1 | 89.7 |
| BDNF | M2PK | IL8 | M30 | MIP1B | IL13 | TNFALPHA | 89.6 |
| BDNF | M2PK | IL8 | M30 | MIP1B | IL13 | TGFBETA1 | 89.6 |

TABLE 38
Six biomarker combinations plus age which differentiate colorectal
cancer subjects (female) from controls at 95% specificity.

| | | | | | | Cross- |
|---|---|---|---|---|---|---|
| | | | | | | validated |
| | | | | | | sensitivity |
| BM1 | BM2 | BM3 | BM4 | BM5 | BM6 | (%) |
| BDNF | M2PK | IL8 | MMP1 | LIPOCALIN | IGFBP2 | 92.2 |
| BDNF | M2PK | IL8 | M30 | IL13 | MIP1B | 89.6 |
| BDNF | M2PK | IL8 | M30 | IL13 | TGFBETA1 | 89.6 |
| BDNF | M2PK | IL8 | MMP1 | LIPOCALIN | TIMP1 | 89.6 |
| BDNF | M2PK | IL8 | M30 | IL13 | IGFII | 89.5 |
| BDNF | M2PK | IL8 | M30 | IL13 | M65 | 89.3 |
| BDNF | M2PK | MAC2BP | MMP1 | TGFBETA1 | MIP1B | 88.5 |
| BDNF | M2PK | IL8 | MMP1 | TGFBETA1 | TNFALPHA | 88.5 |
| BDNF | M2PK | IL8 | MAC2BP | TGFBETA1 | TNFALPHA | 88.5 |
| BDNF | M2PK | IL8 | MMP1 | LIPOCALIN | TNFALPHA | 88.5 |

TABLE 39
Five biomarker combinations plus age which differentiate colorectal
cancer subjects (female) from controls at 95% specificity.

| | | | | | Cross-Validated |
|---|---|---|---|---|---|
| BM1 | BM2 | BM3 | BM4 | BM5 | Sensitivity (%) |
| BDNF | M2PK | IL6 | IL8 | MIP1B | 88.5 |
| BDNF | M2PK | IL6 | IL8 | MMP1 | 88.5 |
| BDNF | M2PK | LIPOCALIN | IL8 | MMP1 | 88.5 |
| BDNF | M2PK | MAC2BP | IL8 | MMP1 | 87.2 |
| BDNF | M2PK | TGFBETA1 | IL8 | MIP1B | 87.2 |
| BDNF | M2PK | TNFALPHA | IL8 | MMP1 | 87.2 |
| BDNF | M2PK | TGFBETA1 | IL8 | MMP1 | 87.2 |
| BDNF | M2PK | TGFBETA1 | IL8 | MAC2BP | 87.2 |
| BDNF | M2PK | TGFBETA1 | MIP1B | MMP1 | 87.2 |
| BDNF | M2PK | IL13 | IL8 | MIP1B | 87 |

TABLE 40
Four biomarker combinations plus age which differentiate
colorectal cancer subjects (female) from controls at
95% specificity.

| | | | | Cross-Validated |
|---|---|---|---|---|
| BM1 | BM2 | BM3 | BM4 | Sensitivity (%) |
| BDNF | M2PK | IL8 | MMP1 | 88.5 |
| BDNF | M2PK | IL8 | MIP1B | 85.9 |
| BDNF | M2PK | IL8 | TGFBETA1 | 85.9 |
| BDNF | M2PK | IL8 | MAC2BP | 84.6 |
| BDNF | M2PK | IL8 | M30 | 84.6 |
| BDNF | M2PK | IL8 | IL13 | 84.4 |
| BDNF | M2PK | IL8 | LIPOCALIN | 83.3 |
| BDNF | M2PK | TGFBETA1 | MMP1 | 83.3 |
| BDNF | M2PK | MIP1B | MMP1 | 82.1 |
| BDNF | M2PK | MAC2BP | MMP1 | 82.1 |

TABLE 41
Three biomarker combinations plus age which
differentiate colorectal cancer subjects
(female) from controls at 95% specificity.

| BM1 | BM2 | BM3 | Cross-Validated Sensitivity (%) |
|---|---|---|---|
| BDNF | M2PK | IL8 | 83.3 |
| BDNF | M2PK | MAC2BP | 79.5 |
| BDNF | M2PK | MMP1 | 79.5 |
| BDNF | M2PK | MIP1B | 76.9 |
| BDNF | M2PK | IL6 | 76.9 |
| BDNF | M2PK | M30 | 75.6 |
| BDNF | M2PK | IL13 | 75.3 |
| BDNF | M2PK | TIMP1 | 75.3 |
| BDNF | M2PK | LIPOCALIN | 74.4 |
| BDNF | M2PK | DKK3 | 74.4 |

TABLE 42
Two biomarker combinations plus age which
differentiate colorectal cancer subjects
(female) from controls at 95% specificity.

| BM1 | BM2 | Cross-validated sensitivity (%) |
|---|---|---|
| BDNF | M2PK | 78.2 |

Considering tables 25-33 and 34-42 above generally reveals that whether or not age is included in the algorithm, the top performing sets of biomarkers, developed from female-only participants achieved higher sensitivity at 95% specificity when panels contained progressively higher numbers of biomarkers. For biomarker panels of six to nine, the observed maximum sensitivity achieved with female only data increases marginally when age is included in the model.

Comparing Tables 7-15 (genders combined, biomarkers only) with Tables 25-33 (females, biomarkers only), it was surprising to observe that cross-validated models with significantly higher sensitivity at 95% specificity were identified when modelled on data from females alone than when modelled on data from both genders combined. Differentials as high as 10% were observed in all biomarker panel size classes and were observed whether or not age was included in the modelling (compare Tables 16-24 with Tables 34-42 for modelling where age is included).

As for models built on combined male and female data, for female-only developed models, the biomarker compositions for top performing panel in each biomarker panel differed somewhat between models that included and did not include age (exceptions were three and four biomarker panel size classes where the top model in each class had the same biomarker composition in the presence and absence of age). However, biomarkers that were prominent in the top models for each biomarker numerical class in the absence of age were conserved in the equivalent models where age was included. In particular, BDNF and M2PK were present in all models. IL8 and MMP1 were prominent across many classes. While IGFBP2 and LIPOCALIN did not appear in any top model in any biomarker panel derived from female-only data in the absence of age, when age was included in the analysis, both of these markers were prominent in six to ten biomarker panels.

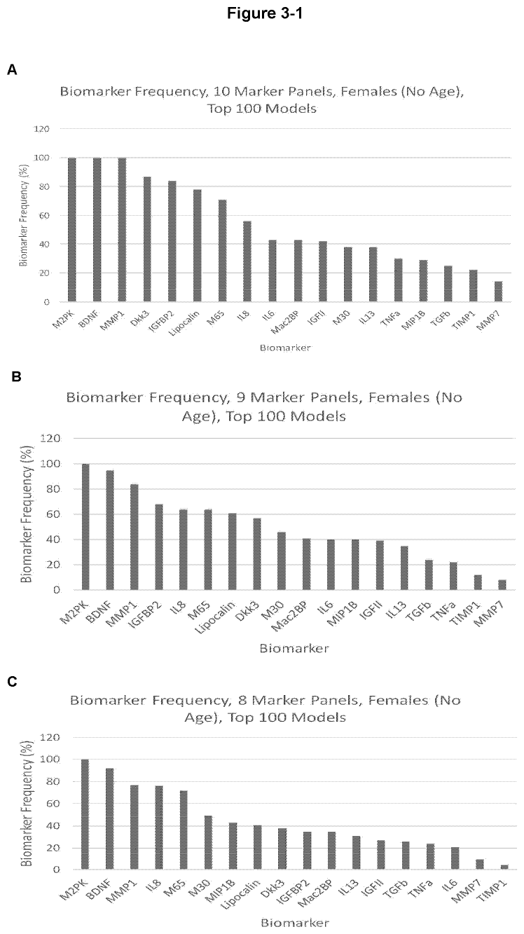

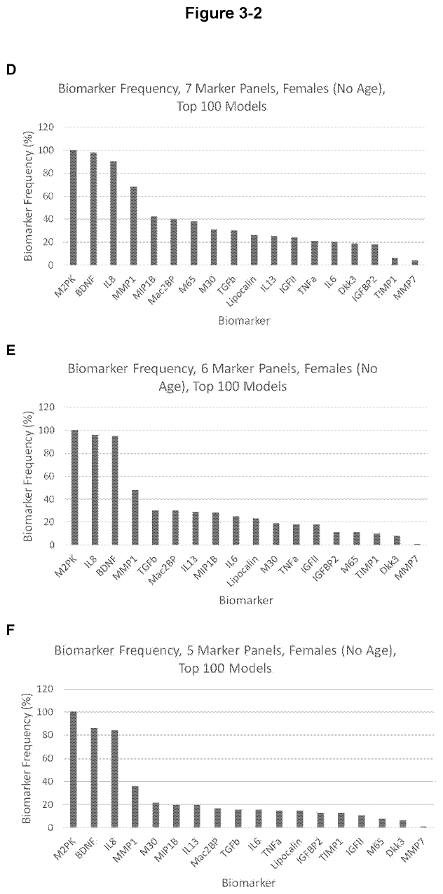

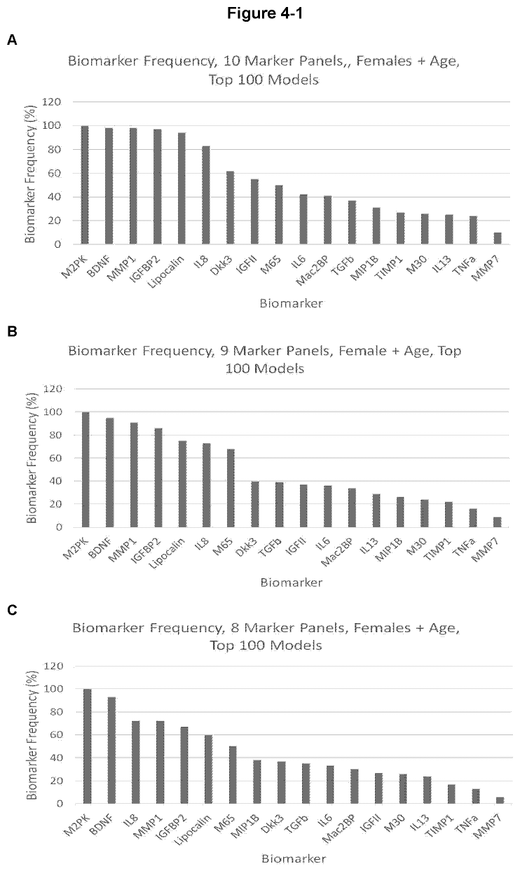

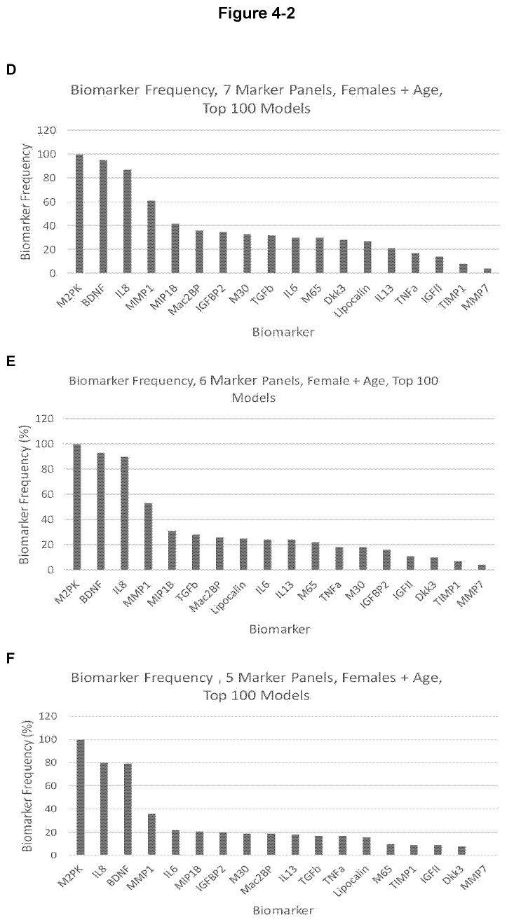

FIGS. 3-1 and 3-2 show graphs depicting the frequency with which individual biomarkers (when age is not considered) are represented in the top 100 models (or top models producing a sensitivity of ≥75% at 95% specificity) developed on female-only data for biomarker panels containing five to ten biomarkers. FIGS. 4-1 and 4-2 show graphs depicting the frequency with which individual biomarkers (when age is considered) are represented in the top 100 models (or top models producing a sensitivity of ≥75% at 95% specificity) developed on female-only data for biomarker panels containing five to ten biomarkers.

For models based on female data only, M2PK was present in all high performing models in all numerical classes irrespective of whether age was included or not. BDNF was present in ≥80% of high performing biomarker panels in five to ten biomarker combinations regardless of whether age was included in the modelling. In the three and four biomarker panels, BDNF remained the second most frequent biomarker irrespective of inclusion of age. For female-only biomarker combinations of eight to ten biomarkers, other prominent biomarkers (present in >50% of this broader selection of high performing models) independent of inclusion of age included, MMP1, M65 and IL8. When an age term is included, Lipocalin and IGFBP2 were also represented in 50% of these high-performing panels. For four to seven biomarker panels, IL8 remained prominent. MMP1 was also amongst the more commonly included biomarkers although its frequency dropped below 50% in panels of three to five biomarkers. IL6 was represented in ≥20% of high performing panels in three and four biomarker panels irrespective of whether age was included in the modelling.

Examples of the top ten biomarker combinations for panels of two to ten biomarkers developed on male only data are shown in Tables 43-51. Examples of the top ten biomarker combinations for panels of two to ten biomarkers developed on male only data when age was also considered are shown in Tables 52-60.

TABLE 43
Ten biomarker combinations which include BDNF and differentiate colorectal
cancer subjects (male) from controls at 95% specificity.

| | | | | | | | | | | Cross- |
|---|---|---|---|---|---|---|---|---|---|---|
| | | | | | | | | | | validated |
| | | | | | | | | | | sensitivity |
| BM1 | BM2 | BM3 | BM4 | BM5 | BM6 | BM7 | BM8 | BM9 | BM10 | (%) |
| BDNF | M2PK | DKK3 | TNFALPHA | IL8 | MIP1B | TIMP1 | TGFBETA1 | LIPOCALIN | MAC2BP | 84 |
| BDNF | M2PK | DKK3 | TNFALPHA | IL8 | MIP1B | TIMP1 | TGFBETA1 | LIPOCALIN | MMP7 | 82.9 |
| BDNF | M2PK | DKK3 | TNFALPHA | MMP1 | MIP1B | TIMP1 | IGFBP2 | LIPOCALIN | MMP7 | 82.1 |
| BDNF | M2PK | DKK3 | TNFALPHA | IL8 | MMP1 | M30 | IGFBP2 | M65 | IGFII | 82.1 |
| BDNF | M2PK | DKK3 | TNFALPHA | IL8 | MMP1 | IGFII | IGFBP2 | LIPOCALIN | M65 | 82.1 |
| BDNF | M2PK | DKK3 | TNFALPHA | IL8 | MMP1 | IGFII | IGFBP2 | M65 | MMP7 | 81.9 |
| BDNF | M2PK | DKK3 | TNFALPHA | IGFII | MMP1 | M65 | TGFBETA1 | IGFBP2 | MMP7 | 81.7 |
| BDNF | M2PK | DKK3 | TNFALPHA | MMP1 | MIP1B | TIMP1 | IGFBP2 | LIPOCALIN | IGFII | 81.3 |
| BDNF | M2PK | DKK3 | TNFALPHA | IL8 | MMP1 | IL13 | IGFBP2 | IGFII | MAC2BP | 81.3 |
| BDNF | M2PK | DKK3 | TNFALPHA | MMP1 | MIP1B | TIMP1 | IGFBP2 | IL13 | MMP7 | 81.1 |

TABLE 44
Nine biomarker combinations which include BDNF and differentiate colorectal
cancer subjects (male) from controls at 95% specificity.

| | | | | | | | | | Cross- |
|---|---|---|---|---|---|---|---|---|---|
| | | | | | | | | | validated |
| | | | | | | | | | sensitivity |
| BM1 | BM2 | BM3 | BM4 | BM5 | BM6 | BM7 | BM8 | BM9 | (%) |
| BDNF | M2PK | DKK3 | TNFALPHA | MIP1B | TIMP1 | IL8 | TGFBETA1 | LIPOCALIN | 84 |
| BDNF | M2PK | DKK3 | TNFALPHA | MIP1B | TIMP1 | IGFBP2 | MMP1 | LIPOCALIN | 83.2 |
| BDNF | M2PK | DKK3 | TNFALPHA | MIP1B | TIMP1 | IL8 | TGFBETA1 | MAC2BP | 83 |
| BDNF | M2PK | DKK3 | TNFALPHA | IGFII | IGFBP2 | IL8 | MMP1 | M65 | 83 |
| BDNF | M2PK | DKK3 | TNFALPHA | MIP1B | IGFBP2 | TIMP1 | IL6 | M30 | 82.2 |
| BDNF | M2PK | DKK3 | TNFALPHA | MIP1B | IGFBP2 | IL8 | MMP1 | IGFII | 82.2 |
| BDNF | M2PK | DKK3 | TNFALPHA | M30 | IGFBP2 | TIMP1 | IL6 | LIPOCALIN | 82.2 |
| BDNF | M2PK | DKK3 | TNFALPHA | MIP1B | MMP7 | TIMP1 | IL8 | LIPOCALIN | 82.1 |
| BDNF | M2PK | DKK3 | TNFALPHA | MIP1B | IGFBP2 | MMP7 | MMP1 | IGFII | 82.1 |
| BDNF | M2PK | DKK3 | TNFALPHA | MMP7 | IGFBP2 | IGFII | LIPOCALIN | IL13 | 82.1 |

TABLE 45
Eight biomarker combinations which include BDNF and differentiate
colorectal cancer subjects (male) from controls at 95% specificity.

| | | | | | | | | Cross- |
|---|---|---|---|---|---|---|---|---|
| | | | | | | | | validated |
| | | | | | | | | sensitivity |
| BM1 | BM2 | BM3 | BM4 | BM5 | BM6 | BM7 | BM8 | (%) |
| BDNF | M2PK | DKK3 | TNFALPHA | MIP1B | TIMP1 | IL8 | TGFBETA1 | 84 |
| BDNF | M2PK | DKK3 | TNFALPHA | MIP1B | MMP1 | MMP7 | IGFBP2 | 84 |
| BDNF | M2PK | DKK3 | TNFALPHA | MIP1B | TIMP1 | IL8 | LIPOCALIN | 82.2 |
| BDNF | M2PK | DKK3 | TNFALPHA | MIP1B | MMP1 | IGFII | IGFBP2 | 82.2 |
| BDNF | M2PK | DKK3 | TNFALPHA | MIP1B | TIMP1 | IL8 | MMP7 | 82.1 |
| BDNF | M2PK | DKK3 | TNFALPHA | MMP1 | TGFBETA1 | TIMP1 | IGFBP2 | 82.1 |
| BDNF | M2PK | DKK3 | TNFALPHA | IGFBP2 | M30 | IL13 | MMP7 | 82.1 |
| BDNF | M2PK | DKK3 | TNFALPHA | IGFBP2 | IL13 | IGFII | MMP7 | 82.1 |
| BDNF | M2PK | DKK3 | TNFALPHA | LIPOCALIN | TIMP1 | IL8 | MMP7 | 82.1 |
| BDNF | M2PK | DKK3 | TNFALPHA | IGFBP2 | IGFII | MMP1 | MMP7 | 82.1 |

TABLE 46
Seven biomarker combinations which include BDNF and differentiate
colorectal cancer subjects (male) from controls at 95% specificity.

| | | | | | | | Cross- |
|---|---|---|---|---|---|---|---|
| | | | | | | | validated |
| | | | | | | | sensitivity |
| BM1 | BM2 | BM3 | BM4 | BM5 | BM6 | BM7 | (%) |
| BDNF | M2PK | DKK3 | TNFALPHA | TIMP1 | MIP1B | IL8 | 82.2 |
| BDNF | M2PK | DKK3 | TNFALPHA | TIMP1 | MIP1B | IGFBP2 | 82.2 |
| BDNF | M2PK | DKK3 | TNFALPHA | TIMP1 | LIPOCALIN | IL8 | 82.2 |
| BDNF | M2PK | DKK3 | TNFALPHA | M65 | MIP1B | IGFBP2 | 82.1 |
| BDNF | M2PK | DKK3 | TNFALPHA | TIMP1 | MMP7 | IL8 | 82.1 |
| BDNF | M2PK | DKK3 | TNFALPHA | MMP1 | MMP7 | IGFBP2 | 82.1 |
| BDNF | M2PK | DKK3 | TNFALPHA | IGFII | MMP7 | IGFBP2 | 82.1 |
| BDNF | M2PK | DKK3 | TNFALPHA | M65 | M30 | IGFBP2 | 82.1 |
| BDNF | M2PK | DKK3 | TNFALPHA | IGFII | M30 | IGFBP2 | 81.3 |
| BDNF | M2PK | DKK3 | TNFALPHA | MIP1B | MMP7 | IGFBP2 | 81.1 |

TABLE 47
Six biomarker combinations which include BDNF and differentiate colorectal
cancer subjects (male) from controls at 95% specificity.

| | | | | | | Cross- |
|---|---|---|---|---|---|---|
| | | | | | | validated |
| | | | | | | sensitivity |
| BM1 | BM2 | BM3 | BM4 | BM5 | BM6 | (%) |
| BDNF | M2PK | DKK3 | TNFALPHA | TIMP1 | IL8 | 82.2 |
| BDNF | M2PK | DKK3 | TNFALPHA | IGFBP2 | MMP1 | 81.3 |
| BDNF | M2PK | DKK3 | TNFALPHA | IGFBP2 | M30 | 81.3 |
| BDNF | M2PK | DKK3 | TNFALPHA | MIP1B | M65 | 81.1 |
| BDNF | M2PK | DKK3 | TNFALPHA | IGFBP2 | MMP7 | 81.1 |
| BDNF | M2PK | DKK3 | TNFALPHA | IGFBP2 | M65 | 81.1 |
| BDNF | M2PK | DKK3 | TNFALPHA | IGFBP2 | LIPOCALIN | 80.4 |
| BDNF | M2PK | DKK3 | TNFALPHA | IGFII | IL8 | 80.4 |
| BDNF | M2PK | DKK3 | TNFALPHA | M65 | M30 | 80.2 |
| BDNF | M2PK | DKK3 | TNFALPHA | IGFBP2 | IL13 | 79.4 |

TABLE 48
Five biomarker combinations which include BDNF and differentiate
colorectal cancer subjects (male) from controls at 95% specificity.

| BM1 | BM2 | BM3 | BM4 | BM5 | Cross-validated sensitivity (%) |
|---|---|---|---|---|---|
| BDNF | M2PK | DKK3 | IGFBP2 | TNFALPHA | 80.4 |
| BDNF | M2PK | DKK3 | LIPOCALIN | TIMP1 | 78.5 |
| BDNF | M2PK | DKK3 | M65 | TNFALPHA | 78.3 |
| BDNF | M2PK | DKK3 | IGFBP2 | TIMP1 | 77.6 |
| BDNF | M2PK | DKK3 | IL8 | IGFII | 77.6 |
| BDNF | M2PK | IL13 | IL8 | TNFALPHA | 76.6 |
| BDNF | M2PK | DKK3 | MAC2BP | TIMP1 | 76.6 |
| BDNF | M2PK | DKK3 | M65 | TIMP1 | 76.4 |
| BDNF | M2PK | DKK3 | MIP1B | TNFALPHA | 75.7 |
| BDNF | M2PK | DKK3 | MIP1B | TIMP1 | 75.7 |

TABLE 49
Four biomarker combinations which include BDNF and differentiate
colorectal cancer subjects (male) from controls at 95% specificity.

| BM1 | BM2 | BM3 | BM4 | Cross-validated sensitivity (%) |
|---|---|---|---|---|
| BDNF | M2PK | DKK3 | TIMP1 | 76.6 |
| BDNF | M2PK | IL8 | TNFALPHA | 73.8 |
| BDNF | M2PK | IGFBP2 | TNFALPHA | 72 |
| BDNF | M2PK | IL8 | IGFBP2 | 72 |
| BDNF | M2PK | DKK3 | TNFALPHA | 71 |
| BDNF | M2PK | IL8 | MMP7 | 70.8 |
| BDNF | M2PK | DKK3 | M65 | 70.8 |
| BDNF | M2PK | DKK3 | IL8 | 70.1 |
| BDNF | M2PK | DKK3 | IGFBP2 | 70.1 |
| BDNF | M2PK | MIP1B | TIMP1 | 69.2 |

TABLE 50
Three biomarker combinations which include BDNF and
differentiate colorectal cancer subjects (male) from controls
at 95% specificity.

| BM1 | BM2 | BM3 | Cross-validated sensitivity (%) |
|---|---|---|---|
| BDNF | M2PK | TNFALPHA | 69.2 |
| BDNF | M2PK | TIMP1 | 69.2 |
| BDNF | M2PK | IL8 | 68.2 |
| BDNF | M2PK | TGFBETA1 | 62.3 |
| BDNF | M2PK | MMP7 | 62.3 |
| BDNF | M2PK | IL13 | 58.9 |
| BDNF | M2PK | IGFII | 58.9 |
| BDNF | M2PK | IGFBP2 | 58.9 |
| BDNF | M2PK | MAC2BP | 57.9 |
| BDNF | M2PK | LIPOCALIN | 57.9 |

TABLE 51
Two biomarker combinations which include BDNF
and differentiate colorectal cancer subjects
(male) from controls at 95% specificity.

| BM1 | BM2 | Cross-validated sensitivity (%) |
|---|---|---|
| BDNF | M2PK | 52.3 |

TABLE 52
Ten biomarker combinations plus age which include BDNF and differentiate
colorectal cancer subjects (male) from controls at 95% specificity.

| | | | | | | | | | | Cross- |
|---|---|---|---|---|---|---|---|---|---|---|
| | | | | | | | | | | validated |
| | | | | | | | | | | sensitivity |
| BM1 | BM2 | BM3 | BM4 | BM5 | BM6 | BM7 | BM8 | BM9 | BM10 | (%) |
| BDNF | M2PK | DKK3 | TNFALPHA | IGFBP2 | IL8 | M30 | MAC2BP | MMP1 | TIMP1 | 82.2 |
| BDNF | M2PK | DKK3 | TNFALPHA | IGFBP2 | IL8 | M30 | MAC2BP | MMP1 | IL6 | 82.2 |
| BDNF | M2PK | DKK3 | TNFALPHA | IGFBP2 | IL8 | MIP1B | MAC2BP | MMP1 | M65 | 82.1 |
| BDNF | M2PK | DKK3 | TNFALPHA | IGFBP2 | IL8 | M30 | IGFII | MMP1 | M65 | 82.1 |
| BDNF | M2PK | DKK3 | TNFALPHA | IGFBP2 | IL8 | MIP1B | MAC2BP | MMP1 | TIMP1 | 81.3 |
| BDNF | M2PK | DKK3 | TNFALPHA | IGFBP2 | IL8 | M30 | MAC2BP | MMP1 | MIP1B | 81.3 |
| BDNF | M2PK | DKK3 | TNFALPHA | IGFBP2 | IL8 | M30 | MIP1B | MMP1 | IGFII | 81.3 |
| BDNF | M2PK | DKK3 | TNFALPHA | IL13 | IL8 | IGFII | MAC2BP | M65 | IL6 | 81.1 |
| BDNF | M2PK | DKK3 | TNFALPHA | IGFBP2 | MMP7 | M30 | MIP1B | MMP1 | M65 | 81 |
| BDNF | M2PK | DKK3 | TNFALPHA | IGFBP2 | TGFBETA1 | M30 | IGFII | MMP1 | M65 | 81 |

TABLE 53
Nine biomarker combinations plus age which include BDNF and differentiate
colorectal cancer subjects (male) from controls at 95% specificity.

| | | | | | | | | | Cross- |
|---|---|---|---|---|---|---|---|---|---|
| | | | | | | | | | validated |
| | | | | | | | | | sensitivity |
| BM1 | BM2 | BM3 | BM4 | BM5 | BM6 | BM7 | BM8 | BM9 | (%) |
| BDNF | M2PK | DKK3 | TNFALPHA | IGFBP2 | MMP1 | M30 | MMP7 | MIP1B | 82.1 |
| BDNF | M2PK | DKK3 | TNFALPHA | IGFBP2 | MMP1 | IGFII | MMP7 | MIP1B | 82.1 |
| BDNF | M2PK | DKK3 | TNFALPHA | IGFBP2 | MMP1 | IGFII | IL13 | IL8 | 81.3 |
| BDNF | M2PK | DKK3 | TNFALPHA | IGFBP2 | MMP1 | M30 | IGFII | IL8 | 81.3 |
| BDNF | M2PK | DKK3 | TNFALPHA | IGFBP2 | IL13 | IGFII | MMP7 | MIP1B | 81.1 |
| BDNF | M2PK | DKK3 | TNFALPHA | TIMP1 | MMP1 | M30 | TGFBETA1 | MIP1B | 81.1 |
| BDNF | M2PK | DKK3 | TNFALPHA | TIMP1 | LIPOCALIN | IL8 | MMP7 | MIP1B | 81.1 |
| BDNF | M2PK | DKK3 | TNFALPHA | M65 | IL13 | IGFII | IL8 | IL6 | 81.1 |
| BDNF | M2PK | DKK3 | TNFALPHA | IGFBP2 | MMP1 | MAC2BP | IL8 | MIP1B | 80.4 |
| BDNF | M2PK | DKK3 | TNFALPHA | IGFBP2 | MMP1 | M30 | IL8 | TIMP1 | 80.4 |

TABLE 54
Eight biomarker combinations plus age which include BDNF and differentiate
colorectal cancer subjects (male) from controls at 95% specificity.

| | | | | | | | | Cross- |
|---|---|---|---|---|---|---|---|---|
| | | | | | | | | validated |
| | | | | | | | | sensitivity |
| BM1 | BM2 | BM3 | BM4 | BM5 | BM6 | BM7 | BM8 | (%) |
| BDNF | M2PK | DKK3 | TNFALPHA | TIMP1 | MIP1B | IL8 | LIPOCALIN | 82.2 |
| BDNF | M2PK | DKK3 | MMP1 | TIMP1 | IGFII | IL8 | IGFBP2 | 82.2 |
| BDNF | M2PK | DKK3 | TNFALPHA | TIMP1 | MIP1B | IL13 | M30 | 81.1 |
| BDNF | M2PK | DKK3 | TNFALPHA | TIMP1 | MIP1B | MMP1 | TGFBETA1 | 81.1 |
| BDNF | M2PK | DKK3 | TNFALPHA | TIMP1 | MIP1B | IL8 | MMP7 | 81.1 |
| BDNF | M2PK | DKK3 | TNFALPHA | MMP1 | MIP1B | MMP7 | IGFBP2 | 81.1 |
| BDNF | M2PK | DKK3 | TNFALPHA | M65 | MIP1B | M30 | LIPOCALIN | 81.1 |
| BDNF | M2PK | DKK3 | TNFALPHA | M65 | MIP1B | IL6 | IGFBP2 | 81.1 |
| BDNF | M2PK | DKK3 | TNFALPHA | MMP1 | MMP7 | IL8 | IGFBP2 | 81.1 |
| BDNF | M2PK | DKK3 | TNFALPHA | M65 | MMP7 | M30 | IGFBP2 | 81 |

TABLE 55
Seven biomarker combinations plus age which include BDNF and differentiate
colorectal cancer subjects (male) from controls at 95% specificity.

| | | | | | | | Cross- |
|---|---|---|---|---|---|---|---|
| | | | | | | | validated |
| | | | | | | | sensitivity |
| BM1 | BM2 | BM3 | BM4 | BM5 | BM6 | BM7 | (%) |
| BDNF | M2PK | DKK3 | TNFALPHA | MMP7 | IGFBP2 | M30 | 82.1 |
| BDNF | M2PK | DKK3 | TNFALPHA | IL8 | MIP1B | TIMP1 | 81.3 |
| BDNF | M2PK | DKK3 | TNFALPHA | IL8 | LIPOCALIN | TIMP1 | 81.3 |
| BDNF | M2PK | DKK3 | TNFALPHA | M65 | MIP1B | IL13 | 81.1 |
| BDNF | M2PK | DKK3 | TNFALPHA | M65 | MIP1B | M30 | 81.1 |
| BDNF | M2PK | DKK3 | TNFALPHA | MMP1 | TGFBETA1 | TIMP1 | 81.1 |
| BDNF | M2PK | DKK3 | TNFALPHA | MMP7 | IL8 | TIMP1 | 81.1 |
| BDNF | M2PK | DKK3 | TNFALPHA | MMP7 | IGFBP2 | M65 | 81 |
| BDNF | M2PK | DKK3 | IGFII | IL13 | LIPOCALIN | TIMP1 | 80.4 |
| BDNF | M2PK | DKK3 | TNFALPHA | IL8 | IGFBP2 | MMP1 | 80.4 |

TABLE 56
Six biomarker combinations plus age which include BDNF and differentiate
colorectal cancer subjects (male) from controls at 95% specificity.

| | | | | | | Cross- |
|---|---|---|---|---|---|---|
| | | | | | | validated |
| | | | | | | sensitivity |
| BM1 | BM2 | BM3 | BM4 | BM5 | BM6 | (%) |
| BDNF | M2PK | DKK3 | TNFALPHA | TIMP1 | IL8 | 81.3 |
| BDNF | M2PK | DKK3 | TNFALPHA | IGFBP2 | IL8 | 81.3 |
| BDNF | M2PK | DKK3 | TNFALPHA | M65 | MIP1B | 81.1 |
| BDNF | M2PK | DKK3 | TNFALPHA | M65 | M30 | 80.2 |
| BDNF | M2PK | DKK3 | MMP7 | TIMP1 | M30 | 80.2 |
| BDNF | M2PK | DKK3 | TNFALPHA | IGFBP2 | M30 | 79.4 |
| BDNF | M2PK | DKK3 | MMP1 | TIMP1 | IL8 | 79.4 |
| BDNF | M2PK | DKK3 | IGFII | TIMP1 | LIPOCALIN | 79.4 |
| BDNF | M2PK | DKK3 | TNFALPHA | IGFBP2 | MMP7 | 79.2 |
| BDNF | M2PK | DKK3 | TNFALPHA | IGFBP2 | M65 | 79.2 |

TABLE 57
Five biomarker combinations plus age which include BDNF
and differentiate colorectal cancer subjects (male) from
controls at 95% specificity.

| | | | | | Cross-validated |
|---|---|---|---|---|---|
| BM1 | BM2 | BM3 | BM4 | BM5 | sensitivity (%) |
| BDNF | M2PK | DKK3 | M65 | TNFALPHA | 79.2 |
| BDNF | M2PK | DKK3 | TIMP1 | MMP7 | 77.4 |
| BDNF | M2PK | DKK3 | M65 | MMP7 | 77.1 |
| BDNF | M2PK | IL8 | IL13 | TNFALPHA | 76.6 |
| BDNF | M2PK | DKK3 | IGFBP2 | TNFALPHA | 76.6 |
| BDNF | M2PK | DKK3 | TIMP1 | MAC2BP | 76.6 |
| BDNF | M2PK | DKK3 | TIMP1 | IGFII | 76.6 |
| BDNF | M2PK | DKK3 | MIP1B | TNFALPHA | 75.7 |
| BDNF | M2PK | DKK3 | TIMP1 | MIP1B | 75.7 |
| BDNF | M2PK | DKK3 | TIMP1 | IL13 | 75.7 |

TABLE 58
Four biomarker combinations plus age which include BDNF
and differentiate colorectal cancer subjects (male) from
controls at 95% specificity.

| BM1 | BM2 | BM3 | BM4 | Cross-validated sensitivity (%) |
|---|---|---|---|---|
| BDNF | M2PK | TNFALPHA | IGFBP2 | 75.7 |
| BDNF | M2PK | TIMP1 | DKK3 | 75.7 |
| BDNF | M2PK | TNFALPHA | IL8 | 74.8 |
| BDNF | M2PK | IL8 | DKK3 | 72 |
| BDNF | M2PK | TNFALPHA | DKK3 | 71 |
| BDNF | M2PK | IL8 | TIMP1 | 71 |
| BDNF | M2PK | IGFII | IGFBP2 | 71 |
| BDNF | M2PK | IL8 | MMP7 | 70.8 |
| BDNF | M2PK | M65 | DKK3 | 69.8 |
| BDNF | M2PK | TNFALPHA | MIP1B | 69.2 |

TABLE 59
Three biomarker combinations plus age which include
BDNF and differentiate colorectal cancer subjects (male)
from controls at 95% specificity.

| BM1 | BM2 | BM3 | Cross validated sensitivity (%) |
|---|---|---|---|
| BDNF | M2PK | IL8 | 68.2 |

TABLE 60
Two biomarker combinations plus age which include
BDNF and differentiate colorectal cancer subjects
(male) from controls at 95% specificity.

| BM1 | BM2 | Cross-validated sensitivity (%) |
|---|---|---|
| BDNF | M2PK | 57.9 |

Comparing Tables 7-15 (genders combined, biomarkers only) with Tables 43-51 (males, biomarkers only), the differences in performance of models developed on data from both genders combined and those developed on male-only data were minimal. This pattern was also observed where the modelling included age (Tables 16-24 compared with Tables 52-60). These results for males contrasted markedly from the analogous results obtained with models built on female-only data.

Consideration of Tables 43-51 and 52-60 revealed that, generally, whether or not age was included in the calculation, top performing sets of models developed on data from male-only subjects achieved higher sensitivity values at 95% specificity when the panels contain higher numbers of biomarkers. As observed for genders combined, there were overlaps in sensitivities achieved with top performing models between adjacent biomarker size classes ranging from two to ten obtained with male only data. No improvement in sensitivity was observed when an age term was included in modelling performed with male only data.

As for models built on combined male and female data, for male-only developed models, the biomarker compositions for the top performing panel in each biomarker numerical class differed somewhat between models that included and did not include age. The only exception was the five biomarker panel where the top model had the same biomarker composition in the presence and absence of age. However, there was still strong conservation of key biomarkers in the top performing models plus and minus age in each biomarker numerical class. In particular, BDNF and M2PK were present in all top models. DKK3 and TNFα were prominent across most biomarker combinations, while IL8 and TIMP1 were also fairly commonly represented. IGFBP2 was only represented in the biomarker-only top model of the five biomarker combination but was present in top models that include age of the four, seven, nine and ten biomarker combinations.

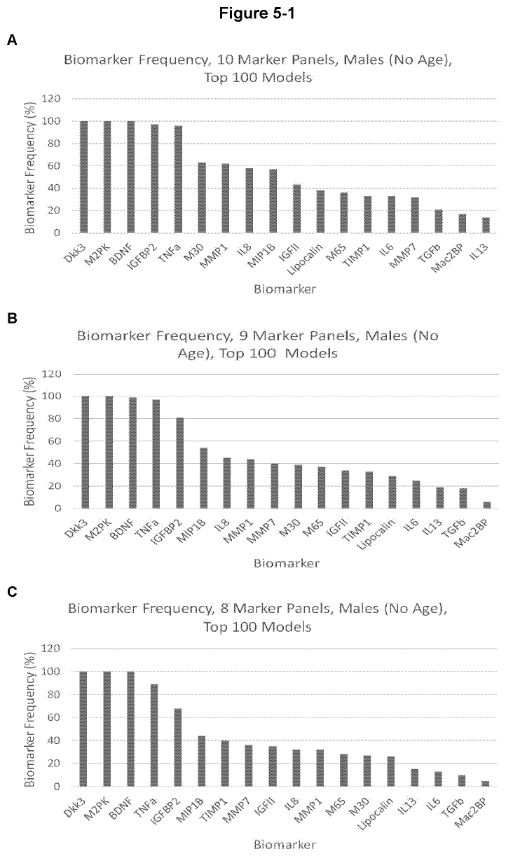

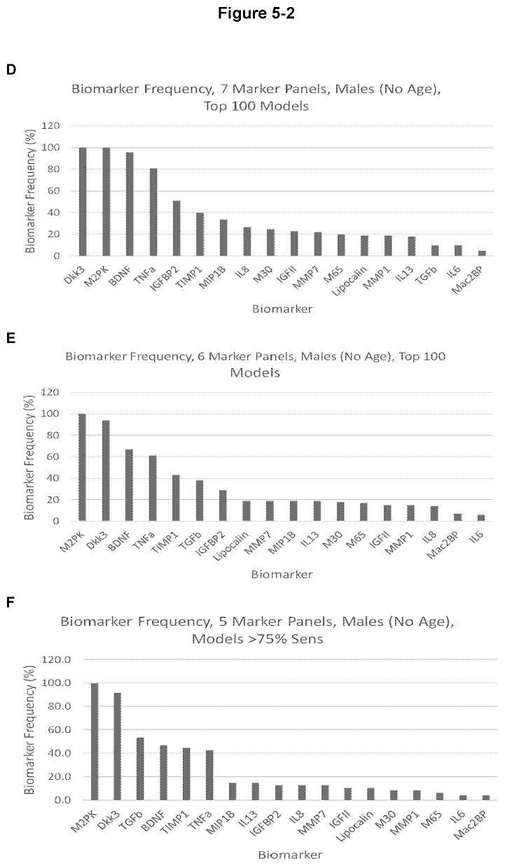

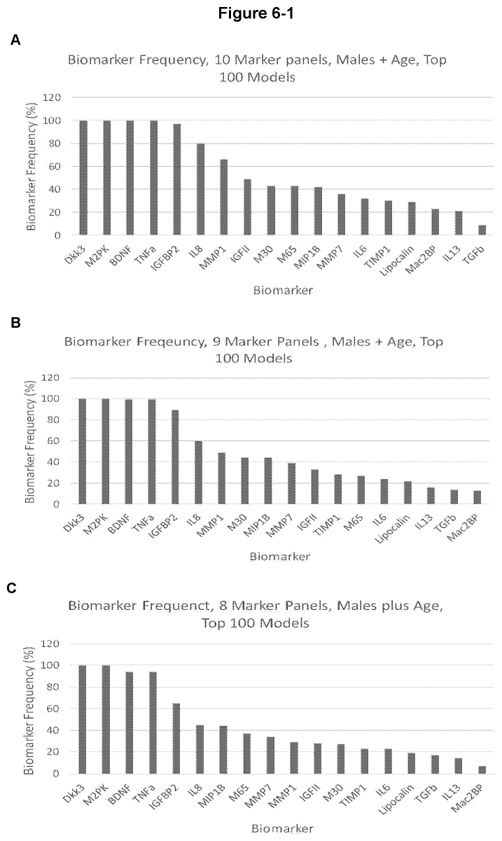

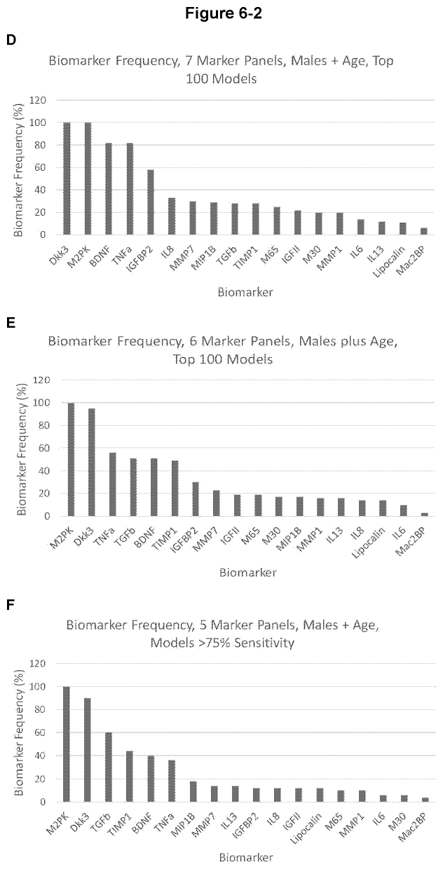

FIGS. 5-1 and 5-2 show graphs depicting the frequency with which individual biomarkers are represented in the top 100 models (or top models producing a sensitivity of ≥75% at 95% specificity) developed on male-only data for panels of five to ten biomarkers. FIGS. 6-1 and 6-2 show graphs depicting the frequency with which individual biomarkers (when age is considered) are represented in the top 100 models (or top models producing a sensitivity of ≥75% at 95% specificity) developed on male-only data for biomarker panels containing five to ten biomarkers.

For models based on male data only, M2PK was present in all high performing models in all biomarker numerical classes irrespective of whether age was included or not. Unlike in models built on analogous genders-combined and female only data sets, in the male-only models, DKK3 was the next most prevalent marker, being present in all panels of seven to ten biomarker combinations in this broader selection of high performing models and in over 80% of all models in four to six biomarker combinations, irrespective of whether they included age or not. BDNF was present in all models in eight to ten biomarker combinations, in over 90% of high performing seven biomarker panels and in over 50% of six biomarker combinations. Although its frequency dropped below 50% in four and five biomarker combinations, BDNF remained solidly within the second tier of biomarker frequencies. TNFα was prevalent in high performing six to ten biomarker combinations and was still a sound second tier marker in five biomarker combinations. IGFBP2 was also prevalent amongst high performing models of seven to ten biomarker combinations but its representation fell substantially in the three to six biomarker combinations. In contrast, TGFβ1, a minor contributor to top performing models in the seven to ten biomarker panels became a prominent contributor to the high performing four to six biomarker panels, becoming the third most frequently represented marker in the four and five biomarker panels.

For males there were no two-biomarker combinations (with or without age) that discriminated between case and control sera derived from males with a sensitivity>75% at a specificity of 95%. The best performing pairwise combination including BDNF (when age was not considered) for males was M2PK+BDNF—Sensitivity 52.3%. The best performing pairwise combination including BDNF (when age was included) for males was M2PK+BDNF—Sensitivity 57.9%.

### Example 5 Other High Performing Biomarker Combinations

The methods, uses and the like described herein may also use one or more or all of the biomarker combinations provided in Tables 61 to 66. In these tables Sens.=mean cross validated sensitivity.

TABLE 61
Top 20, 7 to 3 biomarker combinations which may be used to differentiate colorectal
cancer subjects (both genders, age not considered) from controls at 95% specificity.

| # | Sens. | Dkk3 | M2PK | TGFb | IGFBP2 | TIMP1 | BDNF | IL6 | IL8 | TNFa |
|---|---|---|---|---|---|---|---|---|---|---|
| 7 | 83.2% | DKK3 | M2PK | | IGFB2 | | BDNF | | | TNFa |
| 7 | 83.0% | DKK3 | M2PK | | IGFB2 | | BDNF | | | TNFa |
| 7 | 83.0% | DKK3 | M2PK | | | | BDNF | | IL8 | TNFa |
| 7 | 81.9% | DKK3 | M2PK | | IGFB2 | TIMP1 | BDNF | | | TNFa |
| 7 | 81.9% | DKK3 | M2PK | | IGFB2 | | BDNF | IL6 | | |
| 7 | 81.8% | DKK3 | M2PK | | IGFB2 | | BDNF | | | TNFa |
| 7 | 81.6% | | M2PK | TGFb | | | BDNF | | IL8 | TNFa |
| 7 | 81.5% | DKK3 | M2PK | | IGFB2 | | BDNF | | | |
| 7 | 81.5% | DKK3 | M2PK | | IGFB2 | | BDNF | IL6 | | TNFa |
| 7 | 81.4% | | M2PK | TGFb | IGFB2 | | BDNF | | IL8 | TNFa |
| 7 | 81.4% | DKK3 | M2PK | TGFb | IGFB2 | | BDNF | | IL8 | TNFa |
| 7 | 81.4% | DKK3 | M2PK | | IGFB2 | | BDNF | | | TNFa |
| 7 | 81.4% | DKK3 | M2PK | | IGFB2 | | BDNF | | IL8 | TNFa |
| 7 | 81.4% | DKK3 | M2PK | TGFb | IGFB2 | | BDNF | IL6 | | |
| 7 | 81.3% | | M2PK | TGFb | | | BDNF | | IL8 | TNFa |
| 7 | 81.3% | DKK3 | M2PK | TGFb | IGFB2 | | BDNF | | | |
| 7 | 81.2% | DKK3 | M2PK | | IGFB2 | | BDNF | | | TNFa |
| 7 | 81.1% | DKK3 | M2PK | TGFb | IGFB2 | | BDNF | | | |
| 7 | 81.0% | DKK3 | M2PK | | IGFB2 | | BDNF | | IL8 | TNFa |
| 7 | 81.0% | DKK3 | M2PK | | IGFB2 | | BDNF | | IL8 | |
| 6 | 80.4% | DKK3 | M2PK | | IGFB2 | | BDNF | | | TNFa |
| 6 | 80.4% | DKK3 | M2PK | | IGFB2 | | BDNF | | | |
| 6 | 80.4% | DKK3 | M2PK | | IGFB2 | | BDNF | | | TNFa |
| 6 | 80.4% | DKK3 | M2PK | | IGFB2 | | BDNF | | | TNFa |
| 6 | 80.4% | DKK3 | M2PK | | IGFB2 | | BDNF | IL6 | | TNFa |
| 6 | 80.4% | DKK3 | M2PK | TGFb | | | BDNF | | IL8 | |
| 6 | 79.9% | | M2PK | TGFb | | | BDNF | | IL8 | TNFa |
| 6 | 79.9% | | M2PK | | | | BDNF | | IL8 | TNFa |
| 6 | 79.9% | | M2PK | TGFb | | | BDNF | | IL8 | TNFa |
| 6 | 79.9% | DKK3 | M2PK | | | | BDNF | | IL8 | TNFa |
| 6 | 79.9% | DKK3 | M2PK | | IGFB2 | | BDNF | | IL8 | TNFa |
| 6 | 79.8% | | M2PK | | | | BDNF | | IL8 | TNFa |
| 6 | 79.6% | | M2PK | TGFb | | | BDNF | | IL8 | TNFa |
| 6 | 79.5% | | M2PK | | | | BDNF | | IL8 | TNFa |
| 6 | 79.5% | | M2PK | | | | BDNF | IL6 | IL8 | TNFa |
| 6 | 79.5% | DKK3 | M2PK | | | | BDNF | | IL8 | TNFa |
| 6 | 79.5% | DKK3 | M2PK | | | | BDNF | | IL8 | TNFa |
| 6 | 79.5% | DKK3 | M2PK | | | | BDNF | | IL8 | TNFa |
| 6 | 79.5% | DKK3 | M2PK | | | | BDNF | IL6 | IL8 | TNFa |
| 6 | 79.3% | | M2PK | | IGFB2 | | BDNF | | IL8 | TNFa |
| 5 | 80.5% | | M2PK | | | | BDNF | | IL8 | TNFa |
| 5 | 80.0% | DKK3 | M2PK | | | | BDNF | | IL8 | |
| 5 | 79.9% | DKK3 | M2PK | TGFb | | | BDNF | | IL8 | |
| 5 | 79.3% | | M2PK | TGFb | | | BDNF | | IL8 | TNFa |
| 5 | 79.3% | DKK3 | M2PK | | IGFB2 | | BDNF | | | TNFa |
| 5 | 79.2% | DKK3 | M2PK | | | | BDNF | | IL8 | |
| 5 | 78.9% | DKK3 | M2PK | | | | BDNF | | IL8 | TNFa |
| 5 | 78.8% | | M2PK | | IGFB2 | | BDNF | | IL8 | |
| 5 | 78.4% | DKK3 | M2PK | | | | BDNF | | IL8 | |
| 5 | 77.8% | | M2PK | | | | BDNF | | IL8 | TNFa |
| 5 | 77.8% | | M2PK | | | | BDNF | | IL8 | TNFa |
| 5 | 77.8% | | M2PK | | | | BDNF | IL6 | IL8 | TNFa |
| 5 | 77.8% | DKK3 | M2PK | | | | BDNF | | | |
| 5 | 77.7% | | M2PK | | | | BDNF | | IL8 | TNFa |
| 5 | 77.7% | DKK3 | M2PK | | | TIMP1 | BDNF | | IL8 | |
| 5 | 77.6% | | M2PK | | | | BDNF | | IL8 | TNFa |
| 5 | 77.5% | DKK3 | M2PK | | | | BDNF | | | |
| 5 | 77.5% | DKK3 | M2PK | | | | BDNF | | IL8 | |
| 5 | 77.3% | | M2PK | | | | BDNF | | IL8 | TNFa |
| 5 | 77.3% | DKK3 | M2PK | | | | BDNF | | | |
| 4 | 77.8% | | M2PK | | | | BDNF | | IL8 | TNFa |
| 4 | 77.3% | | M2PK | | | | BDNF | | IL8 | |
| 4 | 77.3% | DKK3 | M2PK | | | | BDNF | | IL8 | |
| 4 | 76.8% | DKK3 | M2PK | | | | BDNF | | | |
| 4 | 76.1% | | M2PK | | IGFB2 | | BDNF | | IL8 | |
| 4 | 75.5% | | M2PK | | | TIMP1 | BDNF | | IL8 | |
| 4 | 75.4% | | M2PK | | | | BDNF | | | TNFa |
| 4 | 75.1% | | M2PK | | | | BDNF | | IL8 | |
| 4 | 75.0% | | M2PK | | IGFB2 | | BDNF | | | TNFa |
| 4 | 75.0% | | M2PK | | | | BDNF | | IL8 | |
| 4 | 74.6% | DKK3 | M2PK | | | | BDNF | | | TNFa |
| 4 | 74.5% | | M2PK | | | | BDNF | | IL8 | |
| 4 | 74.5% | DKK3 | M2PK | | IGFB2 | | | | | |
| 4 | 74.5% | DKK3 | M2PK | | IGFB2 | | BDNF | | | |
| 4 | 74.3% | | M2PK | | | | BDNF | | IL8 | |
| 4 | 74.2% | | M2PK | | | | BDNF | | IL8 | |
| 4 | 74.2% | DKK3 | M2PK | | | | BDNF | | | |
| 4 | 74.1% | | M2PK | | | | BDNF | | IL8 | |
| 4 | 74.1% | | M2PK | | | | BDNF | | IL8 | |
| 4 | 73.8% | | M2PK | | | | BDNF | | | |
| 3 | 74.6% | | M2PK | | | | BDNF | | IL8 | |
| 3 | 72.4% | | M2PK | | | | BDNF | | | TNFa |
| 3 | 71.4% | DKK3 | M2PK | | | | BDNF | | | |
| 3 | 69.9% | DKK3 | M2PK | | | | | | | |
| 3 | 69.2% | DKK3 | M2PK | | | | | | | |
| 3 | 69.0% | | M2PK | | | | | | | TNFa |
| 3 | 68.5% | | M2PK | | IGFB2 | | BDNF | | | |
| 3 | 68.3% | DKK3 | M2PK | | IGFB2 | | | | | |
| 3 | 68.1% | | M2PK | | | | | | IL8 | TNFa |
| 3 | 68.1% | | M2PK | | | | BDNF | | | |
| 3 | 67.4% | | M2PK | | IGFB2 | | | | IL8 | |
| 3 | 67.2% | | M2PK | | | | | | | TNFa |
| 3 | 67.0% | | M2PK | | | | BDNF | | | |
| 3 | 66.8% | | M2PK | | | | | | IL8 | |
| 3 | 66.5% | | M2PK | | | | BDNF | | | |
| 3 | 66.3% | | M2PK | | | TIMP1 | BDNF | | | |
| 3 | 66.3% | | M2PK | TGFb | | | BDNF | | | |
| 3 | 65.9% | | M2PK | | | | | | | |
| 3 | 65.8% | DKK3 | M2PK | | | | | | | |
| 3 | 65.8% | | M2PK | | | | BDNF | | | |

| # | IGFII | Lipocalin | M30 | M65 | Mac2BP | MMP1 | MMP7 | MIP1B | IL13 |
|---|---|---|---|---|---|---|---|---|---|
| 7 | | | | | Mac2BP | | | MIP1B | |
| 7 | | Lipocalin | | | | | MMP7 | | |
| 7 | | | | M65 | | MMP1 | | | |
| 7 | IGFII | | | | | | | | |
| 7 | | | | | Mac2BP | | MMP7 | | |
| 7 | IGFII | | | M65 | | | | | |
| 7 | | | | M65 | | | MMP7 | | |
| 7 | | | | | Mac2BP | MMP1 | | MIP1B | |
| 7 | | Lipocalin | | | | | | | |
| 7 | | Lipocalin | | | | | | | |
| 7 | | | | | | | | | |
| 7 | IGFII | Lipocalin | | | | | | | |
| 7 | IGFII | | | | | | | | |
| 7 | | Lipocalin | | | | | | | |
| 7 | | | | | | | MMP7 | MIP1B | |
| 7 | IGFII | Lipocalin | | | | | | | |
| 7 | | | | M65 | | | | MIP1B | |
| 7 | IGFII | | | | | | MMP7 | | |
| 7 | | | | | | | | MIP1B | |
| 7 | | | | | Mac2BP | | | MIP1B | |
| 6 | | | | | | | | MIP1B | |
| 6 | | | | | | MMP1 | | MIP1B | |
| 6 | | | | | Mac2BP | | | | |
| 6 | | Lipocalin | | | | | | | |
| 6 | | | | | | | | | |
| 6 | | Lipocalin | | | | | | | |
| 6 | | | | | | | | MIP1B | |
| 6 | IGFII | | | | | | | MIP1B | |
| 6 | | | M30 | | | | | | |
| 6 | IGFII | | | | | | | | |
| 6 | | | | | | | | | |
| 6 | | | | | | | MMP7 | MIP1B | |
| 6 | | | | M65 | | | | | |
| 6 | | | | | Mac2BP | | | MIP1B | |
| 6 | | | | | | | | MIP1B | |
| 6 | | | | | | | | MIP1B | |
| 6 | | | | | | MMP1 | | | |
| 6 | | | M30 | | | | | | |
| 6 | | | | | | | | | |
| 6 | | | | | | | | MIP1B | |
| 5 | | | | | | | | MIP1B | |
| 5 | | Lipocalin | | | | | | | |
| 5 | | | | | | | | | |
| 5 | | | | | | | | | |
| 5 | | | | | | | | | |
| 5 | | | | | | | MMP7 | | |
| 5 | | | | | | | | | |
| 5 | | | | | | | | MIP1B | |
| 5 | | | | | Mac2BP | | | | |
| 5 | | | | | Mac2BP | | | | |
| 5 | | | M30 | | | | | | |
| 5 | | | | | | | | | |
| 5 | | Lipocalin | | | | MMP1 | | | |
| 5 | IGFII | | | | | | | | |
| 5 | | | | | | | | | |
| 5 | | | | | | | MMP7 | | |
| 5 | | | M30 | M65 | | | | | |
| 5 | | | | M65 | | | | | |
| 5 | | Lipocalin | | | | | | | |
| 5 | | | | | Mac2BP | MMP1 | | | |
| 4 | | | | | | | | | |
| 4 | | | | | | | | MIP1B | |
| 4 | | | | | | | | | |
| 4 | | | | | | MMP1 | | | |
| 4 | | | | | | | | | |
| 4 | | | | | | | | | |
| 4 | | | | | | | MMP7 | | |
| 4 | | | | | | MMP1 | | | |
| 4 | | | | | | | | | |
| 4 | IGFII | | | | | | | | |
| 4 | | | | | | | | | |
| 4 | | | | | | | | | IL13 |
| 4 | | | | | | | MMP7 | | |
| 4 | | | | | | | | | |
| 4 | | | | | | | MMP7 | | |
| 4 | | | | M65 | | | | | |
| 4 | | | | M65 | | | | | |
| 4 | | | | | Mac2BP | | | | |
| 4 | | | M30 | | | | | | |
| 4 | | | | | | | MMP7 | MIP1B | |
| 3 | | | | | | | | | |
| 3 | | | | | | | | | |
| 3 | | | | | | | | | |
| 3 | IGFII | | | | | | | | |
| 3 | | | | | | | MMP7 | | |
| 3 | | | | | | | | | IL13 |
| 3 | | | | | | | | | |
| 3 | | | | | | | | | |
| 3 | | | | | | | | | |
| 3 | | | | | Mac2BP | | | | |
| 3 | | | | | | | | | |
| 3 | | | | | | | MMP7 | | |
| 3 | | | | | | | | MIP1B | |
| 3 | | | | | | | | | IL13 |
| 3 | | | | | | MMP1 | | | |
| 3 | | | | | | | | | |
| 3 | | | | | | | | | |
| 3 | IGFII | | | | | | | | IL13 |
| 3 | | | | | | MMP1 | | | |
| 3 | IGFII | | | | | | | | |

TABLE 62
Top 20, 7 to 3 biomarker combinations which may be used to differentiate colorectal cancer
subjects (both genders, age also included in analysis) from controls at 95% specificity.

| # | Sens. | Dkk3 | M2PK | TGFb | IGFBP2 | TIMP1 | BDNF | IL6 | IL8 | TNFa | IGFII |
|---|---|---|---|---|---|---|---|---|---|---|---|
| 7 | 82.1% | DKK3 | M2PK | | IGFBP2 | | BDNF | | | | |
| 7 | 81.9% | DKK3 | M2PK | | IGFBP2 | | BDNF | | IL8 | | |
| 7 | 81.5% | DKK3 | M2PK | | IGFBP2 | | BDNF | IL6 | | | |
| 7 | 81.5% | DKK3 | M2PK | | IGFBP2 | | BDNF | | IL8 | | |
| 7 | 81.4% | | M2PK | TGFb | IGFBP2 | | BDNF | | IL8 | TNFa | |
| 7 | 81.3% | DKK3 | M2PK | TGFb | IGFBP2 | | BDNF | | | | IGFII |
| 7 | 81.3% | DKK3 | M2PK | | IGFBP2 | | BDNF | | | | |
| 7 | 81.3% | DKK3 | M2PK | | IGFBP2 | | BDNF | IL6 | | | |
| 7 | 81.0% | DKK3 | M2PK | | IGFBP2 | | BDNF | IL6 | | | |
| 7 | 81.0% | DKK3 | M2PK | | IGFBP2 | | BDNF | | IL8 | TNFa | |
| 7 | 81.0% | DKK3 | M2PK | | IGFBP2 | | BDNF | | | | |
| 7 | 80.9% | | M2PK | TGFb | IGFBP2 | | BDNF | | IL8 | TNFa | |
| 7 | 80.9% | DKK3 | M2PK | TGFb | IGFBP2 | | BDNF | | IL8 | TNFa | |
| 7 | 80.7% | | M2PK | | | | BDNF | | IL8 | TNFa | |
| 7 | 80.7% | | M2PK | TGFb | IGFBP2 | | BDNF | | IL8 | TNFa | |
| 7 | 80.7% | DKK3 | M2PK | TGFb | | | BDNF | | IL8 | TNFa | |
| 7 | 80.4% | DKK3 | M2PK | | IGFBP2 | | BDNF | IL6 | | | |
| 7 | 80.2% | DKK3 | M2PK | | | | BDNF | | IL8 | TNFa | |
| 7 | 80.2% | DKK3 | M2PK | | IGFBP2 | | BDNF | IL6 | | | |
| 7 | 80.1% | DKK3 | M2PK | | IGFBP2 | | BDNF | | | | |
| 6 | 80.2% | DKK3 | M2PK | TGFb | IGFBP2 | | BDNF | | | | IGFII |
| 6 | 79.9% | | M2PK | TGFb | | | BDNF | | IL8 | TNFa | |
| 6 | 79.7% | DKK3 | M2PK | | | | BDNF | | IL8 | TNFa | |
| 6 | 79.3% | DKK3 | M2PK | | IGFBP2 | | BDNF | | IL8 | TNFa | |
| 6 | 79.3% | DKK3 | M2PK | | IGFBP2 | | BDNF | | | | |
| 6 | 79.2% | | M2PK | | | | BDNF | | IL8 | TNFa | |
| 6 | 79.2% | | M2PK | TGFb | IGFBP2 | | BDNF | | IL8 | TNFa | |
| 6 | 79.2% | DKK3 | M2PK | | IGFBP2 | | BDNF | | IL8 | | IGFII |
| 6 | 79.1% | | M2PK | | IGFBP2 | | BDNF | | IL8 | | |
| 6 | 78.9% | | M2PK | | | | BDNF | | IL8 | TNFa | |
| 6 | 78.9% | | M2PK | | | | BDNF | IL6 | IL8 | TNFa | |
| 6 | 78.8% | | M2PK | | | | BDNF | | IL8 | TNFa | IGFII |
| 6 | 78.8% | | M2PK | | IGFBP2 | | BDNF | | IL8 | TNFa | |
| 6 | 78.8% | DKK3 | M2PK | | IGFBP2 | | BDNF | | | | |
| 6 | 78.8% | | M2PK | TGFb | | | BDNF | | IL8 | TNFa | |
| 6 | 78.8% | DKK3 | M2PK | | IGFBP2 | | BDNF | IL6 | | | |
| 6 | 78.5% | DKK3 | M2PK | | IGFBP2 | | BDNF | | | | |
| 6 | 78.3% | DKK3 | M2PK | TGFb | IGFBP2 | | BDNF | | | | |
| 6 | 78.3% | DKK3 | M2PK | | | TIMP1 | BDNF | | IL8 | TNFa | |
| 6 | 78.3% | | M2PK | TGFb | | | BDNF | | IL8 | TNFa | |
| 5 | 79.5% | | M2PK | | | | BDNF | | IL8 | TNFa | |
| 5 | 78.3% | | M2PK | TGFb | | | BDNF | | IL8 | TNFa | |
| 5 | 77.8% | DKK3 | M2PK | | | | BDNF | | IL8 | | |
| 5 | 77.7% | | M2PK | | IGFBP2 | | BDNF | | IL8 | | |
| 5 | 77.6% | | M2PK | | | | BDNF | | IL8 | TNFa | |
| 5 | 77.6% | DKK3 | M2PK | | | | BDNF | | IL8 | | |
| 5 | 77.3% | | M2PK | | | | BDNF | | IL8 | | |
| 5 | 77.3% | | M2PK | | | | BDNF | | IL8 | | |
| 5 | 77.3% | DKK3 | M2PK | | | | BDNF | | IL8 | TNFa | |
| 5 | 77.2% | | M2PK | | | | BDNF | | IL8 | TNFa | IGFII |
| 5 | 77.2% | DKK3 | M2PK | | | | BDNF | | IL8 | | IGFII |
| 5 | 76.8% | DKK3 | M2PK | | | | BDNF | | IL8 | | |
| 5 | 76.8% | DKK3 | M2PK | | | | BDNF | | | | |
| 5 | 76.8% | DKK3 | M2PK | | | | BDNF | | | | |
| 5 | 76.6% | | M2PK | | IGFBP2 | | BDNF | IL6 | | | |
| 5 | 76.6% | DKK3 | M2PK | | | | BDNF | | | | IGFII |
| 5 | 76.6% | DKK3 | M2PK | | IGFBP2 | | BDNF | | | | |
| 5 | 76.6% | DKK3 | M2PK | | IGFBP2 | | BDNF | IL6 | | | |
| 5 | 76.2% | | M2PK | | | | BDNF | | IL8 | TNFa | |
| 5 | 76.2% | DKK3 | M2PK | | IGFBP2 | | | | | | IGFII |
| 4 | 77.8% | DKK3 | M2PK | | | | BDNF | | IL8 | | |
| 4 | 76.8% | | M2PK | | | | BDNF | | IL8 | | |
| 4 | 76.2% | | M2PK | | | | BDNF | | IL8 | TNFa | |
| 4 | 75.7% | DKK3 | M2PK | | | | BDNF | | | | |
| 4 | 75.7% | DKK3 | M2PK | | IGFBP2 | | | | | | IGFII |
| 4 | 75.5% | | M2PK | | IGFBP2 | | BDNF | | | TNFa | |
| 4 | 75.0% | | M2PK | | IGFBP2 | | BDNF | | IL8 | | |
| 4 | 74.6% | DKK3 | M2PK | | | | BDNF | | | TNFa | |
| 4 | 74.5% | | M2PK | | | | BDNF | | IL8 | | |
| 4 | 74.5% | | M2PK | | | | BDNF | | IL8 | | IGFII |
| 4 | 74.1% | | M2PK | | | | BDNF | | IL8 | | |
| 4 | 73.9% | | M2PK | | | TIMP1 | BDNF | | IL8 | | |
| 4 | 73.9% | DKK3 | M2PK | | IGFBP2 | | | | | | |
| 4 | 73.9% | DKK3 | M2PK | | IGFBP2 | | | | IL8 | | |
| 4 | 73.6% | DKK3 | M2PK | | | | BDNF | | | | |
| 4 | 73.0% | | M2PK | | | | BDNF | | IL8 | | |
| 4 | 72.8% | | M2PK | | IGFBP2 | | BDNF | | | | |
| 4 | 72.8% | | M2PK | TGFb | | | BDNF | | IL8 | | |
| 4 | 72.8% | DKK3 | M2PK | | IGFBP2 | | BDNF | | | | |
| 4 | 72.7% | | M2PK | | | | BDNF | | | TNFa | |
| 3 | 72.4% | | M2PK | | | | BDNF | | IL8 | | |
| 3 | 71.4% | DKK3 | M2PK | | | | BDNF | | | | |
| 3 | 70.4% | DKK3 | M2PK | | | | | | | | IGFII |
| 3 | 69.0% | | M2PK | | | | | | | TNFa | |
| 3 | 67.7% | DKK3 | M2PK | | IGFBP2 | | | | | | |
| 3 | 67.6% | DKK3 | M2PK | | | | | | | | |
| 3 | 67.2% | | M2PK | | | | | | | TNFa | |
| 3 | 66.3% | DKK3 | M2PK | | | | | | | | |
| 3 | 66.3% | | M2PK | | | | | | | | |
| 3 | 65.9% | | M2PK | | | | BDNF | | | TNFa | |
| 3 | 65.8% | | M2PK | | IGFBP2 | | | | | TNFa | |
| 3 | 65.8% | | M2PK | | IGFBP2 | | | | | | |
| 3 | 65.4% | | M2PK | | | | | | | | IGFII |
| 3 | 65.4% | | M2PK | | | | BDNF | | | | |
| 3 | 65.4% | | M2PK | | IGFBP2 | | | | | | IGFII |
| 3 | 65.1% | DKK3 | M2PK | TGFb | | | | | | | |
| 3 | 64.9% | | M2PK | TGFb | IGFBP2 | | | | | | |
| 3 | 64.7% | | M2PK | | IGFBP2 | | BDNF | | | | |
| 3 | 64.3% | | M2PK | | | | BDNF | | | | |
| 3 | 64.1% | | M2PK | | | TIMP1 | BDNF | | | | |

| # | Lipocalin | M30 | M65 | Mac2BP | MMP1 | MMP7 | MIP1B | Age | IL13 |
|---|---|---|---|---|---|---|---|---|---|
| 7 | | | | Mac2BP | MMP1 | | MIP1B | Age | |
| 7 | | | | | | MMP7 | MIP1B | Age | |
| 7 | | | | Mac2BP | | | MIP1B | Age | |
| 7 | Lipocalin | | | | MMP1 | | | Age | |
| 7 | | | | | | | MIP1B | Age | |
| 7 | Lipocalin | | | | | | | Age | |
| 7 | | | | Mac2BP | MMP1 | MMP7 | | Age | |
| 7 | | | | Mac2BP | | MMP7 | | Age | |
| 7 | Lipocalin | | | | | | MIP1B | Age | |
| 7 | | | | | MMP1 | | | Age | |
| 7 | Lipocalin | | | Mac2BP | MMP1 | | | Age | |
| 7 | Lipocalin | | | | | | | Age | |
| 7 | | | | | | | | Age | |
| 7 | | | M65 | | | | MIP1B | Age | IL13 |
| 7 | | | | | | MMP7 | | Age | |
| 7 | | | M65 | | | | | Age | |
| 7 | Lipocalin | | | Mac2BP | | | | Age | |
| 7 | | | M65 | | | | MIP1B | Age | |
| 7 | Lipocalin | | | | | MMP7 | | Age | |
| 7 | | | M65 | Mac2BP | MMP1 | | | Age | |
| 6 | | | | | | | | Age | |
| 6 | | | | | | | MIP1B | Age | |
| 6 | | | M65 | | | | | Age | |
| 6 | | | | | | | | Age | |
| 6 | | | | Mac2BP | MMP1 | | | Age | |
| 6 | | | | | | MMP7 | MIP1B | Age | |
| 6 | | | | | | | | Age | |
| 6 | | | | | | | | Age | |
| 6 | | | | | | MMP7 | MIP1B | Age | |
| 6 | | | | Mac2BP | | | MIP1B | Age | |
| 6 | | | | | | | MIP1B | Age | |
| 6 | | | | | | | MIP1B | Age | |
| 6 | | | | | | | MIP1B | Age | |
| 6 | | | | Mac2BP | | | MIP1B | Age | |
| 6 | | M30 | | | | | | Age | |
| 6 | | | | Mac2BP | | | | Age | |
| 6 | | | M65 | | | | MIP1B | Age | |
| 6 | | | M65 | | | | | Age | |
| 6 | | | | | | | | Age | |
| 6 | | | | Mac2BP | | | | Age | |
| 5 | | | | | | | MIP1B | Age | |
| 5 | | | | | | | | Age | |
| 5 | | | | Mac2BP | | | | Age | |
| 5 | | | | | | | MIP1B | Age | |
| 5 | | | | | | MMP7 | | Age | |
| 5 | | | | | | MMP7 | | Age | |
| 5 | | | | | MMP1 | | MIP1B | Age | |
| 5 | | | | Mac2BP | | | MIP1B | Age | |
| 5 | | | | | | | | Age | |
| 5 | | | | | | | | Age | |
| 5 | | | | | | | | Age | |
| 5 | | | | | | | MIP1B | Age | |
| 5 | | | | Mac2BP | MMP1 | | | Age | |
| 5 | Lipocalin | | | | MMP1 | | | Age | |
| 5 | | | | | | | MIP1B | Age | |
| 5 | | | | | MMP1 | | | Age | |
| 5 | Lipocalin | | | | | | | Age | |
| 5 | | | | | | | | Age | |
| 5 | Lipocalin | | | | | | | Age | |
| 5 | | M30 | | | | | | Age | |
| 4 | | | | | | | | Age | |
| 4 | | | | | | | MIP1B | Age | |
| 4 | | | | | | | | Age | |
| 4 | | | | | MMP1 | | | Age | |
| 4 | | | | | | | | Age | |
| 4 | | | | | | | | Age | |
| 4 | | | | | | | | Age | |
| 4 | | | | | | | | Age | |
| 4 | | | | | | | | Age | IL13 |
| 4 | | | | | | | | Age | |
| 4 | | | | Mac2BP | | | | Age | |
| 4 | | | | | | | | Age | |
| 4 | | | | | | MMP7 | | Age | |
| 4 | | | | | | | | Age | |
| 4 | | | M65 | | | | | Age | |
| 4 | | | | | MMP1 | | | Age | |
| 4 | | | | | | | MIP1B | Age | |
| 4 | | | | | | | | Age | |
| 4 | | | | | | | | Age | |
| 4 | | | | | | MMP7 | | Age | |
| 3 | | | | | | | | Age | |
| 3 | | | | | | | | Age | |
| 3 | | | | | | | | Age | |
| 3 | | | | | | | | Age | IL13 |
| 3 | | | | | | | | Age | |
| 3 | | | | | | MMP7 | | Age | |
| 3 | | | | | | MMP7 | | Age | |
| 3 | | | | | MMP1 | | | Age | |
| 3 | | | | | | MMP7 | | Age | IL13 |
| 3 | | | | | | | | Age | |
| 3 | | | | | | | | Age | |
| 3 | | | | | | MMP7 | | Age | |
| 3 | | | | | | | | Age | IL13 |
| 3 | | | | Mac2BP | | | | Age | |
| 3 | | | | | | | | Age | |
| 3 | | | | | | | | Age | |
| 3 | | | | | | | | Age | |
| 3 | | | | | | | | Age | |
| 3 | | | | | | | MIP1B | Age | |
| 3 | | | | | | | | Age | |

TABLE 63
Top 20, 7 to 3 biomarker combinations which may be used to differentiate colorectal
cancer subjects (female only, age not considered) from controls at 95% specificity.

| # | Sens. | Dkk3 | M2PK | TGFb | IGFBP2 | TIMP1 | BDNF | IL6 | IL8 | TNFa | IGFII |
|---|---|---|---|---|---|---|---|---|---|---|---|
| 7 | 90.8% | | M2PK | | IGFBP2 | | BDNF | | IL8 | | IGFII |
| 7 | 90.8% | | M2PK | | | | BDNF | | IL8 | TNFa | |
| 7 | 90.8% | | M2PK | | | | BDNF | | IL8 | TNFa | |
| 7 | 90.8% | | M2PK | TGFb | | | BDNF | | IL8 | | |
| 7 | 90.8% | | M2PK | | | | BDNF | | IL8 | | IGFII |
| 7 | 90.7% | | M2PK | | | | BDNF | | IL8 | | |
| 7 | 90.7% | | M2PK | | | | BDNF | | IL8 | TNFa | |
| 7 | 90.7% | | M2PK | | | | BDNF | IL6 | IL8 | | |
| 7 | 90.7% | | M2PK | | | | BDNF | | IL8 | | |
| 7 | 90.7% | | M2PK | | | | BDNF | | IL8 | | IGFII |
| 7 | 89.7% | DKK3 | M2PK | | | | BDNF | | IL8 | TNFa | |
| 7 | 89.7% | DKK3 | M2PK | TGFb | | | BDNF | | IL8 | | |
| 7 | 89.7% | | M2PK | TGFb | | | BDNF | | IL8 | | |
| 7 | 89.7% | | M2PK | TGFb | | | BDNF | | IL8 | TNFa | |
| 7 | 89.6% | DKK3 | M2PK | | IGFBP2 | | BDNF | | IL8 | | |
| 7 | 89.6% | | M2PK | | | | BDNF | | IL8 | | IGFII |
| 7 | 89.6% | | M2PK | TGFb | | | BDNF | | IL8 | | IGFII |
| 7 | 89.6% | | M2PK | TGFb | | | BDNF | | IL8 | | |
| 7 | 89.6% | | M2PK | | | | BDNF | | IL8 | | |
| 7 | 89.6% | | M2PK | TGFb | | | BDNF | | IL8 | | IGFII |
| 6 | 90.7% | | M2PK | | | | BDNF | | IL8 | | |
| 6 | 89.7% | DKK3 | M2PK | | | | BDNF | | IL8 | | |
| 6 | 89.7% | | M2PK | TGFb | | | BDNF | | IL8 | TNFa | |
| 6 | 89.7% | | M2PK | | | | BDNF | | IL8 | TNFa | |
| 6 | 89.7% | | M2PK | TGFb | | | BDNF | | IL8 | | |
| 6 | 89.7% | | M2PK | TGFb | | | BDNF | | IL8 | TNFa | |
| 6 | 89.7% | | M2PK | TGFb | | | BDNF | IL6 | IL8 | TNFa | |
| 6 | 89.7% | DKK3 | M2PK | TGFb | | | BDNF | | IL8 | | |
| 6 | 89.7% | | M2PK | TGFb | | | BDNF | IL6 | IL8 | | |
| 6 | 89.6% | | M2PK | | | | BDNF | | IL8 | | |
| 6 | 89.6% | | M2PK | | | | BDNF | | IL8 | | IGFII |
| 6 | 89.6% | | M2PK | TGFb | | | BDNF | IL6 | IL8 | | |
| 6 | 89.6% | | M2PK | TGFb | | | BDNF | | IL8 | | |
| 6 | 89.6% | | M2PK | TGFb | | | BDNF | | IL8 | | |
| 6 | 89.6% | | M2PK | TGFb | | | BDNF | | IL8 | | IGFII |
| 6 | 89.6% | | M2PK | | | TIMP1 | BDNF | | IL8 | | |
| 6 | 89.5% | | M2PK | | | | BDNF | | IL8 | | |
| 6 | 89.5% | | M2PK | | | | BDNF | | IL8 | | |
| 6 | 89.5% | | M2PK | | | | BDNF | | IL8 | | IGFII |
| 6 | 89.5% | | M2PK | TGFb | | TIMP1 | BDNF | | IL8 | | |
| 5 | 89.7% | | M2PK | TGFb | | | BDNF | | IL8 | TNFa | |
| 5 | 89.7% | | M2PK | | | | BDNF | | IL8 | TNFa | |
| 5 | 89.7% | | M2PK | TGFb | | | BDNF | | IL8 | | |
| 5 | 89.7% | | M2PK | TGFb | | | BDNF | | IL8 | | |
| 5 | 89.7% | | M2PK | TGFb | | | BDNF | IL6 | IL8 | | |
| 5 | 89.6% | | M2PK | TGFb | | | BDNF | | IL8 | | IGFII |
| 5 | 89.6% | | M2PK | TGFb | | | BDNF | | IL8 | | |
| 5 | 88.5% | | M2PK | | | | BDNF | | IL8 | | |
| 5 | 88.5% | | M2PK | | | | BDNF | | IL8 | | |
| 5 | 88.5% | | M2PK | TGFb | | | BDNF | | IL8 | | |
| 5 | 88.5% | | M2PK | | | | BDNF | | IL8 | | |
| 5 | 88.5% | | M2PK | | | | BDNF | | IL8 | TNFa | |
| 5 | 88.5% | | M2PK | | | | BDNF | IL6 | IL8 | | |
| 5 | 88.5% | | M2PK | | | | BDNF | | IL8 | | |
| 5 | 88.5% | | M2PK | | | | BDNF | IL6 | IL8 | | |
| 5 | 88.3% | | M2PK | | | | BDNF | | IL8 | | IGFII |
| 5 | 87.2% | DKK3 | M2PK | | | | BDNF | | IL8 | | |
| 5 | 87.2% | | M2PK | | | | BDNF | | IL8 | TNFa | |
| 5 | 87.2% | | M2PK | | | | BDNF | | | | |
| 5 | 87.0% | | M2PK | | | | BDNF | | IL8 | | |
| 4 | 89.7% | | M2PK | | | | BDNF | | IL8 | | |
| 4 | 88.5% | | M2PK | | | | BDNF | | IL8 | | |
| 4 | 88.5% | | M2PK | | | | BDNF | IL6 | IL8 | | |
| 4 | 87.2% | | M2PK | | | | BDNF | | IL8 | | |
| 4 | 87.2% | | M2PK | | | | BDNF | | IL8 | | |
| 4 | 85.9% | | M2PK | TGFb | | | BDNF | | IL8 | | |
| 4 | 85.7% | | M2PK | | | | BDNF | | IL8 | | |
| 4 | 85.7% | | M2PK | | | | BDNF | | IL8 | | IGFII |
| 4 | 84.6% | | M2PK | | | | BDNF | | IL8 | TNFa | |
| 4 | 84.6% | | M2PK | TGFb | | | BDNF | | | | |
| 4 | 84.6% | | M2PK | | | | BDNF | | IL8 | | |
| 4 | 83.3% | | M2PK | | | | BDNF | | | | |
| 4 | 83.3% | | M2PK | | | | BDNF | | | | |
| 4 | 83.3% | | M2PK | | | | | IL6 | IL8 | | |
| 4 | 83.1% | | M2PK | | | | | | IL8 | | |
| 4 | 83.1% | | M2PK | | | TIMP1 | BDNF | | IL8 | | |
| 4 | 81.8% | | M2PK | | | | | | IL8 | | |
| 4 | 80.8% | | M2PK | | | | BDNF | IL6 | | | |
| 4 | 80.5% | | M2PK | | IGFBP2 | | BDNF | | IL8 | | |
| 4 | 80.0% | | M2PK | | | | | | IL8 | | |
| m | 85.9% | | M2PK | | | | BDNF | | IL8 | | |
| 3 | 80.8% | | M2PK | | | | | IL6 | IL8 | | |
| 3 | 80.5% | | M2PK | | | | | | IL8 | | |
| 3 | 79.5% | | M2PK | | | | BDNF | | | | |
| 3 | 78.2% | | M2PK | | | | BDNF | | | | |
| 3 | 78.2% | | M2PK | | | | BDNF | | | | |
| 3 | 77.9% | | M2PK | | | | BDNF | | | | |
| 3 | 76.9% | | M2PK | | | | BDNF | | | | |
| 3 | 76.9% | | M2PK | | | | | | IL8 | | |
| 3 | 76.9% | | M2PK | | | | BDNF | IL6 | | | |
| 3 | 76.6% | | M2PK | | | TIMP1 | BDNF | | | | |
| 3 | 75.6% | DKK3 | M2PK | | | | BDNF | | | | |
| 3 | 74.4% | | M2PK | | | | BDNF | | | TNFa | |
| 3 | 74.4% | | M2PK | | | | | IL6 | | | |
| 3 | 74.4% | | M2PK | | | | | | | TNFa | |
| 3 | 74.4% | | M2PK | | | | | | IL8 | | |
| 3 | 74.0% | | M2PK | | | | BDNF | | | | IGFII |
| 3 | 73.4% | | M2PK | | | | | | | | |
| 3 | 73.1% | | M2PK | | | | BDNF | | | | |
| 3 | 73.1% | | M2PK | | | | | IL6 | | | |

| # | Lipocalin | M30 | M65 | Mac2BP | MMP1 | MMP7 | MIP1B | Age | IL13 |
|---|---|---|---|---|---|---|---|---|---|
| 7 | Lipocalin | | | | MMP1 | | | | |
| 7 | | | M65 | Mac2BP | MMP1 | | | | |
| 7 | Lipocalin | | M65 | | MMP1 | | | | |
| 7 | | M30 | M65 | | MMP1 | | | | |
| 7 | | | M65 | Mac2BP | MMP1 | | | | |
| 7 | | M30 | M65 | | | | MIP1B | | IL13 |
| 7 | | M30 | M65 | | | | | | IL13 |
| 7 | | M30 | M65 | | | | | | IL13 |
| 7 | | M30 | M65 | Mac2BP | | | | | IL13 |
| 7 | | M30 | M65 | | | | | | IL13 |
| 7 | | | | Mac2BP | MMP1 | | | | |
| 7 | | | | Mac2BP | MMP1 | | | | |
| 7 | Lipocalin | | | | MMP1 | | MIP1B | | |
| 7 | | | | Mac2BP | MMP1 | | | | |
| 7 | | | | Mac2BP | | | MIP1B | | |
| 7 | | | | Mac2BP | MMP1 | | MIP1B | | |
| 7 | | | | Mac2BP | MMP1 | | | | |
| 7 | | M30 | | | | | MIP1B | | IL13 |
| 7 | | M30 | | Mac2BP | | | MIP1B | | IL13 |
| 7 | | | | | MMP1 | | MIP1B | | |
| 6 | | M30 | M65 | | | | | | IL13 |
| 6 | | | | Mac2BP | MMP1 | | | | |
| 6 | | | | Mac2BP | | | | | |
| 6 | | | | Mac2BP | MMP1 | | | | |
| 6 | | | | Mac2BP | MMP1 | | | | |
| 6 | | | | | MMP1 | | | | |
| 6 | | | | | | | | | |
| 6 | | | | | MMP1 | | | | |
| 6 | | | | Mac2BP | | | | | |
| 6 | | M30 | | | | | MIP1B | | IL13 |
| 6 | | | | | MMP1 | | MIP1B | | |
| 6 | | | | | | | | | IL13 |
| 6 | | | | | | | MIP1B | | IL13 |
| 6 | | M30 | | | | | | | IL13 |
| 6 | | | | | MMP1 | | | | |
| 6 | Lipocalin | | | | MMP1 | | | | |
| 6 | | | M65 | Mac2BP | MMP1 | | | | |
| 6 | | M30 | M65 | | MMP1 | | | | |
| 6 | | M30 | | | | | | | IL13 |
| 6 | | | | | | | | | IL13 |
| 5 | | | | | | | | | |
| 5 | | | | | MMP1 | | | | |
| 5 | | | | | MMP1 | | | | |
| 5 | | | | Mac2BP | | | | | |
| 5 | | | | | | | | | |
| 5 | | | | | | | | | |
| 5 | | | | | | | | | IL13 |
| 5 | | | | | MMP1 | | MIP1B | | |
| 5 | | | | Mac2BP | MMP1 | | | | |
| 5 | | | | | | | MIP1B | | |
| 5 | | | | Mac2BP | | | MIP1B | | |
| 5 | | | | Mac2BP | | | | | |
| 5 | | | | | MMP1 | | | | |
| 5 | Lipocalin | | | | MMP1 | | | | |
| 5 | | | | Mac2BP | | | | | |
| 5 | | | | | MMP1 | | | | |
| 5 | | | | | MMP1 | | | | |
| 5 | | | | | | | MIP1B | | |
| 5 | | | | Mac2BP | MMP1 | | MIP1B | | |
| 5 | | | | | | | MIP1B | | IL13 |
| 4 | | | | | MMP1 | | | | |
| 4 | Lipocalin | | | | | | | | |
| 4 | | | | | | | | | |
| 4 | | | | | | | MIP1B | | |
| 4 | | | | Mac2BP | | | | | |
| 4 | | | | | | | | | |
| 4 | | | | | | | | | IL13 |
| 4 | | | | | | | | | |
| 4 | | | | | | | | | |
| 4 | | | | | MMP1 | | | | |
| 4 | | M30 | | | | | | | |
| 4 | | | | | MMP1 | | MIP1B | | |
| 4 | | | | Mac2BP | MMP1 | | | | |
| 4 | | | | | MMP1 | | | | |
| 4 | | M30 | | | | | | | IL13 |
| 4 | | | | | | | | | |
| 4 | | | | | MMP1 | | | | IL13 |
| 4 | Lipocalin | | | | | | | | |
| 4 | | | | | | | | | |
| 4 | | | M65 | | | | | | IL13 |
| m | | | | | | | | | |
| 3 | | | | | | | | | |
| 3 | | | | | | | | | IL13 |
| 3 | | | | Mac2BP | | | | | |
| 3 | | | | | MMP1 | | | | |
| 3 | Lipocalin | | | | | | | | |
| 3 | | | | | | | | | IL13 |
| 3 | | | | | | | MIP1B | | |
| 3 | | M30 | | | | | | | |
| 3 | | | | | | | | | |
| 3 | | | | | | | | | |
| 3 | | | | | | | | | |
| 3 | | | | | | | | | |
| 3 | | | | | MMP1 | | | | |
| 3 | | | | | MMP1 | | | | |
| 3 | | | | | MMP1 | | | | |
| 3 | | | | | | | | | |
| 3 | | | | | MMP1 | | MIP1B | | |
| 3 | | M30 | | | | | | | |
| 3 | | | | Mac2BP | | | | | |

TABLE 64
Top 20, 7 to 3 biomarker combinations which may be used to differentiate colorectal cancer
subjects (female only, age also included in analysis) from controls at 95% specificity.

| # | Sens. | Dkk3 | M2PK | TGFb | IGFBP2 | TIMP1 | BDNF | IL6 | IL8 | TNFa | IGFII |
|---|---|---|---|---|---|---|---|---|---|---|---|
| 7 | 92.2% | | M2PK | TGFb | IGFBP2 | | BDNF | | IL8 | | |
| 7 | 92.1% | | M2PK | | IGFBP2 | | BDNF | | IL8 | | IGFII |
| 7 | 92.0% | | M2PK | | | | BDNF | IL6 | IL8 | | |
| 7 | 90.9% | | M2PK | | IGFBP2 | | BDNF | | IL8 | | |
| 7 | 90.7% | DKK3 | M2PK | | IGFBP2 | | BDNF | | | | |
| 7 | 90.7% | | M2PK | | | | BDNF | | IL8 | TNFa | |
| 7 | 90.7% | | M2PK | | | | BDNF | | IL8 | | IGFII |
| 7 | 89.6% | | M2PK | | IGFBP2 | | BDNF | IL6 | IL8 | | |
| 7 | 89.6% | | M2PK | TGFb | IGFBP2 | | BDNF | IL6 | IL8 | | |
| 7 | 89.6% | | M2PK | | | | BDNF | | IL8 | TNFa | |
| 7 | 89.6% | | M2PK | TGFb | | | BDNF | | IL8 | | |
| 7 | 89.6% | | M2PK | TGFb | IGFBP2 | | BDNF | IL6 | IL8 | | |
| 7 | 89.5% | | M2PK | | | | BDNF | | | TNFa | |
| 7 | 89.5% | | M2PK | | | | BDNF | | | | |
| 7 | 89.5% | | M2PK | | IGFBP2 | TIMP1 | BDNF | | IL8 | | |
| 7 | 89.5% | | M2PK | | | | BDNF | | IL8 | | |
| 7 | 89.5% | | M2PK | | | TIMP1 | BDNF | | IL8 | | |
| 7 | 89.5% | | M2PK | | | | BDNF | | IL8 | | IGFII |
| 7 | 89.5% | | M2PK | | | | BDNF | | IL8 | | |
| 7 | 89.5% | DKK3 | M2PK | | | | BDNF | | IL8 | | |
| 6 | 92.2% | | M2PK | | IGFBP2 | | BDNF | | IL8 | | |
| 6 | 89.6% | | M2PK | | | | BDNF | | IL8 | | |
| 6 | 89.6% | | M2PK | TGFb | | | BDNF | | IL8 | | |
| 6 | 89.6% | | M2PK | | | TIMP1 | BDNF | | IL8 | | |
| 6 | 89.5% | | M2PK | | | | BDNF | | IL8 | | IGFII |
| 6 | 89.3% | | M2PK | | | | BDNF | | IL8 | | |
| 6 | 88.5% | | M2PK | TGFb | | | BDNF | | IL8 | TNFa | |
| 6 | 88.5% | | M2PK | | | | BDNF | | IL8 | TNFa | |
| 6 | 88.5% | | M2PK | TGFb | | | BDNF | | IL8 | TNFa | |
| 6 | 88.5% | | M2PK | TGFb | | | BDNF | | IL8 | | |
| 6 | 88.5% | | M2PK | | | | BDNF | | IL8 | | |
| 6 | 88.5% | | M2PK | TGFb | | | BDNF | | | | |
| 6 | 88.3% | | M2PK | | IGFBP2 | | | IL6 | IL8 | | |
| 6 | 88.0% | | M2PK | | IGFBP2 | | | IL6 | IL8 | | |
| 6 | 88.0% | | M2PK | TGFb | | | BDNF | | IL8 | | |
| 6 | 87.2% | | M2PK | | | | BDNF | IL6 | IL8 | | |
| 6 | 87.2% | | M2PK | | | | BDNF | | IL8 | TNFa | |
| 6 | 87.2% | | M2PK | TGFb | | | BDNF | | IL8 | TNFa | |
| 6 | 87.2% | | M2PK | TGFb | | | BDNF | | IL8 | | |
| 6 | 87.2% | | M2PK | TGFb | | | BDNF | | IL8 | | |
| 5 | 88.5% | | M2PK | | | | BDNF | IL6 | IL8 | | |
| 5 | 88.5% | | M2PK | | | | BDNF | IL6 | IL8 | | |
| 5 | 88.5% | | M2PK | | | | BDNF | | IL8 | | |
| 5 | 88.3% | | M2PK | | IGFBP2 | | | IL6 | IL8 | | |
| 5 | 87.2% | | M2PK | | | | BDNF | | IL8 | | |
| 5 | 87.2% | | M2PK | TGFb | | | BDNF | | IL8 | | |
| 5 | 87.2% | | M2PK | | | | BDNF | | IL8 | TNFa | |
| 5 | 87.2% | | M2PK | TGFb | | | BDNF | | IL8 | | |
| 5 | 87.2% | | M2PK | TGFb | | | BDNF | | IL8 | | |
| 5 | 87.2% | | M2PK | TGFb | | | BDNF | | | | |
| 5 | 87.0% | | M2PK | | | | BDNF | | IL8 | | |
| 5 | 87.0% | | M2PK | | | | BDNF | | IL8 | TNFa | |
| 5 | 87.0% | | M2PK | | | | BDNF | | IL8 | | |
| 5 | 87.0% | | M2PK | TGFb | | | BDNF | | IL8 | | |
| 5 | 86.8% | | M2PK | | | | BDNF | | | | |
| 5 | 86.8% | | M2PK | | | | BDNF | | IL8 | | |
| 5 | 85.9% | | M2PK | | | | BDNF | | IL8 | | |
| 5 | 85.9% | | M2PK | | | | BDNF | | IL8 | | |
| 5 | 85.9% | | M2PK | | | | BDNF | | IL8 | TNFa | |
| 5 | 85.9% | | M2PK | | | | BDNF | | | | |
| 4 | 88.5% | | M2PK | | | | BDNF | | IL8 | | |
| 4 | 85.9% | | M2PK | | | | BDNF | | IL8 | | |
| 4 | 85.9% | | M2PK | TGFb | | | BDNF | | IL8 | | |
| 4 | 84.6% | | M2PK | | | | BDNF | | IL8 | | |
| 4 | 84.6% | | M2PK | | | | BDNF | | IL8 | | |
| 4 | 84.4% | | M2PK | | | | BDNF | | IL8 | | |
| 4 | 83.3% | | M2PK | | | | BDNF | | IL8 | | |
| 4 | 83.3% | | M2PK | TGFb | | | BDNF | | | | |
| 4 | 83.1% | DKK3 | M2PK | | IGFBP2 | | | | IL8 | | |
| 4 | 82.1% | | M2PK | | | | BDNF | | | | |
| 4 | 82.1% | | M2PK | | | | | IL6 | IL8 | | |
| 4 | 82.1% | | M2PK | | | | BDNF | | | | |
| 4 | 81.8% | | M2PK | | | TIMP1 | BDNF | | IL8 | | |
| 4 | 81.8% | | M2PK | | IGFBP2 | | | IL6 | IL8 | | |
| 4 | 81.8% | | M2PK | | | | BDNF | | IL8 | | IGFII |
| 4 | 81.8% | | M2PK | | | | | | IL8 | | |
| 4 | 81.8% | | M2PK | | | | | | IL8 | | |
| 4 | 80.8% | | M2PK | | | | BDNF | | IL8 | TNFa | |
| 4 | 80.8% | | M2PK | | | | | IL6 | IL8 | | |
| 4 | 80.8% | | M2PK | | | | BDNF | | | TNFa | |
| 3 | 83.3% | | M2PK | | | | BDNF | | IL8 | | |
| 3 | 79.5% | | M2PK | | | | BDNF | | | | |
| 3 | 79.5% | | M2PK | | | | BDNF | | | | |
| 3 | 79.2% | | M2PK | | | | | | IL8 | | |
| 3 | 77.9% | | M2PK | | IGFBP2 | | | | IL8 | | |
| 3 | 76.9% | | M2PK | | | | BDNF | | | | |
| 3 | 76.9% | | M2PK | | | | BDNF | IL6 | | | |
| 3 | 75.6% | | M2PK | | | | BDNF | | | | |
| 3 | 75.3% | | M2PK | | | | BDNF | | | | |
| 3 | 75.3% | | M2PK | | | TIMP1 | BDNF | | | | |
| 3 | 74.4% | | M2PK | | | | BDNF | | | | |
| 3 | 74.4% | DKK3 | M2PK | | | | BDNF | | | | |
| 3 | 74.4% | | M2PK | | | | | | IL8 | | |
| 3 | 74.4% | | M2PK | | | | | IL6 | | | |
| 3 | 74.0% | | M2PK | | IGFBP2 | | | IL6 | | | |
| 3 | 73.4% | | M2PK | | | | | | | | |
| 3 | 73.1% | | M2PK | | | | BDNF | | | TNFa | |
| 3 | 73.1% | | M2PK | | | | | | | | IGFII |
| 3 | 72.7% | | M2PK | | | | BDNF | | | | IGFII |
| 3 | 71.8% | | M2PK | | | | | | IL8 | | |

| # | Lipocalin | M30 | M65 | Mac2BP | MMP1 | MMP7 | MIP1B | Age | IL13 |
|---|---|---|---|---|---|---|---|---|---|
| 7 | Lipocalin | | | | MMP1 | | | Age | |
| 7 | Lipocalin | | | | MMP1 | | | Age | |
| 7 | | M30 | M65 | | | | | Age | IL13 |
| 7 | Lipocalin | | | Mac2BP | MMP1 | | | Age | |
| 7 | | | M65 | Mac2BP | MMP1 | | | Age | |
| 7 | | M30 | M65 | | | | | Age | IL13 |
| 7 | | M30 | M65 | | | | | Age | IL13 |
| 7 | Lipocalin | | | | MMP1 | | | Age | |
| 7 | Lipocalin | | | | | | | Age | |
| 7 | | M30 | | | | | MIP1B | Age | IL13 |
| 7 | | M30 | | | | | MIP1B | Age | IL13 |
| 7 | | | | Mac2BP | | | | Age | |
| 7 | Lipocalin | | M65 | | MMP1 | | MIP1B | Age | |
| 7 | Lipocalin | | M65 | Mac2BP | MMP1 | | MIP1B | Age | |
| 7 | Lipocalin | | | | MMP1 | | | Age | |
| 7 | | M30 | M65 | Mac2BP | MMP1 | | | Age | |
| 7 | | M30 | | | | | MIP1B | Age | IL13 |
| 7 | | M30 | | | | | MIP1B | Age | IL13 |
| 7 | | M30 | M65 | | MMP1 | | MIP1B | Age | |
| 7 | | M30 | M65 | | MMP1 | | | Age | |
| 6 | Lipocalin | | | | MMP1 | | | Age | |
| 6 | | M30 | | | | | MIP1B | Age | IL13 |
| 6 | | M30 | | | | | | Age | IL13 |
| 6 | Lipocalin | | | | MMP1 | | | Age | |
| 6 | | M30 | | | | | | Age | IL13 |
| 6 | | M30 | M65 | | | | | Age | IL13 |
| 6 | | | | Mac2BP | | | | Age | |
| 6 | Lipocalin | | | | MMP1 | | | Age | |
| 6 | | | | | MMP1 | | | Age | |
| 6 | | M30 | | | MMP1 | | | Age | |
| 6 | Lipocalin | M30 | | | MMP1 | | | Age | |
| 6 | | | | Mac2BP | MMP1 | | MIP1B | Age | |
| 6 | Lipocalin | M30 | | | | | | Age | |
| 6 | Lipocalin | | M65 | | | | | Age | |
| 6 | | | M65 | | | | | Age | IL13 |
| 6 | | | | Mac2BP | | | MIP1B | Age | |
| 6 | | | | Mac2BP | MMP1 | | | Age | |
| 6 | | | | | | | MIP1B | Age | |
| 6 | | | | Mac2BP | | | MIP1B | Age | |
| 6 | | | | Mac2BP | MMP1 | | | Age | |
| 5 | | | | | | | MIP1B | Age | |
| 5 | | | | | MMP1 | | | Age | |
| 5 | Lipocalin | | | | MMP1 | | | Age | |
| 5 | Lipocalin | | | | | | | Age | |
| 5 | | | | Mac2BP | MMP1 | | | Age | |
| 5 | | | | | | | MIP1B | Age | |
| 5 | | | | | MMP1 | | | Age | |
| 5 | | | | | MMP1 | | | Age | |
| 5 | | | | Mac2BP | | | | Age | |
| 5 | | | | | MMP1 | | MIP1B | Age | |
| 5 | | | | | | | MIP1B | Age | IL13 |
| 5 | | | | | | | | Age | IL13 |
| 5 | | M30 | | | | | | Age | IL13 |
| 5 | | | | | | | | Age | IL13 |
| 5 | | | M65 | | MMP1 | | MIP1B | Age | |
| 5 | | | M65 | | MMP1 | | | Age | |
| 5 | | | | | MMP1 | | MIP1B | Age | |
| 5 | | | | Mac2BP | | | MIP1B | Age | |
| 5 | | | | Mac2BP | | | | Age | |
| 5 | | | | Mac2BP | MMP1 | | MIP1B | Age | |
| 4 | | | | | MMP1 | | | Age | |
| 4 | | | | | | | MIP1B | Age | |
| 4 | | | | | | | | Age | |
| 4 | | | | Mac2BP | | | | Age | |
| 4 | | M30 | | | | | | Age | |
| 4 | | | | | | | | Age | IL13 |
| 4 | Lipocalin | | | | | | | Age | |
| 4 | | | | | MMP1 | | | Age | |
| 4 | | | | | | | | Age | |
| 4 | | | | | MMP1 | | MIP1B | Age | |
| 4 | | M30 | | | | | | Age | |
| 4 | | | | Mac2BP | MMP1 | | | Age | |
| 4 | | | | | | | | Age | |
| 4 | | | | | | | | Age | |
| 4 | | | | | | | | Age | |
| 4 | | M30 | | | | | | Age | IL13 |
| 4 | | | | | MMP1 | | | Age | IL13 |
| 4 | | | | | | | | Age | |
| 4 | | | | | MMP1 | | | Age | |
| 4 | | | | | MMP1 | | | Age | |
| 3 | | | | | | | | Age | |
| 3 | | | | Mac2BP | | | | Age | |
| 3 | | | | | MMP1 | | | Age | |
| 3 | | | | | | | | Age | IL13 |
| 3 | | | | | | | | Age | |
| 3 | | | | | | | MIP1B | Age | |
| 3 | | | | | | | | Age | |
| 3 | | M30 | | | | | | Age | |
| 3 | | | | | | | | Age | IL13 |
| 3 | | | | | | | | Age | |
| 3 | Lipocalin | | | | | | | Age | |
| 3 | | | | | | | | Age | |
| 3 | | | | | MMP1 | | | Age | |
| 3 | | | | | MMP1 | | | Age | |
| 3 | | | | | | | | Age | |
| 3 | | | | | MMP1 | | MIP1B | Age | |
| 3 | | | | | | | | Age | |
| 3 | | | | | MMP1 | | | Age | |
| 3 | | | | | | | | Age | |
| 3 | | M30 | | | | | | Age | |

TABLE 65
Top 20, 7 to 3 biomarker combinations which may be used to differentiate colorectal
cancer subjects (male only, age not considered) from controls at 95% specificity.

| # | Sens. | Dkk3 | M2PK | TGFb | IGFBP2 | TIMP1 | BDNF | IL6 | IL8 | TNFa | IGFII |
|---|---|---|---|---|---|---|---|---|---|---|---|
| 7 | 82.2% | DKK3 | M2PK | | | TIMP1 | BDNF | | IL8 | TNFa | |
| 7 | 82.2% | DKK3 | M2PK | | IGFBP2 | TIMP1 | BDNF | | | | |
| 7 | 82.2% | DKK3 | M2PK | | | TIMP1 | BDNF | | IL8 | TNFa | |
| 7 | 82.1% | DKK3 | M2PK | | IGFBP2 | | BDNF | | | TNFa | |
| 7 | 82.1% | DKK3 | M2PK | | IGFBP2 | | BDNF | | | TNFa | IGFII |
| 7 | 82.1% | DKK3 | M2PK | | IGFBP2 | | BDNF | | | TNFa | |
| 7 | 82.1% | DKK3 | M2PK | | | TIMP1 | BDNF | | IL8 | TNFa | |
| 7 | 82.1% | DKK3 | M2PK | | IGFBP2 | | BDNF | | | TNFa | |
| 7 | 81.3% | DKK3 | M2PK | | IGFBP2 | | BDNF | | | TNFa | IGFII |
| 7 | 81.1% | DKK3 | M2PK | | IGFBP2 | | BDNF | | | TNFa | |
| 7 | 81.1% | DKK3 | M2PK | | IGFBP2 | | BDNF | | | TNFa | |
| 7 | 81.1% | DKK3 | M2PK | | | | BDNF | | | TNFa | |
| 7 | 81.1% | DKK3 | M2PK | | IGFBP2 | | BDNF | | | TNFa | |
| 7 | 81.1% | DKK3 | M2PK | | IGFBP2 | | BDNF | | | TNFa | |
| 7 | 81.1% | DKK3 | M2PK | TGFb | | TIMP1 | BDNF | | IL8 | TNFa | |
| 7 | 81.1% | DKK3 | M2PK | | | | BDNF | | | TNFa | |
| 7 | 81.0% | DKK3 | M2PK | | IGFBP2 | | BDNF | | | TNFa | |
| 7 | 80.4% | DKK3 | M2PK | | | | BDNF | | IL8 | TNFa | IGFII |
| 7 | 80.4% | DKK3 | M2PK | | IGFBP2 | | BDNF | | | TNFa | IGFII |
| 7 | 80.4% | DKK3 | M2PK | | | TIMP1 | BDNF | | IL8 | TNFa | |
| 6 | 82.2% | DKK3 | M2PK | | | TIMP1 | BDNF | | IL8 | TNFa | |
| 6 | 81.3% | DKK3 | M2PK | | IGFBP2 | | BDNF | | | TNFa | |
| 6 | 81.3% | DKK3 | M2PK | | IGFBP2 | | BDNF | | | TNFa | |
| 6 | 81.1% | DKK3 | M2PK | | IGFBP2 | | BDNF | | | TNFa | |
| 6 | 81.1% | DKK3 | M2PK | | | | BDNF | | | TNFa | |
| 6 | 81.1% | DKK3 | M2PK | | IGFBP2 | | BDNF | | | TNFa | |
| 6 | 80.4% | DKK3 | M2PK | | | | BDNF | | IL8 | TNFa | IGFII |
| 6 | 80.4% | DKK3 | M2PK | | IGFBP2 | | BDNF | | | TNFa | |
| 6 | 80.2% | DKK3 | M2PK | | | | BDNF | | | TNFa | |
| 6 | 79.4% | DKK3 | M2PK | | IGFBP2 | | BDNF | | | TNFa | |
| 6 | 79.4% | DKK3 | M2PK | | IGFBP2 | TIMP1 | BDNF | | | TNFa | |
| 6 | 79.2% | DKK3 | M2PK | | | | BDNF | | | TNFa | IGFII |
| 6 | 79.2% | DKK3 | M2PK | TGFb | | | | | | TNFa | |
| 6 | 79.2% | DKK3 | M2PK | TGFb | | | | | | TNFa | |
| 6 | 79.2% | DKK3 | M2PK | TGFb | | | | | | TNFa | |
| 6 | 79.0% | DKK3 | M2PK | TGFb | IGFBP2 | | | | | TNFa | |
| 6 | 78.5% | DKK3 | M2PK | | IGFBP2 | | BDNF | | | TNFa | |
| 6 | 78.5% | DKK3 | M2PK | | | TIMP1 | BDNF | | IL8 | | |
| 6 | 78.5% | DKK3 | M2PK | | | TIMP1 | BDNF | | IL8 | | |
| 6 | 78.5% | DKK3 | M2PK | | IGFBP2 | TIMP1 | BDNF | | | | |
| 5 | 80.4% | DKK3 | M2PK | | IGFBP2 | | BDNF | | | TNFa | |
| 5 | 79.2% | DKK3 | M2PK | TGFb | | | | | | TNFa | |
| 5 | 79.2% | DKK3 | M2PK | TGFb | | | | | | TNFa | |
| 5 | 78.5% | DKK3 | M2PK | TGFb | | TIMP1 | | | | | |
| 5 | 78.5% | DKK3 | M2PK | | | TIMP1 | BDNF | | | | |
| 5 | 78.3% | DKK3 | M2PK | | | | BDNF | | | TNFa | |
| 5 | 78.3% | DKK3 | M2PK | TGFb | | | | | | TNFa | |
| 5 | 78.3% | DKK3 | M2PK | TGFb | | | | | | TNFa | |
| 5 | 77.6% | DKK3 | M2PK | TGFb | | TIMP1 | | | | | IGFII |
| 5 | 77.6% | DKK3 | M2PK | | IGFBP2 | TIMP1 | BDNF | | | | |
| 5 | 77.6% | DKK3 | M2PK | TGFb | | | | | | | |
| 5 | 77.6% | DKK3 | M2PK | TGFb | | TIMP1 | | | | | |
| 5 | 77.6% | DKK3 | M2PK | | | | BDNF | | IL8 | | IGFII |
| 5 | 77.4% | DKK3 | M2PK | TGFb | | TIMP1 | | | | | |
| 5 | 77.4% | DKK3 | M2PK | TGFb | | TIMP1 | | | | TNFa | |
| 5 | 77.4% | DKK3 | M2PK | TGFb | | | | | | TNFa | |
| 5 | 77.4% | DKK3 | M2PK | TGFb | | | | | | TNFa | IGFII |
| 5 | 76.6% | DKK3 | M2PK | TGFb | | TIMP1 | | | | | |
| 5 | 76.6% | DKK3 | M2PK | TGFb | | TIMP1 | | | | | |
| 5 | 76.6% | | M2PK | | | | BDNF | | IL8 | TNFa | |
| 4 | 78.3% | DKK3 | M2PK | TGFb | | | | | | TNFa | |
| 4 | 77.6% | DKK3 | M2PK | TGFb | | TIMP1 | | | | | |
| 4 | 76.6% | DKK3 | M2PK | TGFb | | | | | | | |
| 4 | 76.6% | DKK3 | M2PK | TGFb | | | | | | | |
| 4 | 76.6% | DKK3 | M2PK | TGFb | | | | | | | |
| 4 | 76.6% | DKK3 | M2PK | | | TIMP1 | BDNF | | | | |
| 4 | 74.8% | DKK3 | M2PK | TGFb | | | | | | | |
| 4 | 74.5% | DKK3 | M2PK | TGFb | | | | | IL8 | | |
| 4 | 74.5% | DKK3 | M2PK | TGFb | | | | | | | |
| 4 | 73.8% | | M2PK | | | | BDNF | | IL8 | TNFa | |
| 4 | 73.8% | DKK3 | M2PK | TGFb | | | | | | | IGFII |
| 4 | 73.8% | DKK3 | M2PK | TGFb | IGFBP2 | | | | | | |
| 4 | 73.1% | DKK3 | M2PK | | IGFBP2 | | | | | | IGFII |
| 4 | 72.9% | DKK3 | M2PK | TGFb | | | | | | | |
| 4 | 72.9% | DKK3 | M2PK | | IGFBP2 | | | | | | |
| 4 | 72.0% | | M2PK | | IGFBP2 | | BDNF | | | TNFa | |
| 4 | 72.0% | DKK3 | M2PK | | | | | | | | |
| 4 | 72.0% | DKK3 | M2PK | | | | | | | | |
| 4 | 72.0% | | M2PK | | IGFBP2 | | BDNF | | IL8 | | |
| 4 | 71.0% | DKK3 | M2PK | | | | BDNF | | | TNFa | |
| 3 | 76.6% | DKK3 | M2PK | TGFb | | | | | | | |
| 3 | 70.1% | DKK3 | M2PK | | | | | | | | |
| 3 | 69.2% | | M2PK | | | | BDNF | | | TNFa | |
| 3 | 69.2% | | M2PK | | | TIMP1 | BDNF | | | | |
| 3 | 68.5% | DKK3 | M2PK | | | | | | | | IGFII |
| 3 | 68.5% | DKK3 | M2PK | | | | | | | | |
| 3 | 68.5% | DKK3 | M2PK | | | | | | | | |
| 3 | 68.2% | | M2PK | | | | BDNF | | IL8 | | |
| 3 | 67.6% | DKK3 | M2PK | | IGFBP2 | | | | | | |
| 3 | 67.6% | DKK3 | M2PK | | | TIMP1 | | | | | |
| 3 | 67.3% | | M2PK | TGFb | | TIMP1 | | | | | |
| 3 | 66.7% | DKK3 | M2PK | | | | | | | | |
| 3 | 65.7% | DKK3 | M2PK | | | | | | | | |
| 3 | 65.7% | DKK3 | M2PK | | | | | | | | |
| 3 | 65.4% | DKK3 | M2PK | | | | | | | | |
| 3 | 65.4% | | M2PK | TGFb | | | | | | | |
| 3 | 64.8% | | M2PK | | IGFBP2 | | | | | | IGFII |
| 3 | 64.5% | DKK3 | M2PK | | | | | | | TNFa | |
| 3 | 63.6% | | M2PK | | | | | | | TNFa | IGFII |
| 3 | 63.6% | | M2PK | | | | | IL6 | | | IGFII |

| # | Lipocalin | M30 | M65 | Mac2BP | MMP1 | MMP7 | MIP1B | Age | IL13 |
|---|---|---|---|---|---|---|---|---|---|
| 7 | | | | | | | MIP1B | | |
| 7 | | | | | MMP1 | | MIP1B | | |
| 7 | Lipocalin | | | | | | | | |
| 7 | | | | | MMP1 | MMP7 | | | |
| 7 | | | | | | MMP7 | | | |
| 7 | | | M65 | | | | MIP1B | | |
| 7 | | | | | | MMP7 | | | |
| 7 | | M30 | M65 | | | | | | |
| 7 | | M30 | | | | | | | |
| 7 | | M30 | | | | MMP7 | | | |
| 7 | | | | | | MMP7 | MIP1B | | |
| 7 | | M30 | M65 | | | | MIP1B | | |
| 7 | | | | | | MMP7 | | | IL13 |
| 7 | Lipocalin | | M65 | | | | | | |
| 7 | | | | | | | | | |
| 7 | Lipocalin | | M65 | | | | MIP1B | | |
| 7 | | | M65 | | | MMP7 | | | |
| 7 | | | | | | | MIP1B | | |
| 7 | | | | | | | MIP1B | | |
| 7 | | | | Mac2BP | | | | | |
| 6 | | | | | | | | | |
| 6 | | | | | MMP1 | | | | |
| 6 | | M30 | | | | | | | |
| 6 | | | | | | MMP7 | | | |
| 6 | | | M65 | | | | MIP1B | | |
| 6 | | | M65 | | | | | | |
| 6 | | | | | | | | | |
| 6 | Lipocalin | | | | | | | | |
| 6 | | M30 | M65 | | | | | | |
| 6 | | | | | | | | | IL13 |
| 6 | | | | | | | | | |
| 6 | | | M65 | | | | | | |
| 6 | | | | | | | MIP1B | | IL13 |
| 6 | Lipocalin | | | | | | MIP1B | | |
| 6 | Lipocalin | M30 | | | | | | | |
| 6 | | | M65 | | | | | | |
| 6 | | | | | | | MIP1B | | |
| 6 | | | | | | | MIP1B | | |
| 6 | | | | | MMP1 | | | | |
| 6 | | | | | MMP1 | | | | |
| 5 | | | | | | | | | |
| 5 | | | | | | | MIP1B | | |
| 5 | | M30 | | | | | | | |
| 5 | Lipocalin | | | | | | | | |
| 5 | Lipocalin | | | | | | | | |
| 5 | | | M65 | | | | | | |
| 5 | | | | | | | | | IL13 |
| 5 | Lipocalin | | | | | | | | |
| 5 | | | | | | | | | |
| 5 | | | | | | | | | |
| 5 | | | | | | | MIP1B | | IL13 |
| 5 | | | | | | | MIP1B | | |
| 5 | | | | | | | | | |
| 5 | | | | | | MMP7 | | | |
| 5 | | | | | | | | | |
| 5 | | | | | MMP1 | | | | |
| 5 | | | | | | | | | |
| 5 | | | | | | | | | IL13 |
| 5 | | M30 | | | | | | | |
| 5 | | | | | | | | | IL13 |
| 4 | | | | | | | | | |
| 4 | | | | | | | | | |
| 4 | | | | | | | | | IL13 |
| 4 | | | | | | | MIP1B | | |
| 4 | Lipocalin | | | | | | | | |
| 4 | | | | | | | | | |
| 4 | | | | | MMP1 | | | | |
| 4 | | | | | | | | | |
| 4 | | | | | | MMP7 | | | |
| 4 | | | | | | | | | |
| 4 | | | | | | | | | |
| 4 | | | | | | | | | |
| 4 | | | | | | | | | |
| 4 | | M30 | | | | | | | |
| 4 | | | | | | MMP7 | | | |
| 4 | | | | | | | | | |
| 4 | | | | | | MMP7 | | | IL13 |
| 4 | Lipocalin | | | | | MMP7 | | | |
| 4 | | | | | | | | | |
| 4 | | | | | | | | | |
| 3 | | | | | | | | | |
| 3 | | | | | | MMP7 | | | |
| 3 | | | | | | | | | |
| 3 | | | | | | | | | |
| 3 | | | | | | | | | |
| 3 | | | | | | | | | IL13 |
| 3 | | | | | | | MIP1B | | |
| 3 | | | | | | | | | |
| 3 | | | | | | | | | |
| 3 | | | | | | | | | |
| 3 | | | | | | | | | |
| 3 | Lipocalin | | | | | | | | |
| 3 | | M30 | | | | | | | |
| 3 | | | | | MMP1 | | | | |
| 3 | | | M65 | | | | | | |
| 3 | | | | | | | | | IL13 |
| 3 | | | | | | | | | |
| 3 | | | | | | | | | |
| 3 | | | | | | | | | |
| 3 | | | | | | | | | |

TABLE 66
Top 20, 7 to 3 biomarker combinations which may be used to differentiate colorectal cancer
subjects (male only, age also included in analysis) from controls at 95% specificity.

| # | Sens. | Dkk3 | M2PK | TGFb | IGFBP2 | TIMP1 | BDNF | IL6 | IL8 | TNFa | IGFII |
|---|---|---|---|---|---|---|---|---|---|---|---|
| 7 | 82.1% | DKK3 | M2PK | | IGFBP2 | | BDNF | | | TNFa | |
| 7 | 81.7% | DKK3 | M2PK | TGFb | IGFBP2 | | | | | TNFa | |
| 7 | 81.3% | DKK3 | M2PK | | | TIMP1 | BDNF | | IL8 | TNFa | |
| 7 | 81.3% | DKK3 | M2PK | | | TIMP1 | BDNF | | IL8 | TNFa | |
| 7 | 81.1% | DKK3 | M2PK | | | | BDNF | | | TNFa | |
| 7 | 81.1% | DKK3 | M2PK | | | | BDNF | | | TNFa | |
| 7 | 81.1% | DKK3 | M2PK | | | TIMP1 | BDNF | | IL8 | TNFa | |
| 7 | 81.0% | DKK3 | M2PK | | IGFBP2 | | BDNF | | | TNFa | |
| 7 | 80.4% | DKK3 | M2PK | | IGFBP2 | | BDNF | | IL8 | TNFa | |
| 7 | 80.4% | DKK3 | M2PK | | IGFBP2 | | BDNF | | IL8 | TNFa | |
| 7 | 80.2% | DKK3 | M2PK | | IGFBP2 | | BDNF | | | TNFa | |
| 7 | 80.2% | DKK3 | M2PK | TGFb | IGFBP2 | | BDNF | | IL8 | TNFa | |
| 7 | 80.2% | DKK3 | M2PK | TGFb | | TIMP1 | BDNF | | IL8 | TNFa | |
| 7 | 80.2% | DKK3 | M2PK | | | TIMP1 | BDNF | | | | IGFII |
| 7 | 80.2% | DKK3 | M2PK | | | | BDNF | | | TNFa | |
| 7 | 80.2% | DKK3 | M2PK | | | TIMP1 | BDNF | | | | |
| 7 | 80.0% | DKK3 | M2PK | TGFb | | | BDNF | | | TNFa | |
| 7 | 80.0% | DKK3 | M2PK | | | | BDNF | | | TNFa | |
| 7 | 80.0% | DKK3 | M2PK | TGFb | IGFBP2 | | | IL6 | | | |
| 7 | 79.4% | DKK3 | M2PK | | IGFBP2 | | BDNF | | IL8 | TNFa | IGFII |
| 6 | 81.3% | DKK3 | M2PK | | | TIMP1 | BDNF | | IL8 | TNFa | |
| 6 | 81.3% | DKK3 | M2PK | | IGFBP2 | | BDNF | | IL8 | TNFa | |
| 6 | 81.1% | DKK3 | M2PK | | | | BDNF | | | TNFa | |
| 6 | 80.2% | DKK3 | M2PK | TGFb | IGFBP2 | | | | | | |
| 6 | 80.2% | DKK3 | M2PK | | | | BDNF | | | TNFa | |
| 6 | 80.2% | DKK3 | M2PK | | | TIMP1 | BDNF | | | | |
| 6 | 80.0% | DKK3 | M2PK | TGFb | IGFBP2 | | | | | | |
| 6 | 79.4% | DKK3 | M2PK | | | TIMP1 | BDNF | | IL8 | | |
| 6 | 79.4% | DKK3 | M2PK | | IGFBP2 | | BDNF | | | TNFa | |
| 6 | 79.2% | DKK3 | M2PK | | IGFBP2 | | BDNF | | | TNFa | |
| 6 | 79.2% | DKK3 | M2PK | | IGFBP2 | | BDNF | | | TNFa | |
| 6 | 79.2% | DKK3 | M2PK | | | TIMP1 | BDNF | | | | |
| 6 | 79.2% | DKK3 | M2PK | TGFb | | | | | | TNFa | |
| 6 | 79.2% | DKK3 | M2PK | TGFb | | | | | | TNFa | |
| 6 | 79.0% | DKK3 | M2PK | TGFb | IGFBP2 | | | IL6 | | | |
| 6 | 79.0% | DKK3 | M2PK | TGFb | IGFBP2 | | | | | TNFa | |
| 6 | 79.0% | DKK3 | M2PK | TGFb | IGFBP2 | | | | | TNFa | |
| 6 | 79.0% | DKK3 | M2PK | | | | BDNF | | | TNFa | |
| 6 | 78.5% | DKK3 | M2PK | | IGFBP2 | | BDNF | | | TNFa | IGFII |
| 6 | 78.5% | DKK3 | M2PK | | IGFBP2 | | BDNF | | | | |
| 5 | 79.2% | DKK3 | M2PK | | | | BDNF | | | TNFa | |
| 5 | 79.2% | DKK3 | M2PK | TGFb | | | | | | TNFa | |
| 5 | 78.5% | DKK3 | M2PK | TGFb | | TIMP1 | | | | | |
| 5 | 78.5% | DKK3 | M2PK | TGFb | | TIMP1 | | | | | |
| 5 | 78.5% | DKK3 | M2PK | TGFb | | TIMP1 | | | | | |
| 5 | 78.3% | DKK3 | M2PK | TGFb | IGFBP2 | | | | | | |
| 5 | 78.3% | DKK3 | M2PK | TGFb | | TIMP1 | | | | | |
| 5 | 78.3% | DKK3 | M2PK | TGFb | | | | | | TNFa | |
| 5 | 78.3% | DKK3 | M2PK | TGFb | | TIMP1 | | | | TNFa | |
| 5 | 78.3% | DKK3 | M2PK | TGFb | | | | | | TNFa | |
| 5 | 77.6% | DKK3 | M2PK | TGFb | | TIMP1 | | | | | IGFII |
| 5 | 77.6% | DKK3 | M2PK | TGFb | | | | | | | |
| 5 | 77.4% | DKK3 | M2PK | | | TIMP1 | BDNF | | | | |
| 5 | 77.4% | DKK3 | M2PK | TGFb | | | | | | TNFa | |
| 5 | 77.1% | DKK3 | M2PK | | | | BDNF | | | | |
| 5 | 76.6% | DKK3 | M2PK | | IGFBP2 | | BDNF | | | TNFa | |
| 5 | 76.6% | DKK3 | M2PK | TGFb | | | | | | | |
| 5 | 76.6% | DKK3 | M2PK | TGFb | | TIMP1 | | | | | |
| 5 | 76.6% | DKK3 | M2PK | TGFb | | | | | | | |
| 5 | 76.6% | | M2PK | | | | BDNF | | IL8 | TNFa | |
| 4 | 78.5% | DKK3 | M2PK | TGFb | | TIMP1 | | | | | |
| 4 | 78.3% | DKK3 | M2PK | TGFb | | | | | | TNFa | |
| 4 | 77.6% | DKK3 | M2PK | TGFb | | | | | | | |
| 4 | 76.6% | DKK3 | M2PK | TGFb | | | | | | | |
| 4 | 75.7% | DKK3 | M2PK | TGFb | | | | | | | |
| 4 | 75.7% | | M2PK | | IGFBP2 | | BDNF | | | TNFa | |
| 4 | 75.7% | DKK3 | M2PK | | | TIMP1 | BDNF | | | | |
| 4 | 74.8% | | M2PK | | | | BDNF | | IL8 | TNFa | |
| 4 | 74.5% | DKK3 | M2PK | TGFb | | | | | | | |
| 4 | 73.8% | DKK3 | M2PK | TGFb | | | | | | | IGFII |
| 4 | 72.9% | DKK3 | M2PK | TGFb | IGFBP2 | | | | | | |
| 4 | 72.9% | DKK3 | M2PK | TGFb | | | | | | | |
| 4 | 72.9% | DKK3 | M2PK | TGFb | | | | | | | |
| 4 | 72.6% | DKK3 | M2PK | TGFb | | | | | IL8 | | |
| 4 | 72.2% | DKK3 | M2PK | | IGFBP2 | | | | | | IGFII |
| 4 | 72.0% | DKK3 | M2PK | | | | BDNF | | IL8 | | |
| 4 | 72.0% | DKK3 | M2PK | | | | | | | | |
| 4 | 71.0% | DKK3 | M2PK | | | | BDNF | | | TNFa | |
| 4 | 71.0% | DKK3 | M2PK | | | | | | | TNFa | IGFII |
| 4 | 71.0% | | M2PK | | | TIMP1 | BDNF | | IL8 | | |
| 3 | 76.6% | DKK3 | M2PK | TGFb | | | | | | | |
| 3 | 68.5% | DKK3 | M2PK | | IGFBP2 | | | | | | |
| 3 | 68.2% | | M2PK | | | | BDNF | | IL8 | | |
| 3 | 67.6% | DKK3 | M2PK | | | | | | | | IGFII |
| 3 | 67.6% | DKK3 | M2PK | | | TIMP1 | | | | | |
| 3 | 67.3% | DKK3 | M2PK | | | | | | | | |
| 3 | 66.7% | DKK3 | M2PK | | | | | | | | |
| 3 | 66.7% | DKK3 | M2PK | | | | | | | | |
| 3 | 66.7% | DKK3 | M2PK | | | | | | | | |
| 3 | 66.7% | DKK3 | M2PK | | | | | | | | |
| 3 | 65.7% | | M2PK | | IGFBP2 | | | | | | IGFII |
| 3 | 65.4% | | M2PK | | | | | | | | |
| 3 | 65.4% | DKK3 | M2PK | | | | | | | | |
| 3 | 65.1% | | M2PK | | | | | | | TNFa | |
| 3 | 64.8% | DKK3 | M2PK | | | | | | | | |
| 3 | 64.2% | | M2PK | | | | BDNF | | | | |
| 3 | 63.6% | | M2PK | | | | BDNF | | | TNFa | |
| 3 | 63.6% | | M2PK | TGFb | IGFBP2 | | | | | | |
| 3 | 63.6% | | M2PK | | | | | | | | |
| 3 | 63.2% | | M2PK | | | | | | | | |

| # | Lipocalin | M30 | M65 | Mac2BP | MMP1 | MMP7 | MIP1B | Age | IL13 |
|---|---|---|---|---|---|---|---|---|---|
| 7 | | M30 | | | | MMP7 | | Age | |
| 7 | | | M65 | | | MMP7 | | Age | |
| 7 | | | | | | | MIP1B | Age | |
| 7 | Lipocalin | | | | | | | Age | |
| 7 | | | M65 | | | | MIP1B | Age | IL13 |
| 7 | | M30 | M65 | | | | MIP1B | Age | |
| 7 | | | | | | MMP7 | | Age | |
| 7 | | | M65 | | | MMP7 | | Age | |
| 7 | | M30 | | | | | | Age | |
| 7 | | | | | MMP1 | | | Age | |
| 7 | | | M65 | | | | MIP1B | Age | |
| 7 | | | | | | | | Age | |
| 7 | | | | | | | | Age | |
| 7 | Lipocalin | | | | | MMP7 | | Age | |
| 7 | Lipocalin | | M65 | | | | MIP1B | Age | |
| 7 | Lipocalin | M30 | | | | MMP7 | | Age | |
| 7 | | | M65 | | | | MIP1B | Age | |
| 7 | | | M65 | | | MMP7 | MIP1B | Age | |
| 7 | | | | | | MMP7 | MIP1B | Age | |
| 7 | | | | | | | | Age | |
| 6 | | | | | | | | Age | |
| 6 | | | | | | | | Age | |
| 6 | | | M65 | | | | MIP1B | Age | |
| 6 | | | | | MMP1 | MMP7 | | Age | |
| 6 | | M30 | M65 | | | | | Age | |
| 6 | | M30 | | | | MMP7 | | Age | |
| 6 | | | M65 | | | MMP7 | | Age | |
| 6 | | | | | MMP1 | | | Age | |
| 6 | | M30 | | | | | | Age | |
| 6 | | | | | | MMP7 | | Age | |
| 6 | | | M65 | | | | | Age | |
| 6 | Lipocalin | | | | | MMP7 | | Age | |
| 6 | | | | | | | MIP1B | Age | IL13 |
| 6 | Lipocalin | | | | | | MIP1B | Age | |
| 6 | | | | | | MMP7 | | Age | |
| 6 | | | | | | MMP7 | | Age | |
| 6 | | | M65 | | | | | Age | |
| 6 | | | M65 | | | MMP7 | | Age | |
| 6 | | | | | | | | Age | |
| 6 | | | | Mac2BP | MMP1 | | | Age | |
| 5 | | | M65 | | | | | Age | |
| 5 | | | | | | | MIP1B | Age | |
| 5 | | | | | | | MIP1B | Age | |
| 5 | | | | | | | | Age | IL13 |
| 5 | Lipocalin | | | | | | | Age | |
| 5 | | | | | | MMP7 | | Age | |
| 5 | | | | | | MMP7 | | Age | |
| 5 | | | | | | | | Age | IL13 |
| 5 | | | | | | | | Age | |
| 5 | Lipocalin | | | | | | | Age | |
| 5 | | | | | | | | Age | |
| 5 | | | | | | | MIP1B | Age | IL13 |
| 5 | | | | | | MMP7 | | Age | |
| 5 | | | | | MMP1 | | | Age | |
| 5 | | | M65 | | | MMP7 | | Age | |
| 5 | | | | | | | | Age | |
| 5 | Lipocalin | | | | | | | Age | IL13 |
| 5 | | M30 | | | | | | Age | |
| 5 | Lipocalin | | | | | | MIP1B | Age | |
| 5 | | | | | | | | Age | IL13 |
| 4 | | | | | | | | Age | |
| 4 | | | | | | | | Age | |
| 4 | | | | | | | MIP1B | Age | |
| 4 | | | | | | | | Age | IL13 |
| 4 | Lipocalin | | | | | | | Age | |
| 4 | | | | | | | | Age | |
| 4 | | | | | | | | Age | |
| 4 | | | | | | | | Age | |
| 4 | | | | | | MMP7 | | Age | |
| 4 | | | | | | | | Age | |
| 4 | | | | | | | | Age | |
| 4 | | | | | MMP1 | | | Age | |
| 4 | | M30 | | | | | | Age | |
| 4 | | | | | | | | Age | |
| 4 | | | | | | | | Age | |
| 4 | | | | | | | | Age | |
| 4 | | | | | | MMP7 | | Age | IL13 |
| 4 | | | | | | | | Age | |
| 4 | | | | | | | | Age | |
| 4 | | | | | | | | Age | |
| 3 | | | | | | | | Age | |
| 3 | | | | | | | | Age | |
| 3 | | | | | | | | Age | |
| 3 | | | | | | | | Age | |
| 3 | | | | | | | | Age | |
| 3 | | | | | | MMP7 | | Age | |
| 3 | | | | | | | MIP1B | Age | |
| 3 | | | | | | | | Age | IL13 |
| 3 | | | | | MMP1 | | | Age | |
| 3 | Lipocalin | | | | | | | Age | |
| 3 | | | | | | | | Age | |
| 3 | | | | | MMP1 | MMP7 | | Age | |
| 3 | | | M65 | | | | | Age | |
| 3 | | | | | | MMP7 | | Age | |
| 3 | | M30 | | | | | | Age | |
| 3 | | | | | | MMP7 | | Age | |
| 3 | | | | | | | | Age | |
| 3 | | | | | | | | Age | |
| 3 | | | | | | MMP7 | | Age | IL13 |
| 3 | | | M65 | | | MMP7 | | Age | |

### Example 6 Performance of a Five Biomarker Panel (Study 9)

To examine the potential utility of a blood-based five-biomarker panel for the early detection of CRC and to examine its potential application to CRC screening of asymptomatic persons at normal-risk for developing CRC, a case/control study was first performed to identify the performance characteristics of an example five biomarker panel comprising M2PK, BDNF, IGFBP2, TIMP1 and DKK3.

Such a panel could be useful in a number of contexts: As an adjunct to current FIT or colonoscopy screening, providing an alternative test for people who cannot or will not test for colorectal cancer using a stool test; as an additional test to facilitate triage of persons with a positive FIT result for colonoscopy or potentially as an alternative to FIT for first-line CRC screening applications. Comparison of the performance of the five-biomarker panel to that of FIT is therefore important, particularly the relative positive and negative predictive values of the tests when applied to an asymptomatic, normal-risk, screening population.

### Methods

### Study Populations

TABLE 67
Study populations of cohort 1 and cohort 2.

| | Cohort 1 | | Cohort 2 | |
|---|---|---|---|---|
| Characteristics | Control | CRC | Control | CRC |
| N | 167 | 182 | 45 | 49 |
| Gender, N | | | | |
| Female | 85 | 85 | 21 | 25 |
| Male | 82 | 97 | 24 | 24 |
| Median BMI (range) | 29 (15-59) | 26 (18-44) | 27 (19-39) | 27 (20-49) |
| Median age, yrs. | 62 (39-86) | 62 (40-81) | 64 (42-81) | 65 (40-80) |
| (range) | | | | |
| AJCC TNM stage | | | | |
| I | N/A | 46* | N/A | 9 |
| II | N/A | 55 | N/A | 23 |
| III | N/A | 53 | N/A | 17 |
| IV | N/A | 25 | N/A | 0 |

CRC = colorectal cancer
Cohort 1 comprised of 349 samples and Cohort 2 comprised 94 samples.
*Three samples were Stage 0 cancer and are not included in TMN stage counts.

### Samples and Quantification

Serum samples for this study, collected between 2018 and 2021, were sourced from the Victorian Cancer Biobank or commercially from ProteoGenex (ProteoGenex, Inc., Inglewood, CA, USA). Research protocols were approved by the Cancer Council Victoria Human Research Ethics Committee (HREC-1803) and the Russian Oncological Research Center Ethics Committee (IRB PG-ONC 2003/1) via ProteoGenex.

The concentration of M2PK, BDNF, IGFBP2, TIMP1 and DKK3 were quantified in serum samples from two independent CRC case/healthy normal control cohorts whose characteristics are described in Table 67. ELISA was used to measure the concentration of the biomarkers. The biomarker concentrations were combined with age, gender or BMI data via the algorithm described herein to provide a colorectal cancer likelihood score. In the present example, females were assigned an arbitrary value of 1.1 and males were assigned an arbitrary value of 1. It is anticipated that persons with a score above a defined threshold will be advised by their healthcare professional to progress to colonoscopy for a definitive diagnosis. Those with scores below the threshold will be advised to screen again in two years' time.

### Biomarker Panel Training and Testing

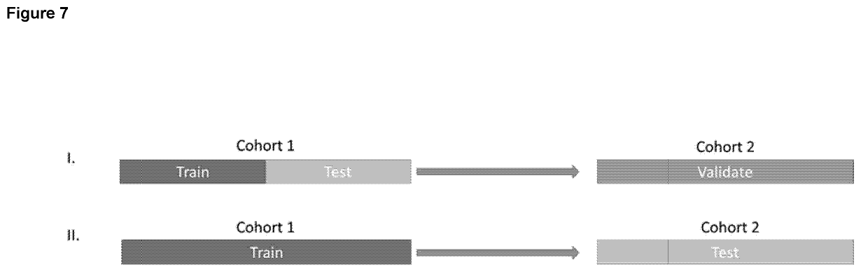

Sample concentrations from both cohorts, with the addition of age and gender, were used in the training and testing of logistic regression-based algorithms. In deriving these equations, protein biomarker concentrations were log 10 transformed. Femaleness was allocated a biomarker value of 1.0, maleness, a value of 1.1. Age was expressed in years. Body Mass Index values were as calculated from participants height and weight measurements in deriving those algorithms where BMI was included. Train-test split (with split ratios of 60:40, 70:30, 80:20, 90:10, and 100:100) and K-fold cross validation (K=5, 10) methods on shuffled data, with 100 resamples and 1000 iterations, were applied to generate algorithms comprising the five to eight-parameter panels under consideration. Train, test and validate methods were applied in two different approaches (FIG. 7).
One cohort was split using train-test split or k-fold cross validation to create a train and test subset. The resultant algorithm was then validated on the second cohort.
One cohort in its entirety was used to train an algorithm which was then tested on the second cohort.

The python-based integrated development environment PyCharm (Version X, JetBrains, Prague 4, Prague, Czech Republic) and the numeric computing environment MATLAB (Version 2021b, MathWorks, Natick, MA, USA) were used to perform logistic regression and fuzzy logic analysis on the data.

### Algorithm Selection

Algorithms trained and in-sample tested, on cohort 1, were tested on cohort 2. The top performing algorithms were chosen based on performance (training and testing sensitivity above a performance target of 73% at a specificity of 95%), confidence in performance, robustness (transferability between datasets) and training dataset size. The Wilson score interval with 95% confidence was calculated manually for top performing algorithm sensitivities with the number of true positives (sensitivity) represented as a binomial distribution ((E. B. Wilson, “Probable inference, the law of succession, and statistical inference,” Journal of the American Statistical Association, vol. 22, no. 158, pp. 209-212, 1927). The 73% performance target was chosen based on a meta-analysis of FITS using a cut-off value of 20 ug Hb/g (K. Selby et al., “Effect of sex, age, and positivity threshold on fecal immunochemical test accuracy: a systematic review and meta-analysis,” Gastroenterology, vol. 157, no. 6, pp. 1494-1505, 2019). Results for the top performing algorithms are shown below.

### Results

### Performance of Individual Biomarkers Measured in the Serum of Colorectal Cancer (CRC) and Control Patients

Considered individually, the levels of each of the five biomarkers differed significantly between the CRC and control serum samples in Cohort 1 (Table 68). Levels of M2PK, IGFBP2 and TIMP1 were elevated in sera from CRC patients relative to controls while levels of BDNF and DKK3 were reduced relative to controls. In the smaller Cohort 2, significant differences in biomarker concentrations between cancer and control sera were observed for M2PK, IGFBP2 and TIMP1 while DKK3 approached significance.

TABLE 68
Concentration (median and range) for individual protein biomarkers measured in the serum of cancer
and control patients. P values determined using the non-parametric Wilcoxon rank sum test.

| | Cohort 1 | | | Cohort 2 | | |
|---|---|---|---|---|---|---|
| | Control | CRC | P Value | Control | CRC | P Value |
| M2PK | 497.41 | 1216.66 | <0.0001 | 411.24 | 1125.14 | <0.0001 |
| (U/ml) | (66.86-337.50) | (130.02-3357.50) | | (115.36-1323.37) | (198.23-3357.50) | |
| DKK3 | 37.19 | 33.99 | 0.0049 | 35.49 | 32.58 | 0.0571 |
| (ng/ml) | (19.00-104.92) | (12.44-89.485) | | (21.555-55.44) | (18.23-59.85) | |
| IGFBP2 | 289.17 | 544.77 | <0.0001 | 291.37 | 509.965 | <0.0001 |
| (ng/ml) | (69.36-1049.22) | (121.85-1049.22) | | (106.67-763.37) | (110.38-1049.22) | |
| TIMP1 | 310.40 | 338.08 | <0.0001 | 302.28 | 365.08 | 0.0001 |
| (ng/ml) | (123.59-586.42) | (152.66-1049.22) | | (213.38-561.36) | (167.755-876.01) | |
| BDNF | 12.015 | 10.58 | <0.0001 | 11.6075 | 10.965 | 0.3041 |
| (ng/ml) | (6.70-24.24) | (3.13-24.80) | | (7.605-16.535) | (1.99-20.025) | |

When considered individually, M2PK discriminated best between cancer and controls with minimum P values of 8.30e⁻⁰⁶ and 5.46e⁻⁰⁰⁹ in Cohorts 1 and 2 respectively. IGFBP2 was the second-best with P values of 4.68e⁻⁰²³ and 1.03e⁻⁰⁰⁶ in Cohorts 1 and 2 respectively. Discrimination between cases and controls was lowest for DKK3 in Cohort 1 (P=0.0049) while BDNF was lowest in Cohort 2 (P=0.3041) which may have been a result of the small size of this cohort. Based on ROC analysis, however, none of these markers individually differentiated between cases and controls with sufficient accuracy to be used clinically for the early detection of CRC.

### Biomarker Panel Performance

To determine whether, these five (M2PK, DKK3, IGFBP2, TIMP1 and BDNF) biomarkers could, when coupled with terms for age, gender or body mass index (BMI), usefully differentiate between samples from CRC patients and controls, the inventors applied logistic regression and Receiver Operator Characteristic (ROC) curve analysis.

High-performing algorithms, combining biomarker concentrations with additional terms for age and gender, that differentiated between case and control samples with high sensitivity and specificity were trained on data from Cohort 1 as described herein. Lead algorithms were then cross validated in-sample using multiple iterations of an 80/20 split, train/test approach. The algorithm producing the highest cross validated sensitivity and specificity on ROC analysis (point closest-to-(0, 1) corner in the ROC plane), was selected and locked. Included in these locked parameters is the threshold value above which a test result is considered positive and below which a test is scored as negative. This algorithm was then tested on the fully independent data set, (Cohort 2). For each analysis, area under the ROC curve was determined. Sensitivity and specificity values at the locked threshold value were determined, along with positive and negative predictive values.

The results were:

| AUC | Cohort 1 cross validated-88% | Cohort 2 cross validated-84% |
|---|---|---|
| Sensitivity | Cohort 1 cross validated-84% | Cohort 2 cross validated-78% |
| Specificity | Cohort 1 cross validated-97% | Cohort 2 cross validated-93% |
| PPV | Cohort 1 cross validated- | Cohort 2 cross validated- |
| | 97.1% | 92.7% |
| NPV | Cohort 1 cross validated- | Cohort 2 cross validated- |
| | 83.8% | 78.8% |

Small apparent variations in sensitivity values across these different training and test operations were not statistically significant. Further, the sensitivity and specificity values are highly competitive with the those observed for FIT in the Australian NBCSP Pilot study performed on an asymptomatic, normal CRC risk population aged 50 to 69 yrs.

Mapping the sensitivity and specificity values for the five protein plus age and gender classifier described above to a theoretical normal CRC risk screening population of one million participants exhibiting a CRC prevalence of 0.00264 (Australian Institute of Health and Welfare 2014. Analysis of bowel cancer outcomes for the National Bowel Cancer Screening Program. Cat. no. CAN 87. Canberra: AIHW) allows calculation of a theoretical positive predictive value (PPV) and negative predictive value (NPV) expected in a screening population. A comparison of these values relative to equivalents observed in FIT population screening in the Australian National Bowel Cancer Screening program is shown in Table 69.

TABLE 69
Comparison of FIT and projected ColoSTAT performance in
population screening for CRC

| | NBCSP | ColoSTAT modelling (5 |
|---|---|---|
| Parameter | Pilot (2006-2008) | biomarkers + age + gender) |
| Prevalence | 0.00264 | 0.00264 |
| of CRC | | |
| N | 595,705 | 1,000,000 |
| Sensitivity | 83.4% | 84%* |
| Specificity | 92.6% | 97%* |
| PPV | 0.036 | 0.069 |
| NPV | 0.999 | 0.9996 |

*Values determined from case/control studies.

Results in the Table 69 project a substantial improvement in performance parameters for the five-biomarker panel including BDNF, M2PK, IGFBP2, TIMP1 and DKK3, plus age and gender, over FIT suggesting strong potential utility when applied to an asymptomatic, normal CRC-risk screening population.

As would be appreciated by the person skilled in the art, while the present example was achieved with quantification of a five-protein biomarker panel (M2PK, BDNF, IGFBP2, TIMP1 and IGFBP2) plus demographic indicators for age and gender, a similar strong performance may be also be expected using the five-protein biomarkers alone, in conjunction with age only and in conjunction with gender only. Further, it will be understood by the person skilled in the art that the addition or substitution of one or more demographic terms with other demographic or morphometric terms including but not limited to smoking history, body mass index (BMI) and hip to waist ratio would also be expected to provide strong-performing tests highly competitive with FIT.

The results tables below support this understanding. They describe the sensitivity and specificity data for the 5 protein biomarkers comprising BDNF, M2PK, IGFBP2, TIMP1 and DKK3 alone as well as in conjunction with additional demographic and morphometric biomarkers including the subject's age, gender and BMI (body Mass Index values were as calculated from participants height and weight measurements). For the consideration of gender, females were assigned an arbitrary value of 1.1 and males were assigned an arbitrary value of 1.

With reference to the tables below, BM1 refers to PKM2 Tumour form; BM2=TIMP1; BM3=IGFBP2; BM4=DKK3 and BM5=BDNF.

TABLE 70
Average performance of each model.

| | | | Cohort 1 | Cohort 1 | |
|---|---|---|---|---|---|
| | | | Average | Average | Cohort 2 |
| | | | Train | Test | Average |
| | Cohort 1 | Cohort 1 | Sensitivity | Sensitivity | Sensitivity |
| | Average | Average | (%) | (%) | (%) |
| | Sensitivity | Specificity | @95% | @95% | @95% |
| | (%) [Std] | (%) [Std] | Specificity | Specificity | Specificity |
| Biomarker Panel Category | Test | Test | [Std] | [Std] | [Std] |
| BM1 + BM2 + BM3 + BM4 + BM5 | 81 [6.76] | 93 [2.27] | 73 [2.12] | 80 [8.23] | 65 [11.48] |
| BM1 + BM2 + BM3 + BM4 + BM5 + | 81 [6.18] | 93 [2.31] | 73 [4.07] | 78 [8.77] | 64 [11.18] |
| Age | | | | | |
| BM1 + BM2 + BM3 + BM4 + BM5 + | 82 [5.13] | 93 [2.92] | 73 [3.83] | 79 [9.32] | 62 [9.72] |
| Gender | | | | | |
| BM1 + BM2 + BM3 + BM4 + BM5 + | 80 [4.91] | 93 [2.38] | 73 [2.43] | 76 [8.07] | 63 [9.66] |
| Age + Gender | | | | | |
| BM1 + BM2 + BM3 + BM4 + BM5 + | 84 [6.07] | 94 [2.36] | 73 [4.24] | 83 [9.18] | 57 [10.30] |
| Age + Gender + BMI | | | | | |
| BM1 + BM2 + BM3 + BM4 + BM5 + | 85 [8.85] | 98 [3.51] | 76 [9.59] | 92 [10.53] | 73 [15.71] |
| BMI | | | | | |

[Std] represents standard deviation of the mean.

All ‘test’ outcomes consider all samples (i.e., no samples excluded). Values for sensitivity and specificity in the second and third columns have been measured at the point that minimises the Euclidean distance between the ROC curve and the (0, 1) point. Sensitivities represented in the fourth, fifth and sixth columns have been determined at thresholds resulting in 95% specificity.

### Summary

All Biomarker Panel Categories examined exhibited strong cancer/healthy control discriminating performance. Combining the 5 protein biomarkers with BMI appears to perform best with mean specificity. Statistically, however, panels comprising the 5 protein biomarkers alone, the 5 protein biomarkers markers plus age, the 5 protein biomarkers plus gender, the 5 protein biomarkers plus age plus gender, the 5 protein biomarkers plus age plus gender plus BMI and the 5 protein biomarkers plus BMI appear to be comparable.

It should be noted that impact of age and gender may have been underestimated in this particular study. Case and control samples are more closely matched in these cohorts than might be expected to occur in either prospectively recruited, clinically symptomatic patients or asymptomatic, normal-risk, CRC screening populations aged 50-74 years. Importantly, however, these results indicate that useful biomarker panels and algorithms can be developed using this five-protein biomarker panel either alone or in combination with a range of additional demogrpahic and/or morphometric parameters.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

1. A method for identifying a subject suspected of having colorectal cancer, the method comprising:
determining a measurement for a panel of biomarkers in a biological sample obtained from the subject, the panel comprising at least brain derived neurotropic factor (BDNF) and tumour M2-PK;
wherein the measurement comprises measuring a level of each of the biomarkers in the panel.

2. The method of claim 1, further comprising determining a measurement of one or more additional biomarkers selected from the group consisting of DKK3, TGFβ1, IGFBP2, TIMP1, IL6, IL8, TNFα, IGFII, Lipocalin, M30, M65, Mac2BP, MMP1, MMP7, MIP1B and IL13.

3. The method of claim 1, wherein the biomarkers comprise M2PK, BDNF, DKK3, and IGFBP2.

4. The method of claim 1, wherein the biomarkers comprise M2PK, BDNF, DKK3, TIMP1 and IGFBP2.

5. The method according to claim 1, further comprising the subject's age and/or gender and/or body mass index (BMI) as an additional biomarker.

6. The method of claim 1, comprising determining a measurement for a panel of at least 3 biomarkers, wherein the panel of biomarkers comprise or consist of:
i) BDNF, M2PK, DKK-3;
ii) BDNF, M2PK, TNFα;
iii) BDNF, M2PK, IL-8;
iv) BDNF, M2PK, MAC2BP; or
v) BDNF, M2PK, IGFBP2.

7. The method of claim 1, comprising determining a measurement for a panel of at least four biomarkers, wherein the panel of biomarkers comprise or consist of:
i) BDNF, M2PK, IL8, TNFα;
ii) BDNF, M2PK, IL8, MIP1β;
iii) BDNF, M2PK, IL8, DKK3;
iv) BDNF, M2PK, MMP1, DKK3;
v) BDNF, M2PK, IL8, IGFBP2;
vi) BDNF, M2PK, IL8, TIMP1;
vii) BDNF, M2PK, MMP7, TNFα;
viii) BDNF, M2PK, IL8, MMP1;
ix) BDNF, M2PK, IGFBP2, TNFα; or
x) BDNF, M2PK, IL8, IGFII.

8. The method of claim 1, wherein the method comprises determining a measurement for a panel of at least four biomarkers, wherein the panel comprises at least BDNF, M2PK, IL-8 and a further biomarker selected from the group consisting of DKK3, TNFα, IGFBP2, TIMP1, MIP1β, MMP7, MMP1 and IGFII.

9. The method of claim 1, comprising determining a measurement for a panel of at least five biomarkers, wherein the panel of biomarkers comprise or consist of:
i) BDNF; M2PK; IL8; TNFα; MIP1β;
ii) BDNF; M2PK; IL8; LIPOCALIN; DKK3;
iii) BDNF; M2PK; IL8; TGFβ; DKK3;
iv) BDNF; M2PK; IL8; TNFα; TGFβ;
v) BDNF; M2PK; IGFBP2; TNFα; DKK3;
vi) BDNF; M2PK; IL8; MMP7; DKK3;
vii) BDNF; M2PK; IL8; TNFα; DKK3;
viii) BDNF; M2PK; IL8; IGFBP2; MIP1β;
ix) BDNF; M2PK; IL8; MAC2BP; DKK3; or
x) BDNF; M2PK; IL8; TNFα; MAC2BP.

10. The method of claim 1, comprising determining a measurement for a panel of at least six biomarkers, wherein the panel of biomarkers comprise or consist of:
i) BDNF; M2PK; MIP1β; DKK3; IGFBP2; TNFα;
ii) BDNF; M2PK; MIP1β; DKK3; IGFBP2; MMP1;
iii) BDNF; M2PK; MAC2BP; DKK3; IGFBP2; TNFα;
iv) BDNF; M2PK; LIPOCALIN; DKK3; IGFBP2; TNFα;
v) BDNF; M2PK; IL6; DKK3; IGFBP2; TNFα;
vi) BDNF; M2PK; IL8; DKK3; LIPOCALIN; TGFβ;
vii) BDNF; M2PK; IL8; MIP1B; TGFβ; TNFα;
viii) BDNF; M2PK; IL8; MIP1B; IGFII; TNFα;
ix) BDNF; M2PK; IL8; M30; TGFβ; TNFα; or
x) BDNF; M2PK; IL8; DKK3; IGFII; TNFα.

11. The method of claim 2, wherein the panel further comprises the subject's age as an additional biomarker.

12-31. (canceled)

32. The method of claim 1, wherein determining a measurement comprises contacting the biological sample with detectable binding agents that specifically bind to each of the biomarkers.

33. The method of claim 1, wherein the method comprises detecting specific binding between the specific binding agents and the biomarkers using a detection assay.

34. The method according to claim 32, wherein determining a measurement comprises measuring the concentration of biomarker in the biological sample.

35. The method of claim 32, wherein the binding agent is an antibody.

36. The method of claim 1, wherein the biological sample is a blood sample.

37. A method of treating a subject suspected of having colorectal cancer, the method comprising:
(i) determining a measurement for a panel of biomarkers in a biological sample obtained from the subject according to claim 1; and
(ii) treating the subject by colonoscopy or sigmoidoscopy.

38. A kit comprising:
(i) labelled antibodies that bind to the biomarkers in a biomarker panel defined in claim 1; and
(ii) instructions for performing detection of the biomarkers in the biomarker panel.

39. The kit of claim 38, further comprising instructions for the analysis of the detected biomarkers by a computer generated algorithm.

40. (canceled)


SEQUENCE LISTING (21 sequences)

SEQ ID NO: 1 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 2 - protein, 328 aa, Homo sapiens
MLPRVGCPALPLPPPPLLPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEARGVHTQRMQ

SEQ ID NO: 3 - protein, 585 aa, Homo sapiens
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 4 - protein, 531 aa, Homo sapiens
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 5 - protein, 531 aa, Homo sapiens
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAMFHRKLFEELVRASSHSTDLMEAMAMGSVEASYKCLAAALIVLTESGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 6 - protein, 531 aa, Homo sapiens; identical to SEQ ID NO: 5

SEQ ID NO: 7 - protein, 146 aa, Homo sapiens
MHPLLNPLLLALGLMALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 8 - protein, 350 aa, Homo sapiens
MQRLGATLLCLLLAAAVPTAPAPAPTATSAPVKPGPALSYPQEEATLNEMFREVEELMEDTQHKLRSAVEEMEAEEAAAKASSEVNLANLPPSYHNETNTDTKVGNNTIHVHREIHKITNNQTGQMVFSETVITSVGDEEGRRSHECIIDEDCGPSMYCQFASFQYTCQPCRGQRMLCTRDSECCGDQLCVWGHCTKMATRGSNGTICDNQRDCQPGLCCAFQRGLLFPVCTPLPVEGELCHDPASRLLDLITWELEPDGALDRCPCASGLLCQPHSHSLVYVCKPTFVGSRDQDGEILLPREVPDEYEVGSFMEEVRQELEDLERSLTEEMALREPAAAAAALLGGEEI

SEQ ID NO: 9 - protein, 350 aa, Homo sapiens; identical to SEQ ID NO: 8

SEQ ID NO: 10 - protein, 350 aa, Homo sapiens; identical to SEQ ID NO: 8

SEQ ID NO: 11 - protein, 92 aa, Homo sapiens
MKLCVTVLSLLMLVAAFCSPALSAPMGSDPPTACCFSYTARKLPRNFVVDYYETSSLCSQPAVVFQTKRSKQVCADPSESWVQEYVYDLELN

SEQ ID NO: 12 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 13 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 14 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 15 - protein, 157 aa, Homo sapiens [sequence omitted]

SEQ ID NO: 16 - protein, 155 aa, Homo sapiens
YRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 17 - protein, 178 aa, Homo sapiens
QDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 18 - protein, 429 aa, Homo sapiens
SFTTRSTFSTNYRSLGSVQAPSYGARPVSSAASVYAGAGGSGSRISVSRSTSFRGGMGSGGLATGIAGGLAGMGGIQNEKETMQSLNDRLASYLDRVRSLETENRRLESKIREHLEKKGPQVRDWSHYFKIIEDLRAQIFANTVDNARIVLQIDNARLAADDFRVKYETELAMRQSVENDIHGLRKVIDDTNITRLQLETEIEALKEELLFMKKNHEEEVKGLQAQIASSGLTVEVDAPKSQDLAKIMADIRAQYDELARKNREELDKYWSQQIEESTTVVTTQSAEVGAAETTLTELRRTVQSLEIDLDSMRNLKASLENSLREVEARYALQMEQLNGILLHLESELAQTRAEGQRQAQEYEALLNIKVKLEAEIATYRRLLEDGEDFNLGDALDSSNSMQTIQKTTTRRIVDGKVVSETNDTKVLRH

SEQ ID NO: 19 - protein, 229 aa, Homo sapiens
APMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 20 - protein, 450 aa, Homo sapiens
FPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 21 - protein, 250 aa, Homo sapiens
LPLPQEAGGMSELQWEQAQDYLKRFYLYDSETKNANSLEAKLKEMQKFFGLPITGMLNSRVIEIMQKPRCGVPDVAEYSLFPNSPKWTSKVVTYRIVSYTRDLPHITVDRLVSKALNMWGKEIPLHFRKVVWGTADIMIGFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK